SEQUENCE LISTING (98 sequences)

SEQ ID NO: 1 - DNA, 19 bp, Artificial Sequence
  synthetic oligonucleotides
ctcgagcacctaggaggcg

SEQ ID NO: 2 - DNA, 20 bp, Artificial Sequence
  synthetic oligonucleotides
gctgttcctgtcgctgaacg

SEQ ID NO: 3 - DNA, 20 bp, Artificial Sequence
  synthetic oligonucleotides
cgtggtcaaacgcctaatgc

SEQ ID NO: 4 - DNA, 22 bp, Artificial Sequence
  synthetic oligonucleotides
cttgtcatcgttggattctggc

SEQ ID NO: 5 - DNA, 24 bp, Artificial Sequence
  synthetic oligonucleotides
cagcgatacaagaacaaacaccag

SEQ ID NO: 6 - DNA, 23 bp, Artificial Sequence
  synthetic oligonucleotides
gataataccgcgccacatagcag

SEQ ID NO: 7 - DNA, 22 bp, Artificial Sequence
  synthetic oligonucleotides
acagcggtaagatccttgagag

SEQ ID NO: 8 - DNA, 25 bp, Artificial Sequence
  synthetic oligonucleotides
cccgggatctgattaattaaaacgt

SEQ ID NO: 9 - DNA, 60 bp, Artificial Sequence
  synthetic oligonucleotides
gtaagccacgttttaattaatcagatcccgggctattcttttgatttataagggattttg

SEQ ID NO: 10 - DNA, 60 bp, Artificial Sequence
  synthetic oligonucleotides
ttagcaaacgtagaaaatacatacataaaggtggctgcgaataataattttttcacgttg

SEQ ID NO: 11 - DNA, 60 bp, Artificial Sequence
  synthetic oligonucleotides
gagattttcaacgtgaaaaaattattattcgcagccacctttatgtatgtattttctacg

SEQ ID NO: 12 - DNA, 60 bp, Artificial Sequence
  synthetic oligonucleotides
ggggtcgacgcggccgtggcgcgcctcctaggtgctcgagcccgagatagggttgagtgt

SEQ ID NO: 13 - DNA, 60 bp, Artificial Sequence
  synthetic oligonucleotides
aggagtcttaaaaaaaaaaaaccccgcgcggggtttttttttacgcgccctgtagcggcg

SEQ ID NO: 14 - DNA, 60 bp, Artificial Sequence
  synthetic oligonucleotides
aaatcggcaaaatcccttataaatcaaaagaatagcccgagatagggttgagtgttgttc

SEQ ID NO: 15 - DNA, 60 bp, Artificial Sequence
  synthetic oligonucleotides
caaactggaacaacactcaaccctatctcgggctattcttttgatttataagggattttg

SEQ ID NO: 16 - DNA, 60 bp, Artificial Sequence
  synthetic oligonucleotides
cttgcctcgagcacctaggaggcgcgccacgaaattgtaaacgttaatattttgttaaaa

SEQ ID NO: 17 - DNA, 60 bp, Artificial Sequence
  synthetic oligonucleotides
aatttaacgcgaattttaacaaaatattaacgtttacaatttcgtggcgcgcctcctagg

SEQ ID NO: 18 - DNA, 60 bp, Artificial Sequence
  synthetic oligonucleotides
tgagattatcaaaaaggatcttcacctagatccggaataggaacttcatttaaatggcgc

SEQ ID NO: 19 - DNA, 60 bp, Artificial Sequence
  synthetic oligonucleotides
gcgtaaggcgcgccatttaaatgaagttcctattccggatctaggtgaagatcctttttg

SEQ ID NO: 20 - DNA, 60 bp, Artificial Sequence
  synthetic oligonucleotides
atatacgtatactcttacttacttaagatcttttgaattcctcaaaggcggtaatacggt

SEQ ID NO: 21 - DNA, 60 bp, Artificial Sequence
  synthetic oligonucleotides
taccgcctttgaggaattcaaaagatcttaagtaagtaagagtatacgtatatcggctaa

SEQ ID NO: 22 - DNA, 60 bp, Artificial Sequence
  synthetic oligonucleotides
cctcagaagcgtagtccggaacgtcgtacgggtatgcgccatggtgatggtgatggtgac

SEQ ID NO: 23 - DNA, 60 bp, Artificial Sequence
  synthetic oligonucleotides
caccatggcgcatacccgtacgacgttccggactacgcttctgagggtggtggctctgag

SEQ ID NO: 24 - DNA, 60 bp, Artificial Sequence
  synthetic oligonucleotides
gcgtaaaaaaaaaccccgcgcggggttttttttttttaagactccttattacgcagtatg

SEQ ID NO: 25 - DNA, 20 bp, Artificial Sequence
  synthetic oligonucleotides
caccatcaccatcaccatgg

SEQ ID NO: 26 - DNA, 21 bp, Artificial Sequence
  synthetic oligonucleotides
tagaaagtctcctgtgcatga

SEQ ID NO: 27 - DNA, 60 bp, Artificial Sequence
  synthetic oligonucleotides
tgaaacatcttaatcatgcacaggagactttctagtgaaaaaattattattcgcaattcc

SEQ ID NO: 28 - DNA, 60 bp, Artificial Sequence
  synthetic oligonucleotides
cggaacgtcgtacgggtatgcgccatggtgatggtgatggtgagagccgccaccagaacc

SEQ ID NO: 29 - DNA, 27 bp, Artificial Sequence
  synthetic oligonucleotides
tattcttttgatttataagggattttg

SEQ ID NO: 30 - DNA, 20 bp, Artificial Sequence
  synthetic oligonucleotides
cccgagatagggttgagtgt

SEQ ID NO: 31 - DNA, 20 bp, Artificial Sequence
  synthetic oligonucleotides
ttcgccctttgacgttggag

SEQ ID NO: 32 - DNA, 22 bp, Artificial Sequence
  synthetic oligonucleotides
ccggttgataatcagaaaagcc

SEQ ID NO: 33 - DNA, 20 bp, Artificial Sequence
  synthetic oligonucleotides
cgctttcttcccttcctttc

SEQ ID NO: 34 - DNA, 20 bp, Artificial Sequence
  synthetic oligonucleotides
aagggcgaaaaaccgtctat

SEQ ID NO: 35 - DNA, 20 bp, Artificial Sequence
  synthetic oligonucleotide
tgattacggtgctgctatcg

SEQ ID NO: 36 - DNA, 20 bp, Artificial Sequence
  synthetic oligonucleotides
acaaaagggcgacattcaac

SEQ ID NO: 37 - DNA, 20 bp, Artificial Sequence
  synthetic oligonucleotides
ggcgattctgctaacaaagg

SEQ ID NO: 38 - DNA, 20 bp, Artificial Sequence
  synthetic oligonucleotides
ggcgagttccatagcttcag

SEQ ID NO: 39 - DNA, 60 bp, Artificial Sequence
  synthetic oligonucleotide
actgaaacatcttaatcatgcacaggagactttctaatgagcaaaggagaagaacttttc

SEQ ID NO: 40 - DNA, 60 bp, Artificial Sequence
  synthetic oligonucleotides
cgccttaatacgttattagccgatatacgtatactcttacttgcgcgcaattcccatgtc

SEQ ID NO: 41 - DNA, 60 bp, Artificial Sequence
  synthetic oligonucleotide
cttaacggctgacatgggaattgcgcgcaagtaagagtatacgtatatcggctaataacg

SEQ ID NO: 42 - DNA, 60 bp, Artificial Sequence
  synthetic oligonucleotides
gggacaactccagtgaaaagttcttctcctttgctcattagaaagtctcctgtgcatgat

SEQ ID NO: 43 - DNA, 18 bp, Artificial Sequence
  synthetic oligonucleotides
acgcgccctgtagcggcg

SEQ ID NO: 44 - DNA, 60 bp, Artificial Sequence
  synthetic oligonucleotides
cacacccgccgcgcttaatgcgccgctacagggcgcgtaaaggcggtaatacggttatcc

SEQ ID NO: 45 - DNA, 59 bp, Artificial Sequence
  synthetic oligonucleotides
gcttactgaatttcggtggtgccgttaattaaccggtgggtttttacgcgccctgtagc

SEQ ID NO: 46 - DNA, 60 bp, Artificial Sequence
  synthetic oligonucleotides
gagtgaccaagagaggatgaagcatttgccgagtagggaataggaacttcatttaaatgg

SEQ ID NO: 47 - DNA, 60 bp, Artificial Sequence
  synthetic oligonucleotides
ctactcggcaaatgcttcatcctctcttggtcactcggttggatctaggtgaagatcctt

SEQ ID NO: 48 - DNA, 60 bp, Artificial Sequence
  synthetic oligonucleotide
gtcggaaaagtggccatcatttgacgaactacagccgcgctatccacagaatcaggggat

SEQ ID NO: 49 - DNA, 60 bp, Artificial Sequence
  synthetic oligonucleotide
gcgcggctgtagttcgtcaaatgatggccacttttccgacgacatgggaattgcgcgcaa

SEQ ID NO: 50 - DNA, 60 bp, Artificial Sequence
  synthetic oligonucleotides
ggacattcaggagattttcgccggacgtacgcattttatttgtagagctcatccatgcca

SEQ ID NO: 51 - DNA, 60 bp, Artificial Sequence
  synthetic oligonucleotides
gcttactgaatttcggtggtgccgttaattaaccggtgggtaacggggtctgacgctcag

SEQ ID NO: 52 - DNA, 60 bp, Artificial Sequence
  synthetic oligonucleotides
aaccgagtgaccaagagaggatgaagcatttgccgagtagcgtcaggtggcacttttcgg

SEQ ID NO: 53 - DNA, 60 bp, Artificial Sequence
  synthetic oligonucleotides
ctactcggcaaatgcttcatcctctcttggtcactcggtttgtacggtacaaaccccgac

SEQ ID NO: 54 - DNA, 60 bp, Artificial Sequence
  synthetic oligonucleotides
taagcattttccctactgatgttcatcatctgagcgtattggttataagtgaacgatacc

SEQ ID NO: 55 - DNA, 60 bp, Artificial Sequence
  synthetic oligonucleotides
aagaacggacggtatcgttcacttataaccaatacgctcagatgatgaacatcagtaggg

SEQ ID NO: 56 - DNA, 60 bp, Artificial Sequence
  synthetic oligonucleotides
tcggaaaagtggccatcatttgacgaactacagccgcgcaacttcggaataggaacttcg

SEQ ID NO: 57 - DNA, 20 bp, Artificial Sequence
  synthetic oligonucleotides
aatgcgtacgtccggcgaaa

SEQ ID NO: 58 - DNA, 26 bp, Artificial Sequence
  synthetic oligonucleotides
tagaaagtctcctgtgcatgattaag

SEQ ID NO: 59 - DNA, 60 bp, Artificial Sequence
  synthetic oligonucleotides
actgaaacatcttaatcatgcacaggagactttctaatgagtgttttagtgtattctttc

SEQ ID NO: 60 - DNA, 60 bp, Artificial Sequence
  synthetic oligonucleotides
tttcgggacattcaggagattttcgccggacgtacgcatttcatgaggaagtttccatta

SEQ ID NO: 61 - DNA, 58 bp, Artificial Sequence
  synthetic oligonucleotides
ggcgcatacccgtacgacgttccggactacgcttctgagggtggtggctctgagggtg

SEQ ID NO: 62 - DNA, 60 bp, Artificial Sequence
  synthetic oligonucleotides
ttcgggacattcaggagattttcgccggacgtacgcattttaagactccttattacgcag

SEQ ID NO: 63 - DNA, 60 bp, Artificial Sequence
  synthetic oligonucleotides
cgtactgaaacatcttaatcatgcacaggagactttctagtgaagtatttacttcccacg

SEQ ID NO: 64 - DNA, 19 bp, Artificial Sequence
  synthetic oligonucleotides
cctcagaagcgtagtccgg

SEQ ID NO: 65 - DNA, 60 bp, Artificial Sequence
  synthetic oligonucleotides
ctaaaacgaaagaggcgaaagaatacacttaaacactcacctttgacccccagcgattat

SEQ ID NO: 66 - DNA, 60 bp, Artificial Sequence
  synthetic oligonucleotides
gcgcttggtataatcgctgggggtcaaaggtgagtgtttaagtgtattctttcgcctctt

SEQ ID NO: 67 - DNA, 60 bp, Artificial Sequence
  synthetic oligonucleotides
agactttttaagtttttcaaagttgatgatgctcattatgaggaagtttccattaaacgg

SEQ ID NO: 68 - DNA, 60 bp, Artificial Sequence
  synthetic oligonucleotides
ttcctcataatgagcatcatcaactttgaaaaacttaaaaagtctttagtcctcaaagcc

SEQ ID NO: 69 - DNA, 49 bp, Artificial Sequence
  synthetic oligonucleotides
gattacaaagatgatgatgataagatgagtgttttagtgtattctttcg

SEQ ID NO: 70 - DNA, 29 bp, Artificial Sequence
  synthetic oligonucleotides
cattagaaagtctcctgtgcatgattaag

SEQ ID NO: 71 - DNA, 60 bp, Artificial Sequence
  synthetic oligonucleotides
tcgtactgaaacatcttaatcatgcacaggagactttctaatgaagtatttgctgccaac

SEQ ID NO: 72 - DNA, 60 bp, Artificial Sequence
  synthetic oligonucleotides
atacactaaaacactcatcttatcatcatcatctttgtaatcaccacccgtttccggcaa

SEQ ID NO: 73 - DNA, 60 bp, Artificial Sequence
  synthetic oligonucleotides
aattgtgagcggataacaatttcacacaggaaacagctattaccgcgagacccacgctca

SEQ ID NO: 74 - DNA, 60 bp, Artificial Sequence
  synthetic oligonucleotides
aattcgcattaaatttttgttaaatcagctcattttttaaggaaagccacgttgtgtctc

SEQ ID NO: 75 - DNA, 60 bp, Artificial Sequence
  synthetic oligonucleotides
gagattttgagacacaacgtggctttccttaaaaaatgagctgatttaacaaaaatttaa

SEQ ID NO: 76 - DNA, 60 bp, Artificial Sequence
  synthetic oligonucleotides
gctgataaatctggagccggtgagcgtgggtctcgcggtaatagctgtttcctgtgtgaa

SEQ ID NO: 77 - DNA, 60 bp, Artificial Sequence
  synthetic oligonucleotides
aggctccttttggagccttttttttggagattttcaacgtgaagtatttacttcccacgg

SEQ ID NO: 78 - DNA, 20 bp, Artificial Sequence
  synthetic oligonucleotides
ctccggcctttctcaccctt

SEQ ID NO: 79 - DNA, 60 bp, Artificial Sequence
  synthetic oligonucleotides
ataagccggctgcggccgtgggaagtaaatacttcacgttgaaaatctccaaaaaaaagg

SEQ ID NO: 80 - DNA, 21 bp, Artificial Sequence
  synthetic oligonucleotides
gattctaacgaggaaagcacg

SEQ ID NO: 81 - DNA, 25 bp, Artificial Sequence
  synthetic oligonucleotides
ccctcatatattttaaatgcaatgc

SEQ ID NO: 82 - DNA, 21 bp, Artificial Sequence
  synthetic oligonucleotides
cgtactatggttgctttgacg

SEQ ID NO: 83 - DNA, 60 bp, Artificial Sequence
  synthetic oligonucleotides
caccaaaccgcccccggtctcgactaattgtacctgggccatagagccgccaccagaacc

SEQ ID NO: 84 - DNA, 60 bp, Artificial Sequence
  synthetic oligonucleotides
gtatatattaatgtatcgattaaataaggaggaataaaccatggcccaggtacaattagt

SEQ ID NO: 85 - DNA, 58 bp, Artificial Sequence
  synthetic oligonucleotides
ccatatggtaccagctgcagatctcgagctcggatccttaagcgtagtccggaacgtc

SEQ ID NO: 86 - DNA, 20 bp, Artificial Sequence
  synthetic oligonucleotides
tgttctttcctgcgttatcc

SEQ ID NO: 87 - DNA, 60 bp, Artificial Sequence
  synthetic oligonucleotides
caaaccgcccccggtctcgactaattgtacctgggccatggtttattcctccttatttaa

SEQ ID NO: 88 - DNA, 60 bp, Artificial Sequence
  synthetic oligonucleotides
caccatggcgcatacccgtacgacgttccggactacgcttaaggatccgagctcgagatc

SEQ ID NO: 89 - DNA, 22 bp, Artificial Sequence
  synthetic oligonucleotides
ctcaaaggcggtaatacggtta

SEQ ID NO: 90 - DNA, 60 bp, Artificial Sequence
  synthetic oligonucleotides
gtactgaaacatcttaatcatgcacaggagactttctagtgaaatacttgttgcccacag

SEQ ID NO: 91 - DNA, 60 bp, Artificial Sequence
  synthetic oligonucleotides
acactaaaacactcatcttatcatcatcatctttgtaatcaccgtcgatacattgatcga

SEQ ID NO: 92 - DNA, 34 bp, Artificial Sequence
  synthetic oligonucleotides
gaattcaaaagatcttaagtaagtaagagtatac

SEQ ID NO: 93 - DNA, 20 bp, Artificial Sequence
  synthetic oligonucleotides
cccaccggttaattaacggc

SEQ ID NO: 94 - DNA, 453 bp, Artificial Sequence
  synthetic oligonucleotides
atgaagtatttgctgccaaccgccgctgctgggctcctgcttctggcggctcagcctgcgatggcccaggttcagttggtcgaatctggtggtggtttggttgaaccaggcgggagcctgcgcttaagttgcgcggcttctgggataatatttaaaataaatgacatgggatggtatcggcaggcaccgggtaagcgccgcgagtgggtggccgcctctactggcggggatgaggcaatctatcgcgactctgttaaggaccgctttaccattagcagagacgcaaaaaacagcgtgtttctgcagatgaatagcttaaagcccgaggacacggctgtttactattgtactgcggtcataagcacggatagagatggtacggaatggaggcgatattggggccaaggcacccaagtaacggtctcgtcagggggtttgccggaaacgggtggt

SEQ ID NO: 95 - DNA, 429 bp, Artificial Sequence
  synthetic oligonucleotides
atgaaatatctgttaccgacagcagctgctggtttattattgttagctgcccagcctgcaatggcacaggttcagttggtagaatcgggcgggggtttagcccagccggggggttcacttcgcctttcctgtgctgcttcgggatcgacaatctctagtgttgctgttggttggtaccgtcagacacctgggaatcaacgtgaatgggtggctaccagctcaacttcctctaccactgccacgtatgcagattcggtgaagggccgttttacaattagtcgtgacaacgccaaaaatacaatttatctgcagatgaatagtctgaagcccgaagatacagccgtctactattgcaagacaggcttgactaactggggtcgcggaactcaagttacagtgtctagcggcggcttgcctgagactggaggt

SEQ ID NO: 96 - DNA, 432 bp, Artificial Sequence
  synthetic oligonucleotides
atgaagtatttacttcccacggccgcagccggcttattacttcttgctgctcagccagcaatggcccaggtacaattagtcgagaccgggggcggtttggtgcagccaggcggtagtctgcgcctgtcatgtacagcaagtggcttcacgttctctatgcacgctatgacatggtaccgccaggctcctggaaaacaacgtgaattggttgctgttatcacgagtcatggggatcgtgcgaattatacagactcggtccgtgggcgctttactatctcccgcgacaacacaaagaacatggtgtatttgcagatgaactccctgaaacccgaggatactgccgtctactactgtaacgtcccacgttatgatagttggggtcaaggtacgcaggtgaccgtaagctcagggggtctgccggagacaggaggt

SEQ ID NO: 97 - DNA, 600 bp, Artificial Sequence
  synthetic oligonucleotides
atgaaatacttgttgcccacagcggcggccggcttgctgttattggcggcgcaacctgcgatggcccaggattctactagcgatcttattccggcaccgcccttatcgaaagtcccattacaacaaaacttccaggacaatcaattccacggtaagtggtacgttgtcggcttggctgggaatcgtattttgcgcgacgaccagcacccaatgaatatgtacgcgacgatttatgagctgaaggaagacaaatcctataatgtgacttccgtgatcagtagtcacaaaaaatgtgagtacaccatcgctaccttcgtaccaggctctcaaccaggggagttcaccttagggaacatcaagtcctatggagataaaacctcctatttagtacgcgtcgtctccaccgattacaatcagtatgctatggttttctttaaacttgctgaagataacgcggagttctttgctatcacaatctacgggcgcactaaggagctggcgtccgagttgaaggaaaacttcatccgcttctccaagagcctggggcttcccgaaaaccacatcgtatttcctgtaccgatcgatcaatgtatcgacggt

SEQ ID NO: 98 - DNA, 1344 bp, Artificial Sequence
  synthetic oligonucleotides
atgaagtatttacttcccacggccgcagccggcttattacttcttgctgctcagccagcaatggcctcgaataacgctttacaaacaattattaacgcccggttaccaggcgaagaggggctgtggcagattcatctgcaggacggaaaaatcagcgccattgatgcgcaatccggcgtgatgcccataactgaaaacagcctggatgccgaacaaggtttagttataccgccgtttgtggagccacatattcacctggacaccacgcaaaccgccggacaaccgaactggaatcagtccggcacgctgtttgaaggcattgaacgctgggccgagcgcaaagcgttattaacccatgacgatgtgaaacaacgcgcatggcaaacgctgaaatggcagattgccaacggcattcagcatgtgcgtacccatgtcgatgtttcggatgcaacgctaactgcgctgaaagcaatgctggaagtgaagcaggaagtcgcgccgtggattgatctgcaaatcgtcgccttccctcaggaagggattttgtcgtatcccaacggtgaagcgttgctggaagaggcgttacgcttaggggcagatgtagtgggggcgattccgcattttgaatttacccgtgaatacggcgtggagtcgctgcataaaaccttcgccctggcgcaaaaatacgaccgtctcatcgacgttcactgtgatgagatcgatgacgagcagtcgcgctttgtcgaaaccgttgctgccctggcgcaccatgaaggcatgggcgcgcgagtcaccgccagccacaccacggcaatgcactcctataacggggcgtatacctcacgcctgttccgcttgctgaaaatgtccggtattaactttgtcgccaacccgctggtcaatattcatctgcaaggacgtttcgatacgtatccaaaacgtcgcggcatcacgcgcgttaaagagatgctggagtccggcattaacgtctgctttggtcacgatgctgtcttcgatccgtggtatccgctgggaacggcgaatatgctgcaagtgctgcatatggggctgcatgtttgccagttgatgggctacgggcagattaacgatggcctgaatttaatcacccaccacagcgcaaggacgttgaatttgcaggattacggcattgccgccggaaacagcgccaacctgattatcctgccggctgaaaatgggtttgatgcgctgcgccgtcaggttccggtacgttattcggtacgtggcggcaaggtgattgccagcacacaaccggcacaaaccaccgtatatctggagcagccagaagccatcgattacaaacgt

(19) United States
(12) Patent Application Publication
(10) Pub. No.: US 2023/0372419 A1
(43) Pub. Date: Nov. 23, 2023

(54) LIVE BIOTHERAPEUTICS SECRETING SYNTHETIC BACTERIOPHAGES IN THE TREATMENT OF CANCER

(71) Applicant: TATUM BIOSCIENCE INC., Sherbrooke (CA)

(72) Inventors: Samuel GENIER, Sherbrooke (CA); Emilie ST-PIERRE, Sherbrooke (CA); Kevin NEIL, Sherbrooke (CA); Sebastien RODRIGUE, Sherbrooke (CA); Jean-Francois MILLAU, Sherbrooke (CA)

(21) Appl. No.: 18/030,407

(22) PCT Filed: Oct. 7, 2021
(86) PCT No.: PCT/CA2021/051415

§ 371 Date: Apr. 5, 2023

Related U.S. Application Data
(60) Provisional application No. 63/088,643, filed on Oct. 7, 2020.
(60) Provisional application No. 63/161,543, filed on Mar. 16, 2021.
(60) Provisional application No. 63/215,176, filed on Jun. 25, 2021.

(51) Int. Cl. A61K 35/76; C12N 9/78; C12N 15/86; C07K 16/28; A61P 35/00

(52) U.S. Cl. CPC A61K 35/76; C12N 9/78; C12N 15/86; C07K 16/2827; C07K 16/2818; A61P 35/00; C12Y 305/04001

(57) ABSTRACT

The present disclosure generally relates to a synthetic therapeutic bacteriophage displaying at least one therapeutic agent, wherein the at least one therapeutic agent is fused to a coating protein of the synthetic bacteriophage.

### CROSS-REFERENCE TO RELATED APPLICATIONS

This application claims the benefit of and priority to U.S. provisional patent application No. 63/088,643, filed on Oct. 7, 2020, on U.S. provisional patent application No. 63/161,543, filed on Mar. 16, 2021, and on U.S. provisional patent application No. 63/215,176, filed on Jun. 25, 2021, the content of these applications is herein incorporated in its entirety by reference.

### FIELD OF TECHNOLOGY

The present disclosure generally relates to a synthetic bacteriophage displaying one or more recombinant molecules with anti-tumoral activities, to live biotherapeutic expressing and delivering such synthetic bacteriphage, and to methods of preventing and treating cancer using same.

### BACKGROUND INFORMATION

Despite important advancement in cancer research and treatments, cancer remains the second most prevalent cause of death in industrialized countries (Siegel R et al. ACS Journal, Cancer statistics 2021; incorporated herein by reference). Cancer is a complex and difficult disease to treat, often requiring to act on several therapeutic targets simultaneously to maximize chances of treatment success. This strategy is called combination therapy, clinicians treat patients by combining two or more therapeutic agents (Mokhtari et al. Oncotarget 2017 Jun. 6; 8(23):38022-38043; incorporated herein by reference). By targeting different pathways to inhibit or eliminate cancerous cells, combination therapy provides better results than mono therapy and has become a cornerstone of cancer treatment.

The necessity to combine therapies in order to maximize treatment outcomes is exemplified in the field of immuno-oncology, which is a branch of cancer therapy that manipulates the immune system to trigger tumor clearance. In immuno-oncology, tumor clearance is greatly improved when a tumor is considered “hot” (Duan et al., Trends in Cancer (2020), Volume 6, Issue 7, p605-618; incorporated herein by reference). This happens when two conditions are fulfilled: (i) immune cells are present within the tumor and (ii) these immune cells are not repressed by the tumor microenvironment. A current strategy to turn cold tumors into hot tumors is to use two drugs, a first one to promote the recruitment of immune cells and tumor infiltration, and a second one to ensure that the immune cells are active and not inhibited by the tumor microenvironment (Haanen J. et al., Cell (2017), Volume 170, Issue 6, p 1055-1056 and Sevenich L., Front. Oncol. (2019), Volume 9, Article 163; incorporated herein by reference). For instance, this is done by combining oncolytic virus treatment (e.g. AMGEN Talimogene Laherparepvec), which promotes tumor infiltration, with checkpoint inhibitors (e.g. Bristol-Myers Squibb ipilimumab), which ensures that the immune cells are activated (Puzanov I. et al., J Clin Oncol (2016), 1; 34(22):2619-26; incorporated herein by reference).

While combining several treatment modalities provides clear therapeutic benefits compared to monotherapies, such strategy possesses at least two major drawbacks. First, combining several treatments also combines their side effects. For instance, combining a PD-L1 checkpoint inhibitor with at CTLA-4 checkpoint inhibitor can provide better results than mono-therapy but also results in adverse events in about 50% of patients (Grover S. et al., Gastrointestinal and Hepatic Toxicities of Checkpoint Inhibitors: Algorithms for Management 2018 ASCO Educational book; incorporated herein by reference). This can have severe consequences, as excessive side effects sometimes prompt to prematurely end the treatments, leaving patients with no therapeutic solutions. Secondly, combining several treatments also results in combining the costs of development for each one of these treatments, which in turn inflates the cost of treatment.

Parallel to that, anticancer therapies typically rely on toxic mechanisms to eliminate cancer cells. Because most anticancer drugs are administrated systematically, and diffuse through the entire body, they exert their toxic effect on healthy tissues and organs, which in turns produces side effects (Cleeland, C. S. et al. Nat. Rev. Clin. Oncol. (2012), 9, 471-478; incorporated herein by reference).

Most current cancer treatments are thus suffering from a lack of targeted delivery approach. Those treatments instead rely on high doses to reach the desired intratumoral concentration for optimal therapeutic activity at tumor sites, which increases risks of side effects.

In view of the above, there remains a need in the field of cancer treatment for a therapeutic agent capable of overcoming at least some of the drawbacks identified above. In particular for a therapeutic agent capable of acting on several therapeutic targets simultaneously, while limiting side effects by localized delivery and high efficacy at low dose.

### BRIEF SUMMARY

According to various aspects, the present technology relates to a synthetic therapeutic bacteriophage displaying at least one therapeutic agent, wherein the at least one therapeutic agent is fused to a coating protein of the synthetic bacteriophage. In some implementations of these aspects, the synthetic bacteriophage secretion system comprises a synthetic bacteriophage machinery. The synthetic bacteriophage machinery comprises a bacteriophage assembly module, a bacteriophage replication module, a bacteriophage coating module, and a therapeutic module. In some instances, the bacteriophage assembly module comprises: i) bacteriophage gene gpI, encoding the proteins pI and pXI; or ii) bacteriophage gene gpIV, encoding for the protein pIV; or iii) both i) and ii). In some other instances, the bacteriophage replication module comprises: i) bacteriophage gene gpII, encoding proteins pII and pX; or ii) bacteriophage gene gpV, encoding protein pV; or iii) both i) and ii). In some instances, the bacteriophage coating module comprises: bacteriophage genes gpIII, gpVI, gpVII, gpVIII, and gpIX, or a portion thereof, respectively coding for coating protein pill, pVI, pVII, pVIII, and pIX or coding for a portion thereof. In some instances, the therapeutic module comprises one or more bacteriophage coating genes selected from gpIII, gpVI, gpVII, gpVIII, and gpIX, respectively coding for coating protein pill, pVI, pVII, pVIII, and pIX. In some implementations, the at least one therapeutic agent is displayed on the at least some of the coating proteins.

According to various aspects, the therapeutic agent is a binding protein. In some instances, the binding protein binds to and inhibits one or more proteins, peptides, or molecule involved in carcinogenesis, development of cancer, or of metastases. In some other instances, the one or more proteins, peptides, or molecule to be inhibited are selected from: CSF1, CSF1R, CCR4, CCL2, CCL17, CCL22, HER2, GD2, IL-1β, IL-6, IL-10, IL-13, IL-17, IL-27, IL-35, CD20, CD27, CD30, CD33, CD70, TGF-β, M-CSF, EGFR, ERBB2, ERBB3, PGE2, VEGF, VEGFR-2, CXCR4/CXCL12, Tie2, galectin-1, galectin-3, Phosphatidyl serine, and TAM and Tim Phosphatidyl serine receptors. In some instances, the binding protein acts as agonists to activate co-stimulatory receptor that lead to the elimination of cancerous cells, wherein the one or more co-stimulatory cellular receptors are selected from, but not limited to CD40, CD27, CD28, CD70, ICOS, CD357, CD226, CD137, and CD134. In some other instances, the binding protein inhibits an immune checkpoint molecule such as, but not limited to: CCR4, CTLA-4, CD80, CD86, PD-1, PD-L1, PD-L2, TIGIT, VISTA, LAG-3, TIM1, TIM3, CEACAM1, LAIR-1, HVEM, BTLA, CD47, SIRPα, CD160, CD200, CD200R, CD39, CD73, B7-H3, B7-H4, IDO, TDO, KIR, and A2aR.

According to various aspects, the therapeutic agent stimulates an immune response.

According to various aspects, the therapeutic agent is an antibody or antibody mimetics or a nanobody.

According to various aspects, the therapeutic agent is a cytosine deaminase.

According to various aspects, the present technology relates to a live biotherapeutic for producing and/or delivering at least one therapeutic agent, the live biotherapeutic comprising a recombinant bacterial organism comprising a synthetic bacteriophage secretion system capable of secreting the synthetic therapeutic bacteriophage as defined herein.

According to various aspects, the present technology relates to a live biotherapeutic for producing and/or delivering a therapeutic agent, the live biotherapeutic comprising a recombinant bacterial organism comprising a synthetic bacteriophage secretion system capable of secreting a synthetic therapeutic bacteriophage, wherein the synthetic therapeutic bacteriophage displays the therapeutic agent. In some aspects, the recombinant bacterial organism is selected from the Enterobacteriaceae family, the Pseudomonadaceae family and the Vibrionaceae family. In some aspects, the recombinant bacterial organism is a tumor targeting bacteria such as but not limited to: Escherichia coli Nissle 1917 and Escherichia coli MG1655.

According to various aspects, the present technology relates to a method for delivering at least one therapeutic agent to a tumor site in a subject, the method comprising administering an effective amount of the synthetic therapeutic bacteriophage as defined herein or an effective amount of the live biotherapeutic as defined herein to the subject in need thereof.

According to various aspects, the present technology relates to a method for prevention and/or treatment of cancer in a subject in need thereof, the method comprising administering an effective amount of the synthetic therapeutic bacteriophage as defined herein or an effective amount of the live biotherapeutic as defined herein to the subject in need thereof.

According to various aspects, the present technology relates to a method for prevention and/or treatment of cancer in a subject in need thereof, the method comprising administering an effective amount of a synthetic therapeutic bacteriophage to the subject in need thereof, wherein the synthetic bacteriophase does not display a therapeutic agent. In some implementations, the cancer is selected from adrenal cancer, adrenocortical carcinoma, anal cancer, appendix cancer, bile duct cancer, bladder cancer, bone cancer, brain cancer, bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, largyngeal cancer, hypopharyngeal cancer, leukemia, liver cancer, lung cancer, lymphoma, malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer, small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenstrom macrogloblulinemia, and Wilms tumor.

According to various aspects, the present technology relates to the use of an effective amount of the synthetic therapeutic bacteriophage as defined herein or of an effective amount of the live biotherapeutic as defined herein for prevention and/or treatment of a cancer in a subject in need thereof.

According to various aspects, the present technology relates to the use of an effective amount of a synthetic therapeutic bacteriophage for prevention and/or treatment of a cancer in a subject in need thereof, wherein the synthetic therapeutic bacteriophase does not display a therapeutic agent.

According to various aspects, the present technology relates to the use of a kit comprising the synthetic therapeutic bacteriophage of any one of claims 1 to 33 or the live biotherapeutic as defined together with instructions for administration of the synthetic therapeutic bacteriophage or of the live biotherapeutic to a subject.

According to various aspects, the present technology relates to a kit comprising the live biotherapeutic as defined herein together with instructions for administration of the drug to a subject.

### BRIEF DESCRIPTION OF THE DRAWINGS

FIG. 1 is a schematic representation of an examplified configuration of the live biotherapeutic secreting a synthetic therapeutic bacteriophage according to one embodiment of the present technology.

FIG. 2 is a schematic representation of mono-, bi-, and multi-therapeutic synthetic bacteriophages displaying mono- and/or bi-specific therapeutic proteins.

FIG. 3 is a schematic representation of a live biotherapeutic secreting CD47 binding synthetic therapeutic bacteriophages mode of action. The live biotherapeutic secrete a synthetic bacteriophage displaying a checkpoint inhibitor, the anti-CD47 nanobody. The CD47 nanobody recognizes and binds to the CD47 immune checkpoint expressed on cancer cells. The therapeutic bacteriophage thus binds to CD47 and prevents the CD47 immune checkpoint from inhibiting T-cells activation.

FIG. 4 is a schematic representation of the immunogenic effect of the synthetic bacteriophage and the bacterial host.

FIG. 5 is a schematic representation of example conformation of the synthetic bacteriophage secretion system. Examples of built synthetic bacteriophage machinery conformation comprise M13K07 (A), M13mp18-Kan (B), pTAT004 (C), pTAT025 (D) and their derivatives. Examples of built synthetic bacteriophage scaffold vector include pTAT002 (E), pTAT012 (F), pTAT013 (G), pTAT014 (H) and their derivative.

FIG. 6 comprises a schematic representation of a map of pTAT001 is shown and sites cleaved by restriction enzymes selected to validate the construction are identified on the map. Expected digestion products are also shown on the map as well as the experimental agarose gel of the construction after digestion with the specified enzymes.

FIGS. 7A-7C are graphs showing that the live biotherapeutic secretes fully assembled bacteriophages displaying the checkpoint inhibitor fused to pill. (A) The infectivity of bacteriophage secreted by the live biotherapeutic comprising pTAT004 with either pTAT002 (control), or pTAT003 (displaying anti-CD47 nanobody) compared to the infectivity of M13K07 comprise. (B) Dosage by ELISA of the synthetic bacteriophage produced by the live biotherapeutic displaying either nothing (pTAT004+pTAT002), nanobodies on pIII (anti-CD47, pTAT004+pTAT003; anti-PD-L1, pTAT004+pTAT020; anti-CTLA-4, pTAT004+pTAT019), an anticalin on pIII (anti-CTLA-4, pTAT004+pTAT030), an enzyme on pIII (cytosine deaminase, pTAT004+pTAT022), a peptid on pVIII (pTAT002+pTAT027), or an anti-CD47 nanobody on pIX (pTAT025+pTAT002+pTAT028). Detection performed with an anti-pVIII B62-FE3 (progen) antibody coupled to HRP. (C) ELISA dosage of the bacteriophage produced by the live biotherapeutic bearing either pTAT004+pTAT002, pTAT004+pTAT003 or M13K07. Detection was performed with an anti-HA antibody coupled with HRP.

FIG. 8 is a graph showing that the synthetic bacteriophage strongly binds A20 lymphoma cancerous cells. A pull-down assay was performed using bacteriophage produced by the live biotherapeutic bearing pTAT004 and either pTAT002 (control) or pTAT003 (displaying the anti-CD47 nanobody).

FIGS. 9A-9L are graphs showing the synthetic bacteriophages displaying a checkpoint inhibitor hide immune checkpoints on cancer cells. (A) Fluorescence basal signal measured on unstained A20 cells using the FITC channel during a flow cytometry assay. (B) Fluorescence signal of an A20 population stained with the anti-CD47-FITC antibody. (C) Fluorescence signal of an A20 population first incubated with a control synthetic bacteriophages, produced by a live biotherapeutic bearing pTAT004+pTAT002, and then stained with the anti-CD47-FITC antibody. (D) Fluorescence signal of an A20 population first incubated with synthetic bacteriophages which displays an anti-CD47 nanobody on pIII, produced by a live biotherapeutic bearing pTAT004+pTAT003, and then stained with the anti-CD47-FITC antibody. (E) Fluorescence basal signal measured on unstained A20 cells using the FITC channel during of flow cytometry assay. (F) Fluorescence signal of an A20 population stained with the anti-CD47-FITC antibody. (G) Fluorescence signal of an A20 population first incubated with a control synthetic bacteriophage, produced by a live biotherapeutic bearing pTAT004+pTAT002, and then stained with the anti-CD47-FITC antibody. (H) Fluorescence signal of an A20 population first incubated with a synthetic bacteriophage displaying the anti-CD47 nanobody on pIX, produced by a live biotherapeutic bearing pTAT002+pTAT025+pTAT028, and then stained with the anti-CD47-FITC antibody. (I) Fluorescence basal signal measured on unstained A20 cells using the PE channel during flow cytometry assay. (J) Fluorescence signal of an A20 population stained with the anti-PD-L1-PE antibody. (K) Fluorescence signal of an A20 population first incubated with control synthetic bacteriophages, produced by a live biotherapeutic bearing pTAT004+pTAT002, and then stained with the anti-PD-L1-PE antibody. (L) Fluorescence signal of an A20 population first incubated with synthetic bacteriophages displaying an anti-PD-L1 nanobody on pIII, produced by a live biotherapeutic bearing pTAT004+pTAT020, and then stained with the anti-PD-L1-PE antibody.

FIG. 10 is a graph showing that the synthetic bacteriophage displaying an anti-CTLA-4 nanobody, or anticalin, can bind to CTLA-4 protein. An ELISA was conducted were synthetic bacteriophage displaying an anti-CTLA-4 nanobody (pTAT019), or an anti-CTLA-4 anticalin (pTAT030), or not (pTAT002).

FIGS. 11A-11E are graphs showing functional therapeutic protein remaining functional when cloned between two protein domains. The synthetic bacteriophage displaying an anti-PD-L1 nanobody inserted in pIII can bind to the PD-L1 protein on the surface of A20 cells and compete with PE labeled antibody. (A) Fluorescence basal signal measured on unstained A20 cells using the PE channel during of flow cytometry assay. (B) Fluorescence signal of an A20 population stained with the anti-PD-L1-PE antibody. (C) Fluorescence signal of an A20 population first incubated with a control synthetic bacteriophage (pTAT002, no display) and then stained with the anti-PD-L1-PE antibody. (D) Fluorescence signal of an A20 population first incubated with a synthetic bacteriophage displaying the anti-PD-L1 nanobody at the N-terminal end of pIII (pTAT032) and then stained with the anti-PD-L1-PE antibody. (E) Fluorescence signal of an A20 population first incubated with a synthetic bacteriophage displaying the anti-PD-L1 nanobody inserted between the binding domain and the bacteriophage anchor domain of pIII (pTAT033) and then stained with the anti-PD-L1-PE antibody.

FIGS. 12A-12F are graphs showing that the synthetic bacteriophage can display antigens on all its major coat protein pVIII subunits. (A) Sanger sequencing of the pTAT027 construction. (B) Bacteriophage particles secreted by the live biotherapeutic presenting or not the OVA epitope on pVIII and displaying or not the anti-CD47 nanobody on pIII measured by ELISA. (C) Western blot analysis of the protein profile of the pIII subunit in bacteriophages displaying or not OVA on pVIII. (D-F) Flow cytometry analysis of bacteriophages binding to CD47 on the surface of A20 cells. (D), incubated with pVIII-OVA+pIII wildtype bacteriophages and stained with anti-CD47-FITC antibody (E), or incubated with pVIII-OVA+nbCD47-pIII bacteriophages and stained with anti-CD47-FITC antibody (F). Reduction of the staining intensity is correlated with the masking of the CD47 on the surface of A20 cells.

FIGS. 13A-13B are graphs showing that displaying anti-CD47, anti-PD-L1, or anti-CTLA-4 nanobodies potentiate the antitumoral activity of synthetic bacteriophages. (A) Average tumor volume was measured for each mice groups treated with three intra-tumoral injections of bacteriophage particles ranging from 10⁷ to 10¹¹ control synthetic bacteriophage (pTAT002). For PBS, 10⁷, 10⁸, and 10⁹ bacteriophage particles treatments, doses were administered on days 0, 4, and 7 (arrows); while for the 10¹¹ bacteriophage particles treatment, doses were administered on days 0, 4, and 11 (gray arrow). (B) Tumor clearance observed with mice treated with control synthetic bacteriophage treatments. (C) Tumor volume measured in mice treated with three intra-tumoral injections (arrows) of 1×10⁸ control synthetic bacteriophages without therapeutic proteins (pTAT002), 1×10⁸ synthetic bacteriophages displaying the anti-PDL1 nanobody or 1×10⁸ synthetic bacteriophages displaying the anti-CTLA-4 nanobody. Individual tumor volume is shown for each mice (solid lines=cleared mice, doted lines=non-cleared mice). (A-B) Data is representative of at least 5 mice per group. Tumor volume was calculated by multiplying the largest measure by the square of the perpendicular measure divided by two.

FIGS. 14A-14H demonstrate the synergistic effect of a checkpoint inhibitor displayed by a synthetic bacteriophage. (A) ELISA assay demonstrating that purified anti-PD-L1 nanobody is functional and binds to the PD-L1 protein. (B-C) Tumors were engrafted in mice by injecting 5×106 A20 cells in their right flank. Tumor when then treated when tumor volumes ranged between 100-200 mm³. Individual tumor volume was measured for each mice treated on day 0, 4, and 7 with intra-tumoral injection of either PBS, 8×10¹⁵ molecules of anti-PD-L1 nanobody, 1×10⁸ of control synthetic bacteriophage particles (pTAT002), 5×10⁸ molecules of anti-PD-L1 nanobody, 1×10⁸ of control synthetic bacteriophage particle in conjunction with 5×10⁸ molecules of anti-PD-L1 nanobody, or 1×10⁸ synthetic bacteriophage particles displaying the anti-PD-L1 nanobody. Tumor clearance data are reported in (B), while tumor volume was calculated by multiplying the largest measure by the square of the perpendicular measure divided by two (solid lines=cleared mice, doted lines=non-cleared mice) (C).

FIG. 15 is a graph showing a live biotherapeutic secreting synthetic bacteriophage displaying an anti-CD47 nanobody inhibits tumor growth. Tumors were engrafted in mice by injecting 5×10⁶ A20 cells in their right flank. Tumor when then treated when tumor volumes ranged between 100-200 mm³, by injecting 100 μL of PBS (vehicle control), 5×10⁸ of live biotherapeutics secreting synthetic bacteriophages that do not display any therapeutic protein (pTAT002), or 5×10⁸ of live biotherapeutics secreting synthetic bacteriophages displaying an anti-CD47 nanobody the pIII sub-units (pTAT003). Tumor volume was measured at specified timepoints using digital calipers. Tumor volume was calculated by multiplying the largest measure by the square of the perpendicular measure divided by two. Only the treatment with synthetic bacteriophages displaying the CD47 checkpoint inhibitor induced tumor elimination.

FIGS. 16A-16B are graphs showing that a live biotherapeutic secreting synthetic bacteriophage displaying an anti-PD-L1 nanobody produces an anti-tumoral response. Tumors were engrafted in mice by injecting 5×10⁶ A20 cells in their right flank. Tumor were then treated, when tumor volumes ranged between 80-250 mm³, by injecting 50 μL of PBS (vehicle control), 5×10⁸ of live biotherapeutics secreting the control synthetic bacteriophages that do not display any therapeutic protein (pTAT002), or 5×10⁸ of live biotherapeutics secreting synthetic bacteriophages displaying an anti-PD-L1 nanobody on the pIII sub-units (pTAT020). (A) Tumor volume was measured at specified timepoints using digital calipers. Tumor volume was calculated by multiplying the largest measure by the square of the perpendicular measure divided by two (solid lines=cleared mice, doted lines=non-cleared mice). (B) Total clearance of the tumors from mice was evaluated at day 24 post-treatment for all mice groups. Mice were sacrificed and dissected to search for metastases and evaluate total clearance of the primary tumors. Mice with no more primary tumor and no detectable metastase at day 24 are considered to be cleared from cancer cells.

FIGS. 17A-17C are graphs showing that both the live biotherapeutic and the synthetic therapeutic bacteriophage elicit a long lasting adaptive immune response against cancerous cells. Mice bearing A20 tumors on their right flanks were treated by intratumoral injections at day 0, 3, and 11 with either synthetic bacteriophages displaying an anti-CD47 nanobody (A), or the live biotherapeutic secreting synthetic bacteriophages displaying an anti-CD47 nanobody (B). Once cleared from their tumors, mice were kept for 45 days post-treatment before being rechallenged in their left flank with an injection of 5×10⁶ A20 cancer cells. As a control naïve mice were also challenged with an injection of 5×10⁶ A20 cancer cells (C). Only mice cleared by the treatments acquired an adaptive immune response preventing the formation of new tumors. (A-C) Tumor volume was calculated by multiplying the largest measure by the square of the perpendicular measure divided by two (solid lines=cleared mice, doted lines=non-cleared mice).

FIG. 18 is a graph showing that the synthetic therapeutic bacteriophage can display a functional cytosine deaminase and produce anti-tumoral drug 5-FU. The convertion of 5-FC in 5-FU was measured by absorbance at 255 nm and 290 nm in a spectrophotometer using quartz cuvettes. The concentration of 5-FC and 5-FU was next obtained using the following formula, which is based on the absorbance spectrum of each molecule: [5-FC]=0.119×A290−0.025×A255 and [5-FU]=0.185×A255−0.049×A290.

FIG. 19 is a graph showing that the 5-FU converted by the synthetic therapeutic bacteriophage displaying the cytosine deaminase has an anti-proliferative effects on cancer cells. A20 cancer cells were incubated for 42 h with either the vehicle (PBS 12% DMSO), 200 μM of 5-FC, or the convertion products of either the control bacteriophage (pTAT002) or the cytosine deaminase displaying bacteriophage (pTAT022) after a 24 hour incubaction with 200 μM of 5-FC. Cancer cell death was then monitored by trypan blue coloration. Cancer cell death was only observed with the 5-FU produced by the synthetic bacteriophage displaying the cytosine deaminase.

FIGS. 20A-20B are graphs showing that an alternative start codon GTG improves therapeutic protein display and integrity at the surface of the synthetic therapeutic bacteriophage. (A) Synthetic bacteriophage displaying the anti-PDL1 nanobody production measured through anti-pVIII ELISA assay when cloned with an ATG or a GTG as a start codon. (B) Integrity of nbPDL1-pIII measured by western blot on phage preparation derived from expression systems in which the start codon is either ATG or GTG. The complete form of the fusion protein is indicated with an arrow.

FIGS. 21A-21C are graphs showing that the live biotherapeutic can be engineered to produce bacteriophage particle displaying two or more therapeutic proteins. (A) Bacteriophage production after overnight growth at 37° C. in LB broth for live biotherapeutic secreting a control bacteriophage with no protein displayed (pTAT004+pTAT002), bacteriophages displaying the anti-PD-L1 nanobody on pIII (pTAT032), the gpLX deficient mutant displaying the anti-PD-L1 nanobody on pIII (pTAT032AgpIX) or the double display with the anti-PD-L1 nanobody on pIII+the anti-CTLA-4 anticalin on pIX (pTAT032AgpIX+pTAT035) as measured by an anti-PVIII sandwich ELISA. (B) HRP signal of an ELISA quantifying binding of different phage preparation on PDL1 at the surface of A20 cells. PEG precipitations were either performed on LB (no bacteriophages), bacteriophages derived from pTAT002+pTAT004 (control no-display), bacteriophages derived from pTAT032 (anti-PD-L1 nanobody on pIII) and bacteriophages derived from pTAT032AgpIX+pTAT035 (anti-PD-L1 nanobody on pIII+anti-CTLA-4 anticalin on pIX). Signal was measured using an anti-pVIII-HRP antibody to detect bacteriophage particle bound to the A20 cells. (C) HRP signal of an ELISA quantifying binding of different phage preparation CTLA-4 immobilized in the wells. PEG precipitations were either performed on LB (No bacteriophages), bacteriophages derived from pTAT002+pTAT004 (control no-display), bacteriophages derived from pTAT032 (anti-PD-L1 nanobody on pIII) and bacteriophages derived from pTAT032AgpIX+pTAT035 (anti-PD-L1 nanobody on pIII+anti-CTLA-4 anticalin on pIX). Signal was measured using an anti-HA-HRP antibody to detect the presence of anti-PD-L1 nanobody fused to HA on pIII or HA fused to pIII on the tail of the bacteriophage particles.

FIGS. 22A-22B are graphs showing that live biotherapeutic can secrete synthetic therapeutic bacteriophages displaying a mix of therapeutic proteins on pIII. Live biotherapeutics secreting synthetic bacteriophages displaying nanobodies on pIII against PD-L1 (pTAT032), or CTLA-4 (pTAT019), or both PD-L1 and CTLA-4 (pTAT032+pTAT019) were tested by ELISA assay for their binding activities on PD-L1 (A) or on CTLA-4 (B).

### DETAILED DESCRIPTION OF EMBODIMENTS

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

The expression “degree or percentage of sequence homology” refers herein to the degree or percentage of sequence identity between two sequences after optimal alignment. Percentage of sequence identity (or degree of identity) is determined by comparing two aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity.

As used herein, the term “isolated” refers to nucleic acids or polypeptides that have been separated from their native environment, including but not limited to virus, proteins, glycoproteins, peptide derivatives or fragments or polynucleotides. For example the expression “isolated nucleic acid molecule” as used herein refers to a nucleic acid substantially free of cellular material or culture medium when produced by recombinant DNA techniques, or chemical precursors, or other chemicals when chemically synthesized. An isolated nucleic acid is also substantially free of sequences, which naturally flank the nucleic acid (i.e. sequences located at the 5? and 3? ends of the nucleic acid) from which the nucleic acid is derived.

Two nucleotide sequences or amino-acids are said to be “identical” if the sequence of nucleotide residues or amino-acids in the two sequences is the same when aligned for maximum correspondence as described below. Sequence comparisons between two (or more) peptides or polynucleotides are typically performed by comparing sequences of two optimally aligned sequences over a segment or “comparison window” to identify and compare local regions of sequence similarity. Optimal alignment of sequences for comparison may be conducted by the local homology algorithm of Smith and Waterman, Ad. App. Math 2: 482(1981), by the homology alignment algorithm of Needleman and Wunsch, J. Mol. Biol. 48: 443 (1970), by the search for similarity method of Pearson and Lipman, Proc. Natl. Acad. Sci. (U.S.A.) 85: 2444 (1988), by computerized implementation of these algorithms (GAP, BESTFIT, FASTA, and TFASTA in the Wisconsin Genetics Software Package, Genetics Computer Group (GCG), 575 Science Dr., Madison, Wis.), or by visual inspection. Other alignment programs may also be used such as: “Multiple sequence alignment with hierarchical clustering”, F. CORPET, 1988, Nucl. Acids Res., 16 (22), 10881-10890.

In some embodiments, the present technology relates to an isolated nucleic acid molecule having at least about 75%, or at least about 80%, or at least about 85%, at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity to the nucleic acid sequences described herein.

Unless otherwise defined, scientific and technical terms used in connection with the present invention shall have the meanings that are commonly understood by those of ordinary skill in the art, further unless otherwise required by context, singular terms shall include pluralities and plural terms shall include singular. Generally, nomenclature utilized in connection with and techniques of cell and tissue culture, molecular biology and protein and oligo- or polypeptide chemistry and hybridization described herein and those well-known and commonly used in the art. Standard techniques are used for recombinant DNA, oligonucleotide synthesis, and tissue culture and transformation (e.g., electroporation, lipofectin). Enzymatic reactions and purification techniques are performed according to manufactures specifications or as commonly accomplished in the art as described herein. The foregoing techniques and procedures are generally performed according to the conventional methods well known in the art and as described herein in various general and more specific references that are cited and discussed throughout the present specification. (See, e.g., Sambrook et al., Molecular Cloning. A Laboratory Manual).

The term “antibody”, as used herein, refers to immunoglobulin molecules and immunologically active portions of immunoglobulin molecules, i.e., molecules that contain an antigen-binding site which specifically binds (“immunoreacts with”) an antigen. Structurally, the simplest naturally occurring antibody (e.g., IgG) contains four polypeptide chains, two heavy (H) chains and two light (L) chains inter-connected by disulfide bonds. The immunoglobulins represent a large family of molecules that include several types of molecules, such as IgD, IgG, IgA, IgM and IgE.

As used herein, the term “bispecific antibody” refers to an artificial protein that is composed of fragments of two different monoclonal antibodies and consequently binds to two different types of antigen.

The term “immunoglobulin molecule” includes, for example, hybrid antibodies, or altered antibodies, and fragments thereof.

“Antigen” as used herein refers to a substance that is recognized and bound specifically by an antibody. Antigens can include, for example, peptides, proteins, glycoproteins, polysaccharides and lipids; equivalents and combinations thereof. As used herein, the term “surface antigens” refers to the plasma membrane components of a cell and encompasses the integral and peripheral membrane proteins, glycoproteins, polysaccharides and lipids that constitute the plasma membrane. An “integral membrane protein” is a transmembrane protein that extends across the lipid bilayer of the plasma membrane of a cell. A typical integral membrane protein contains at least one “membrane spanning segment” that generally comprises hydrophobic amino acid residues. Peripheral membrane proteins do not extend into the hydrophobic interior of the lipid bilayer and are bound to the membrane surface by noncovalent interaction with other membrane proteins.

“Antibody fragments” include a portion of an intact antibody, preferably with the antigen binding or variable region of the intact antibody. Examples of antibody fragments include, but are not limited to, Fab, Fab′, F(ab′)2, and Fv fragments; diabodies; linear antibodies (See Zapata et al., Protein Eng. 8(10): 1057-1062 (1995)); single-chain antibody molecules; and multispecific antibodies formed from antibody fragments.

A single-chain variable fragment (scFv) is typically a fusion protein of the variable regions of the heavy (VH) and light chains (VL) of immunoglobulins that are connected with a short linker peptide of 10 to about 25 amino acids. The linker is usually rich in glycine for flexibility, as well as serine or threonine for solubility. The linker can either connect the N-terminus of the VH with the C-terminus of the VL, or vice versa.

As used herein, “bacteriophage” refers to a virus that infects bacteria. Similarly, “archaeophage” refers to a virus that infects archaea. The term “phage” is used herein to refer to both types of viruses but, in certain instances, as indicated by the context may also be used as shorthand to refer to a bacteriophage or archaeophage specifically. Bacteriophage and archaeophage are obligate intracellular parasites (with respect to both the step of identifying a host cell to infect and to only being able to productively replicate their genome in an appropriate host cell) that infect and multiply inside bacteria/archaea by making use of some or all of the host biosynthetic machinery. Though different bacteriophages and archaeophages may contain different materials, they all contain nucleic acids and proteins, and can, under certain circumstances, be encapsulated in a lipid membrane.

Depending upon the phage, the nucleic acid may be either DNA or RNA (but typically not both) and it can exist in various forms, with the size of the nucleic acid depending on the phage. The simplest phage only have genomes a few thousand nucleotides in size, while the more complex phages may have more than 100,000 nucleotides in their genome, and, in rare instances, more than 1,000,000. Additionally, phages may be covered by a lipid membrane and may also contain different materials. The number of different kinds of protein and the amount of each kind of protein in the phage particle will vary depending upon the phage. The proteins protect the nucleic acid from nucleases in the environment and are functional in infection.

Many filamentous and non-filamentous phage genomes have been sequenced, including, for example, the filamentous phages M13, fl, fd, Ifi, Ike, Xf, Pfl, and Pf3. Within the class of filamentous phages, M13 is the most well-characterized species, as its 3-dimensional structure is known and the functions of its coat proteins are well-understood. Specifically, the M13 genome encodes five coat proteins pIII, VIII, VI, VII, and IX, which are used as sites for the insertion of foreign DNA into the M13 vectors.

As used herein, a “phage genome” includes naturally occurring phage genomes and derivatives thereof. Generally (though not necessarily), derivatives possess the ability to propagate in the same hosts as the parent. In some embodiments, the only difference between a naturally occurring phage genome and a derivative phage genome is the addition or deletion of at least one nucleotide from at least one end of the phage genome (if the genome is linear) or along at least one point in the genome (if the genome is circular).

As used herein, a “host cell” or the like is a cell that can form phage from a particular type of phage genomic DNA. In some embodiments, the phage genomic DNA is introduced into the cell by infection of the cell by a phage. The phage binds to a receptor molecule on the outside of the host cell and injects its genomic DNA into the host cell. In some embodiments, the phage genomic DNA is introduced into the cell using transformation or any other suitable techniques. In some embodiments, the phage genomic DNA is substantially pure when introduced into the cell. The phage genomic DNA can be present in a vector when introduced into the cell. By way of non-limiting example, the phage genomic DNA is present in a yeast artificial chromosome (YAC) that is introduced into the phage host cell by transformation or an equivalent technique. The phage genomic DNA is then copied and packaged into a phage particle following lysis of the phage host cell.

As used herein, “outer-surface sequences” refer to nucleotide sequences that encode “outer-surface proteins” of a genetic package. These proteins form a proteinaceous coat that encapsulates the genome of the genetic package. Typically, the outer-surface proteins direct the package to assemble the polypeptide to be displayed onto the outer surface of the genetic package, e.g. a phage or bacteria.

An “inducible promoter” refers to a regulatory region that is operably linked to one or more genes, wherein expression of the gene(s) is increased in the presence of an inducer of said regulatory region or increased in the absence of repressor of said regulatory region. An inducible promoter can be induced by exogenous environmental condition(s), which refers to setting(s) or circumstance(s) under which the promoter described herein is induced. Exogenous environmental conditions refer to the environmental conditions external to the intact (unlysed) engineered microorganism, endogenous or native to tumor environment, or the host subject environment, or to exogenously introduced perturbations to the environment. Inducible promoters can comprise one or more regulatory elements, which include, but are not limited to, enhancer sequences, response elements, protein recognition sites, inducible elements, promoter control elements, protein binding sequences, 5′ and 3′ untranslated regions, transcriptional start sites, termination sequences, polyadenylation sequences, riboswitches and introns.

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

A solution to treat cancers by acting on several therapeutic targets simultaneously is to use a molecular scaffold capable of coupling several therapeutic molecules. Filamentous bacteriophages are large immunogenic biological structures upon which therapeutic proteins or peptides can be displayed. The combination of the immunogenic activity of filamentous bacteriophages with therapeutic proteins, or peptides, could thus improve the efficacy of cancer treatments. Furthermore, filamentous bacteriophage can be secreted by bacteria, providing an efficient way to deliver the drug locally at tumor sites.

According to various embodiments, the present technology relates to an operable synthetic therapeutic bacteriophages live biotherapeutic capable of delivering synthetic therapeutic bacteriophages.

According to some embodiments, the present technology relates to an operable live biotherapeutic capable of delivering synthetic therapeutic bacteriophages for the treatment of cancers. In some implementations, the synthetic therapeutic bacteriophages is delivered by a live biotherapeutic bacterium. In some instances, the synthetic bacteriophage is immunogenic and displays mono- or multi-specific therapeutic proteins.

In some embodiments, the present disclosure provides a live biotherapeutic for the delivery of synthetic therapeutic bacteriophages.

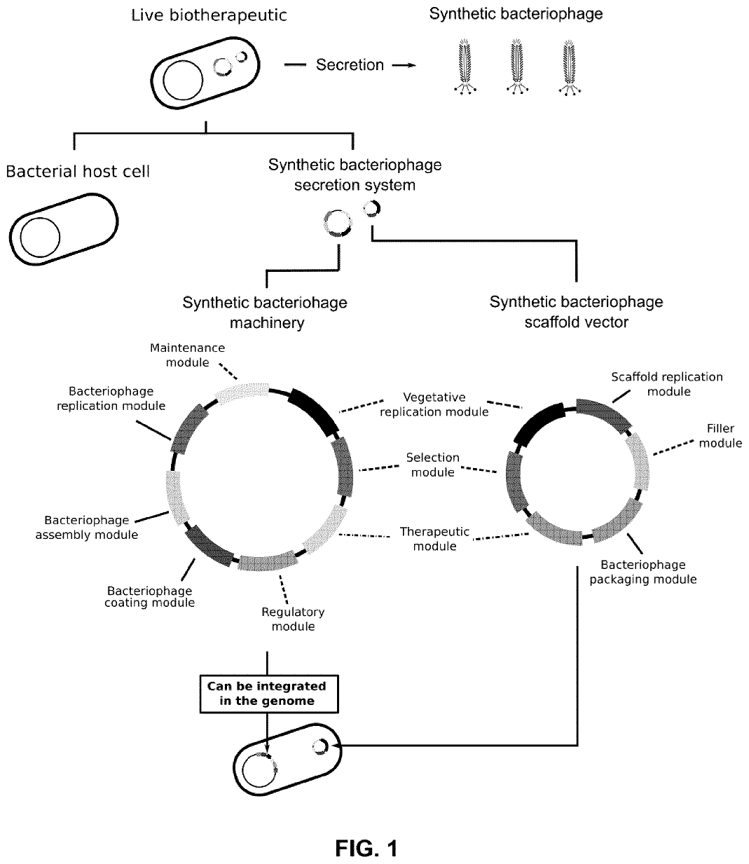

In some embodiments, the present technology relates to a bacterial host engineered with a synthetic bacteriophage secretion system composed of the synthetic bacteriophage machinery and the synthetic bacteriophage scaffold vector (FIG. 1). The synthetic bacteriophage machinery is responsible for the replication and the assembly of the synthetic therapeutic bacteriophages. The synthetic bacteriophage scaffold vector serves as template to produce the nucleic acid scaffold for the assembly of the synthetic therapeutic bacteriophages.

In some embodiments, the bacterial host engineered to deliver the synthetic therapeutic bacteriophages can be derived from anyone of the following: the Enterobacteriaceae family (Citrobacter sp., Enterobacillus sp., Enterobacter sp., Escherichia sp., Klebsiella sp., Salmonella sp., Shigella sp.), from the Pseudomonadaceae family (Pseudomonas sp.) and from the Vibrionaceae family (Vibrio sp.). In some other embodiments, the bacterial engineered to deliver the therapeutic bacteriophages is an attenuated form derived from anyone of the Enterobacteriaceae family (Citrobacter sp., Enterobacillus sp., Enterobacter sp., Escherichia sp., Klebsiella sp., Salmonella sp., Shigella sp.), from the Pseudomonadaceae family (Pseudomonas sp.) and from the Vibrionaceae family (Vibrio sp.). In another embodiment the bacterial host is pathogenic tumor targeting bacteria such as, but not limited to, Salmonella typhimurium, Salmonella choleraesuis, Vibrio cholera. In yet another embodiment, the bacterial host is a non-pathogenic bladder colonizing bacteria such as, but not limited to, Escherichia coli 83972, Escherichia coli HU2117. In yet another embodiment, the bacterial host is a non-pathogenic tumor targeting bacteria such as, but not limited to, Escherichia coli Nissle 1917, Escherichia coli MG1655.

In yet another embodiment, the bacterial host is a tumor targeting bacteria such as, but not limited to, Escherichia coli.

The bacterial host engineered to secrete the therapeutic bacteriophages can be biocontained to prevent its dissemination in the environment. The biocontainment can be achieved by disrupting essential genes to render the bacterial host auxotroph. In a non-limiting example, auxotroph bacterial hosts can be engineered by disrupting the gene dapA or thyA, which respectively renders the bacterial host dependent on exogenous source of Diaminopimelic acid (DAP) or thymine. In an embodiment, the bacterial cell is biocontained using a single biocontainment strategy disrupting a single essential gene (e.g. only DAP auxotrophy or thymine auxotrophy). In yet another embodiment, the bacterial cell is biocontained by the disruption of two or more essential genes (e.g. DAP auxotrophy and thymine auxotrophy). Essential genes that can be disrupted to generate auxotroph E. coli bacterial host include, but are not limited to, yhbV, yagG, hemB, secD, secF, ribD, ribE, ML, drs, ispA, dnaX, adk, hemH, IpxH, cysS, fold, rplT, infC, thrS, nadE, gapA, yeaZ aspS, argS, pgsA, yejM, metG, folE, yejM, gyrA, nrdA, nrdB, folC, accD, fabB, gltX, gA, zipA, dapE, dapA, der, hisS, ispG, suhB, tadA, acpS, era, rnc, ftsB, eno, pyrG, chpR, Igt, ba, pgk, yqgD, metK yqgF, plsC, ygiT, pare, ribB, cca, ygjD, tdcF, yraL yihA ftsN, murl, murB, birA, secE, nusG, rplJ, rplL, rpoB, rpoC, ubiA, plsB, lexA, dnaB, ssb, alsK groS, psd, orn, yjeE, rpsR, chpS, ppa, valS, yjgP, yjgQ, dnaC, rnbF, IspA, ispH, dapB, folA, imp, yabQ, ftsL, ftsl, murE, murF, mraY, murD, ftsW murG, murC, fssQ, ftsA, ftsZ, IpxC, secM, secA, can, folK, hemL, yadR, dapD, map, rpsB, infB, nusA, ftsH, obgE, rpmA, rplU, ispB, murA, yrbB, yrbK, yhbN, rpsl, rplM, degS, mreD, mreC, mreB, accB, accC, yrdC, def, fmt, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsJ, fusA, rpsG, rpsL, trpS, yrfF, asd, rpoH, ftsX, ftsE, ftsY, frr, dxr, ispU, rfaK, kdtA, coaD, rpmB, dfp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR rplF, rpsH, rpsN, rplE, rplX rplN, rpsQ, rpmC, rplP, rpsC, rplV, rpsS, rplB, cdsA, yaeL, yaeT, IpxD, fabZ IpxA, IpxB, dnaE, accA, tiLS, proS, yafF, tsf, pyrH, olA, ripB, leuS, Int, glnS, fldA, cydA, infA, cydC, ftsK, lolA, serS, rpsA, msbA, IpxK, kdsB, mukF, mukE, mukB, asnS, fabA, mviN, rne, yceQ, fabD, fabG, acpP, tmk, holB, lolC, lolD, lolE, purB, ymfK, minE, mind, pth, rsA, ispE, lolB, hemA, prfA, prmC, kdsA, topA, ribA, fabl, racR, dicA, ydfB, tyrS, ribC, ydiL, pheT, pheS, yhhQ, bcsB, gyQ, yibJ, gpsA, and their functional homologs

The bacterial host can be genetically engineered to be protease deficient as a mean to increase the production, and the secretion, of the therapeutic bacteriophages. In some embodiments, the bacterial host is deficient for one or more proteases. In some other embodiments, the bacterial host is deficient for the ompT gene which encodes the protease 7 in E. coli. In another embodiment, the bacterial host is deficient for the Ion gene, which encodes the Lon protease in E. coli. In yet another embodiment, the bacterial host is deficient for the ompT gene and for the sulA gene, which encodes the cell division inhibitor sulA, allowing cells to divide more normally in the absence of protease. In yet another embodiment, the bacterial host is deficient for the Ion gene and for the sulA gene. In yet another embodiment, the bacterial host is deficient for the Ion gene, the ompT gene, and for the sulA gene.

The bacterial host can be genetically engineered to be attenuated and evade the human immune system. Immune cells recognize the LPS displayed on the outer membrane of bacteria and eliminate them. Strategies to manipulate the structure of LPS have been developed to evade the immune system and to extend the half-life of bacteria injected in the bloodstream and LPS modification that allow bacteria to evade the immune system are well documented (Motohiro Matsuura, Front Immunol., 2013, 4:019; Steimle et al., Int. J. Med. Microbiol., 2016 306:290; Simpson et al., Nat. Rev. Microbiol., 2019, 17:403; incorporated herein by reference) that we quote in their entirety. It thus possible to truncate LPS or manipulate their biosynthesis pathway to decrease the immunogenicity of the modified bacterium. Therefore, in some embodiment the bacterial host possesses altered, or truncated, LPS in order to escape the immune system.

In some embodiments, the synthetic bacteriophage machinery comprises of a bacteriophage assembly module, a bacteriophage replication module, a bacteriophage coating module, and a therapeutic module.

In some embodiments, the bacteriophage assembly module is responsible for the assembly of the bacteriophage coating proteins onto the bacteriophage ssDNA scaffold. The Bacteriophage assembly module can include, but is not limited to, the bacteriophage gene gpI, encoding the proteins pI and pXI, and the bacteriophage gene gpIV, encoding for the protein pIV. In an embodiment, some or all the genes encoding pI, pXI and pIV can be derived from one or more of the closely related filamentous bacteriophages belonging to the Inoviridae family such as, but not limited to, bacteriophages M13, Fd, F1, Ifl, Ike, Pfl, Pf3, fs-2, and B5. In another embodiment, the genes encoding pI, pXI and pIV can be derived from the filamentous bacteriophage M13.

In some embodiments, the bacteriophage replication module is responsible for the replication of the bacteriophage ssDNA scaffold. It encodes proteins that recognize the scaffold replication module located on the synthetic bacteriophage scaffold vector and trigger a rolling circle replication producing cyclized ssDNA scaffold molecules. The bacteriophage replication module can include, but is not limited to, the bacteriophage gene gpII, encoding the proteins pII and pX, and the bacteriophage gene gpV, encoding the protein pV. In an embodiment, some or all the genes encoding pII, pX and pV can be derived from one or more of the closely related bacteriophages belonging to the Inoviridae family such as, but not limited to, bacteriophages M13, Fd, F1, Ifl, Ike, Pfl, Pf3, fs-2, and B5. In another embodiment, the genes encoding pII, pX and pV can be derived from the filamentous bacteriophage M13.

In some embodiments, the bacteriophage coating module comprises coating proteins that assemble onto the bacteriophage ssDNA scaffold to form the bacteriophage. The bacteriophage coating module can include, but is not limited to, the bacteriophage genes gpIII, gpVI, gpVII, gpVIII, and gpIX, or a portion thereof, respectively coding for protein pIII, pVI, pVII, pVIII, and pIX or coding for a portion thereof. In some embodiments, one or more coating genes present in the bacteriophage coating module can also be present in the therapeutic module where they are fused to one, or more, a therapeutic protein. In some other embodiments, when one or more coating genes are present in the therapeutic module and fused to one or more therapeutic proteins, the corresponding coating genes are not present in the bacteriophage coating module. In an embodiment, some or all the genes encoding pIII, pVI, pVII, pVIII, and pIX can be derived from one or more of the closely related bacteriophages belonging to the Inoviridae family such as, but not limited to, bacteriophages M13, Fd, F1, Ifl, Ike, Pfl, Pf3, fs-2, and B5. In another embodiment, the genes encoding pIII, pVI, pVII, pVIII, and pIX can be derived from the filamentous bacteriophage M13.

In some embodiments, the therapeutic module comprises the therapeutic protein to be displayed by the therapeutic bacteriophage. The therapeutic module comprises, but is not limited to, one or more bacteriophage coating protein gene, fused to one or more therapeutic proteins. The bacteriophage therapeutic module can include, but is not limited to, the bacteriophage coating genes gpIII, gpVI, gpVII, gpVIII, and gpIX, respectively coding for protein pIII, pVI, pVII, pVIII, and pIX, fused to one, or more, therapeutic proteins. In an embodiment, some or all the genes encoding pIII, pVI, pVII, pVIII, and pIX can be derived from one or more of the closely related bacteriophages belonging to the Inoviridae family such as, but not limited to, bacteriophages M13, Fd, F1, Ifl, Ike, Pfl, Pf3, fs-2, and B5. In another embodiment, the genes encoding pIII, pVI, pVII, pVIII, and pIX can be derived from the filamentous bacteriophage M13. The therapeutic protein to be displayed by the bacteriophage can be fused to any of the phage coating proteins such as pIII, pVI, pVII, pVIII, and pIX.

In some embodiments the therapeutic protein is fused to a mutant pVIII coating protein that improves the display of large protein on the surface of the synthetic bacteriophage. pVIII mutant proteins that improve the display of large protein on the surface of filamentous bacteriophages have been identified (S. Sidhu et al. J. Mol. Biol. (2000) 296, 487-495; incorporated herein by reference). In some embodiment the therapeutic protein is displayed on a pVIII coating protein identified using an approach similar to S. Sidhu et al. In some embodiment, the therapeutic protein is displayed on a pVIII coating protein corresponding to the pVIII(1a) mutant described in S. Sidhu et al. Mol. Biol. (2000) 296, 487-495. In another embodiment, the therapeutic protein is displayed on a pVIII coating corresponding to the pVIII(2e) mutant described in S. Sidhu et al. Mol. Biol. (2000) 296, 487-495. In yet another embodiment, the therapeutic protein is displayed on a pVIII coating corresponding to the pVIII(2f) mutant described in S. Sidhu et al. Mol. Biol. (2000) 296, 487-495.

In some instances, expressing too much of a therapeutic protein can have a detrimental effect on the bacterial host, which in the end results in poor synthetic bacteriophage secretion. Alternative start codons rely on the start tRNA wobble that allows the start of transcription on the wrong set of nucleotides. This stalls ribosomes and might allow for improved ribosome trafficking on the gene, thus producing more complete protein products. Several codons can be used as alternative start codon (Hecht et al. Nucleic Acids Research, 2017, Vol. 45, No. 7 3615-3626; incorporated herein by reference). In some embodiment the therapeutic protein start codon is any of the 64 codons. In some embodiments the traduction of the therapeutic protein gene starts on the standard start codon ATG. In some other embodiment the therapeutic protein start codon is TUG. In yet another embodiment the therapeutic protein start codon is GTG.

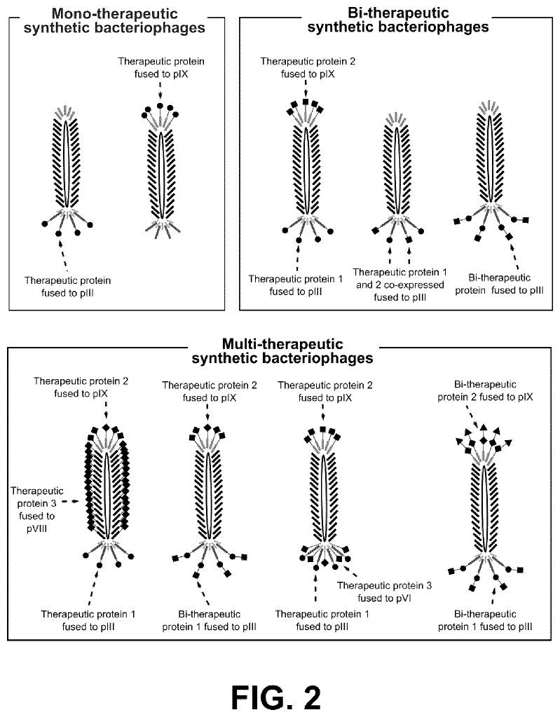

In some embodiments, the therapeutic protein is fused to the N- or C-terminal end of the coating protein. In some other embodiments, the therapeutic protein is fused within the coating protein by insertion in any part of the protein. In some other embodiments, the coating protein is fused to the therapeutic protein using one or more protein tags such as, but not limited to, human influenza hemagglutinin (HA-tag), poly-histidine tag (His-tag), FLAG-tag, or myc-tag. In yet another embodiment, the therapeutic protein is fused to the coating protein using an amino acid linker sequence. The linker can be flexible, rigid, or cleavable as described by Chen et al. (Adv Drug Deliv Rev. 2013 Oct. 15; 65(10): 1357-1369; incorporated herein by reference). In yet another embodiment, the linker can also include tag sequences such as, but not limited to, human influenza hemagglutinin (HA-tag), poly-histidine tag (His-tag), FLAG-tag, or myc-tag. The one or more therapeutic proteins are fused to the one or more coating proteins pIII, pVI, pVII, pVIII, and pIX in ways that are not detrimental for the activity of the therapeutic protein and for bacteriophage assembly. In some embodiment, the one or more therapeutic proteins are fused to full length coating proteins. In another embodiment, the one or more therapeutic proteins can be fused to full length coating proteins via one or more linker sequences. In another embodiment, the one or more therapeutic proteins can be fused to truncated coating proteins comprising domains essential for bacteriophage assembly. In yet another embodiment, the one or more therapeutic proteins can be fused to truncated coating proteins comprising domains essential for bacteriophage assembly via one or more linker sequences. In some embodiments, where the one or more therapeutic proteins are fused to the N-terminus of the coating proteins, the fusion protein further comprises a leader peptide sequence at its N-terminus end to ensure the translocation of the protein to the bacterial outer membrane for phage assembly. In some embodiments, the leader peptide sequences include, but is not limited to one or more leader peptides from DsbA, PelB, TorA, and PhoA signal peptides. In yet another embodiment, the leader peptide is an optimized DsbA and PelB signal peptide with improved translocation activity as described by Han et al. (Han et al. AMB Expr (2017) 7:93; incorporated herein by reference). In yet another embodiment the leader peptide is the signal peptide from BKC-1 described by Bharathwaj et al. (Bharathwaj et al. mBio. 2021 Jun. 29; 12(3); incorporated herein by reference). In some embodiment, the leader peptide is from PelB. When multi-therapeutic bacteriophages are to be secreted, two, or more, therapeutic proteins are fused to one or more of the coating proteins (FIG. 2). Multi-therapeutic bacteriophages can also comprise a bacteriophage displaying one or more therapeutic proteins fused together. In some embodiment, the therapeutic proteins are fused using one or more protein tags such as, but not limited to, human influenza hemagglutinin (HA-tag), poly-histidine tag (His-tag), FLAG-tag, and myc-tag. In another embodiment, the therapeutic proteins are fused using an amino acid linker sequence. The linker can be flexible, rigid, or cleavable as described by Chen et al., 2013 (Adv Drug Deliv Rev. 2013 Oct. 15; 65(10): 1357-1369. Fusion Protein Linkers: Property, Design and Functionality; incorporated herein by reference). In yet another embodiment, the linker can also include tag sequences such as, but not limited to, human influenza hemagglutinin (HA-tag), poly-histidine tag (His-tag), FLAG-tag, and myc-tag. The one, or more, therapeutic proteins fused to the one, or more, bacteriophage coating proteins can be any mono- or multi-specific binding proteins comprise, but not limited to, fragments antigen binding (Fab and F(ab′)2), single-chain variable fragments (scFv), di-single-chain variable fragments (di-scFv), bi-specific T-cell engager (BiTE), TCR, soluble TCR, single-chain T cell receptors variable regions (scTv), single-domain antibodies (Nanobodies), lipocalins (Anticalins), monobodies (Adnectins), affibodies, affilins, affimers, affitins, alphabodies, Armadillo repeat protein-based scaffolds, aptamers, atrimers, avimers, DARPins, fynomers, knottins, Kunitz domain peptides, and adhesins. Binding proteins also includes extracellular domains of receptors and their ligands such as, but not limited to, PD-1, PD-L1, CTLA-4, B7-1, B7-2, CD112, CD155, TIGIT, CD96, CD226, CD112R, CD96, CD111, CD272, B7H4, CD28, CD80, CD86, OX40, OX40-L, ICOS, ICOS-LG, CD137, CD137-L, AITR, AITR-L, CD27, CD70, TNF-α, TNFR1, TNFR2, LAG-3, TIM-3, galectin-9.

In another embodiment, the one or more therapeutic proteins fused to the one, or more, bacteriophage coating proteins are peptides.

In yet another embodiment, the one, or more, therapeutic proteins fused to the one, or more, bacteriophage coating proteins are enzymes.

In some other embodiment, the one or more therapeutic proteins fused to the one, or more, bacteriophage coating proteins are a combination of binding proteins and peptides, or of binding proteins and enzymes, or of enzymes and peptides, or of binding proteins, enzymes, and peptides.

In some embodiment, the synthetic bacteriophage machinery can include optional modules such as: a regulatory module comprising regulatory elements controlling the activity of the synthetic bacteriophage machinery and a synthetic bacteriophage scaffold vector. As a non-limiting example, the regulatory module can turn on or off some, or all, the genes of the bacteriophage machinery, and/or of the synthetic bacteriophage scaffold vector. Turning off the bacteriophage machinery, and/or the synthetic bacteriophage scaffold vector, during phases of large-scale production of the live biotherapeutic can be advantageous to avoid selection pressure and evolution drifting. The regulatory module, when present in the bacteriophage machinery, can include one or more genes and regulatory elements encoding one or more proteins or non-coding RNAs capable of regulating the expression of genes, or capable of being used to regulate the expression of genes, of the synthetic bacteriophage machinery and/or of the synthetic bacteriophage scaffold vector (e.g., transcription factors, activators, repressors, riboswitches, CRISPR-Cas9, Zinc Finger Nucleases (ZFN), TALEs, and taRNAs).

In some embodiment, the synthetic bacteriophage machinery can include optional modules such as: a maintenance module which includes a replication machinery capable of recognizing the origin of replication (oriP) of vectors bearing a vegetative replication module, such as the synthetic bacteriophage scaffold vector or vectors bearing modules of the synthetic bacteriophage machinery. The maintenance module is needed when the oriV of vegetative modules are not compatible with the replication machinery of the bacterial host. The maintenance module allows the replication of any plasmids comprising a vegetative replication module compatible with its replication machinery. The maintenance module can be heterologous to the bacterial host. When the maintenance of vectors needs to be restricted to the donor bacterium, it may be preferable to locate (e.g., integrate) the maintenance module into the donor bacterium chromosome. Alternatively, the maintenance module may be located on one or more vectors. The maintenance module can also comprise one or more genes and regulatory elements responsible for adequate DNA partitioning.

In some embodiment, some or all the promoters controlling the expression of the genes of the phage machinery are inducible promoters. In another embodiment, some or all the promoters controlling the expression of the genes of the phage machinery are inducible promoters induced by one or more exogenous molecules such as, but not limited to, L-arabinose, rhamnose, IPTG, and tetracycline. In yet another embodiment, some or all the promoters controlling the expression of the genes of the phage machinery are inducible promoters induced by one or more exogenous environmental conditions of the tumor microenvironment such as, but not limited to, low oxygen levels (hypoxia), acidic pH (<7), oxidative condition (high level of H2O2). In yet another embodiment, some or all the promoters controlling the expression of the genes of the bacteriophage machinery are induced by one or more exogenous molecules and/or by one or more exogenous environmental conditions present in the tumor microenvironment. In another embodiment, some or all the promoters controlling the expression of the genes of the bacteriophage machinery are induced by one or more molecule produced by the host bacteria such as diaminopimelic acid and N-acyl-homoserine lactone.

In some embodiments, the synthetic bacteriophage machinery is integrated in the genome of the bacterial cell. In another embodiments, some, or all, the modules of the synthetic bacteriophage machinery are located onto the synthetic bacteriophage scaffold vector. In yet another embodiments, some, or all, the modules of the synthetic bacteriophage machinery are located onto one or multiple vectors, while some or none of the synthetic bacteriophage machinery modules remain in the genome of the bacterial cell and the synthetic bacteriophage scaffold vector. When modules of the synthetic bacteriophage machinery are located onto vectors, other than the synthetic bacteriophage scaffold vector, two additional modules are present in each of the vectors: a vegetative replication module and a selection module, and one additional module is present either in one or more of the vectors or in the genome of the bacterial host: the maintenance module.

The vegetative replication module allows vectors bearing modules of the synthetic bacteriophage machinery to be replicated into the bacterial host cell. The vegetative replication module comprises an origin of replication oriV compatible with the bacterial host, and/or an oriV compatible with the maintenance module and which can be derived from the bacteriophage M13 and/or one of the following family of bacterial vectors IncA, IncB/O (Inc10), IncC, IncD, IncE, IncF1, IncF2, IncG, IncHI1, IncHI2, IncI1, IncI2, IncJ, IncK, IncL/M, IncN, IncP, IncQ1, IncQ2, IncR, IncS, IncT, IncU, IncV, IncW, IncX1, IncX2, IncY, IncZ, ColE1, ColE2, ColE3, p15A, pSC101, IncP-2, IncP-5, IncP-7, IncP-8, IncP-9, Inc1, Inc4, Inc7, Inc8, Inc9, Inc11, Inc13, Inc14 and/or Inc18. In an embodiment, the vegetative replication module oriV can be derived from one of the ColE1, pSC101, F, p15A, M13 family of bacterial vectors. For example, the vegetative replication module can be derived from the bacterial vector ColE1.

The selection module allows the vector to be stably maintained into the bacterial host. The selection module includes one or more genes conferring a selectable trait for the discrimination of bacteria bearing one or more modules of the bacteriophage machinery. The selection module is operably connected with the one or more bacterial vector of the synthetic bacteriophage secretion system. The selectable trait can be, but is not limited to, an antibiotic resistance gene, a gene coding for a fluorescent protein (including a green fluorescent protein), an auxotrophic selection marker, a gene coding for a β-galactosidase (e.g., the bacterial lacZ gene), a gene coding for a luciferase, a gene coding for a chloramphenicol acetyltransferase (e.g., the bacterial cat gene), a gene coding for an enzyme allowing the use of a nutrient that the bacterial chassis cannot process (e.g. thiA, if the endogenous thiA is removed from the bacterial chromosome), a gene coding for a β-glucuronidase, and regulatory elements responsible for adequate DNA partitioning. In some embodiment, some or all the promoters controlling the expression of the genes of the selection module are inducible promoters. In some embodiments, the minimal synthetic bacteriophage scaffold vector comprises a vegetative replication module, a selection module, a scaffold replication module, and a packaging module.

In some embodiments, the vegetative replication module allows the synthetic bacteriophage scaffold vector to be replicated into the bacterial host cell. The vegetative replication module comprises an origin of replication oriV compatible with the bacterial host, or an oriV compatible with the maintenance module and which can be derived from the bacteriophage M13 and/or one of the following family of bacterial vectors IncA, IncB/O (Inc10), IncC, IncD, IncE, IncF1, IncF2, IncG, IncHI1, IncHI2, IncI1, IncI2, IncJ, IncK, IncL/M, IncN, IncP, IncQ1, IncQ2, IncR, IncS, IncT, IncU, IncV, IncW, IncX1, IncX2, IncY, IncZ, ColE1, ColE2, ColE3, p15A, pSC101, IncP-2, IncP-5, IncP-7, IncP-8, IncP-9, Inc1, Inc4, Inc7, Inc8, Inc9, Inc11, Inc13, Inc14 and/or Inc18. In an embodiment, the vegetative replication module oriV can be derived from one of the ColE1, pSC101, F, p15A, M13 family of bacterial vectors. For example, the vegetative replication module can be derived from the bacterial vector ColE1.

In some embodiments, the scaffold replication module allows the production of the ssDNA synthetic bacteriophage scaffold. The scaffold replication module comprises an origin of replication (oriV) recognized by bacteriophage replication proteins which allows rolling circle replication of the synthetic bacteriophage scaffold vector and the production of cyclized ssDNA synthetic bacteriophage scaffold molecules. The DNA sequence of the bacteriophage oriV can be derived from one of the closely related bacteriophages belonging to the Inoviridae family such as, but not limited to, bacteriophages M13, Fd, F1, Ifl, Ike, Pfl, Pf3, fs-2, and B5. In some embodiment, some or all the promoters controlling the expression of the genes of the scaffold replication module are inducible promoters.

In some embodiments, the bacteriophage packaging module allows the cyclized ssDNA synthetic bacteriophage scaffold to be processed for assembly with the M13 bacteriophage coating proteins. The packaging module comprise comprises a DNA sequence acting as packaging signal to start the assembly of the bacteriophage. The DNA sequence of the packaging signal can be derived from one of the closely related bacteriophages belonging to the Inoviridae family such as, but not limited to, bacteriophages M13, Fd, F1, Ifl, Ike, Pfl, Pf3, fs-2, and B5. In some embodiment, some or all the promoters controlling the expression of the genes of the bacteriophage packaging module are inducible promoters. In some embodiments, a selection module is present and allows the vector to be stably maintained into the bacterial host. The selection module includes one or more genes conferring a selectable trait for identifying bacteria bearing the synthetic bacteriophage scaffold vector. The selection module is operably connected with the synthetic bacteriophage scaffold vector. The selectable trait can be, but is not limited to, an antibiotic resistance gene, a gene coding for a fluorescent protein (including a green fluorescent protein), an auxotrophic selection marker, a gene coding for a β-galactosidase (e.g., the bacterial lacZ gene), a gene coding for a luciferase, a gene coding for a chloramphenicol acetyltransferase (e.g., the bacterial cat gene), a gene coding for a β-glucuronidase, a gene coding for an enzyme allowing the use of a nutrient that the bacterial chassis cannot process (e.g. thiA, if the endogenous thiA is removed from the bacterial chromosome). In some embodiment, some or all the promoters controlling the expression of the genes of the selection module are inducible promoters.

In some embodiments, a filler module is present and allows to change the length of the synthetic bacteriophage. The filler module comprises a random sequence of DNA which only purpose is to change the size of the synthetic bacteriophage scaffold vector and does not necessarily comprise genes or regulatory elements. By changing the size of the scaffold vector, the filler module allows to change the length of the synthetic bacteriophage particles. Having short synthetic bacteriophage can improve the number of secreted particles, since less pVIII coating proteins are needed per bacteriophage. Increasing the size of the synthetic bacteriophage on the other hand allows to increase the distance between therapeutic proteins fused to pIII and pIX, and hence, improves the interaction of these therapeutic proteins with their respective targets. Therefore, in some embodiments the filler module is composed of a DNA sequence which size varies between 0 bp and 100,000 bp of DNA.

In some embodiment, some or all the promoters controlling the expression of the genes of the synthetic bacteriophage scaffold vector are inducible promoters. In another embodiment, some or all the promoters controlling the expression of the genes of the synthetic bacteriophage scaffold vector are inducible promoters induced by one or more exogenous molecules such as, but not limited to, L-arabinose, rhamnose, IPTG, and tetracycline. In yet another embodiment, some or all the promoters controlling the expression of the genes of the synthetic bacteriophage scaffold vector are inducible promoters induced by one or more exogenous environmental conditions of the tumor microenvironment such as, but not limited to, low oxygen levels (hypoxia), acidic pH (<7), oxidative condition (high level of H₂O₂). In yet another embodiment, some or all the promoters controlling the expression of the genes of the synthetic bacteriophage scaffold vector are induced by one or more exogenous molecules and/or by one or more exogenous environmental conditions present in the tumor microenvironment and/or by one or more molecule secreted by the host bacterium.

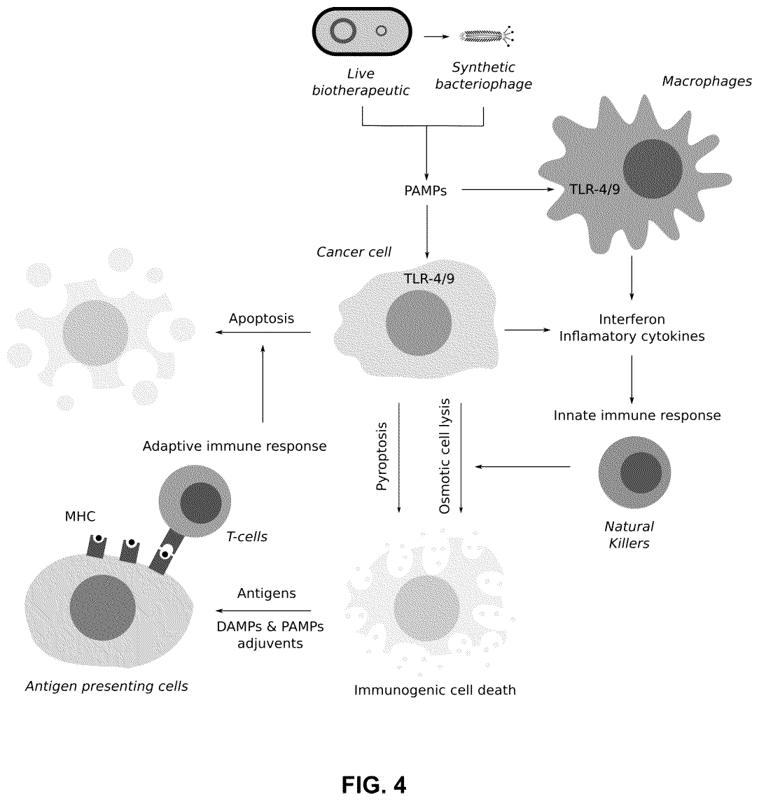

In some embodiments, the synthetic therapeutic bacteriophage stimulates pattern recognition receptors (PRRs). PRRs play a key role in the innate immune response through the activation of pro-inflammatory signaling pathways, stimulation of phagocytic responses (macrophages, neutrophils and dendritic cells) or binding to micro-organisms as secreted proteins. PRRs recognize two classes of molecules: pathogen-associated molecular patterns (PAMPs), which are associated with microbial pathogens and viruses, and damage-associated molecular patterns (DAMPs), which are associated with cell components that are released during cell damage, death, stress, or tissue injury. PAMPS are essential molecular structures required for pathogens survival, e.g., bacterial cell wall molecules (e.g. lipoprotein), bacterial or viral DNA. Some PRRs can be expressed by cells of the innate immune system but other PRRs can also be expressed by other cells (both immune and non-immune cells). PRR are either localized on the cell surface to detect extracellular pathogens or within the endosomes and cellular matrix where they detect intracellular invading viruses. Examples of PRRs include, but are not limited to, Toll-like receptors (TLR1, TLR 2, TLR3, TLR4, TLR5, TLR6, TLR7, TLR8, TLR9, TLR10), C-type lectin receptors (Group I mannose receptors and group II asialoglycoprotein), nucleotide oligomerization (NOD)-like receptors (NODI and NOD2), retinoicacid-inducible gene I (RIG-I)-like receptors (RLR) (RIG-I, MDA5, and DDX3), collectins, pentraxins, ficolins, lipid transferases, peptidoglycan recognition proteins (PGRs) and the leucine-rich repeat receptor (LRR). Upon detection of a pathogen, PRRs activate inflammatory and immune responses mounted against the infectious pathogen. Recent evidence indicates that immune mechanisms activated by PAMPs and DAMPs play a role in activating immune responses against tumor cells as well (Hobohm & Grange, Crit Rev Immunol. 2008; 28(2):95-107, and Krysko D V et al., Cell Death and Disease (2013) 4, e631; here in incorporated as references). Intratumoral injection have been shown to stimulate an immune response with some microorganisms, such as the microorganisms of the disclosure (e.g., bacteria and bacteriophage). In some instances, these have been shown to provide therapeutic benefit in several types of cancers, including solid tumors, melanoma, basal cell carcinomas, and squamous cell carcinoma. The anti-tumoral response observed in those cases is thought to be, in part, due to the proinflammatory properties of the nucleic acid fractions, capsid proteins, and/or cell wall fractions of microorganisms that activate PRRs. The synthetic therapeutic bacteriophage of the present disclosure can naturally trigger an immune response through the presence of PAMPs and DAMPs, which are agonists for PRRs found on immune cells and tumor cells in the tumor microenvironment (see Carroll-Portillo A. et al. Microorganisms. 2019 December; 7(12): 625; herein incorporated as reference) (FIG. 4). Thus, in some embodiments, the synthetic therapeutic bacteriophage of the present disclosure trigger an immune response at the tumor site. In these embodiments, the synthetic therapeutic bacteriophage naturally expresses a PRR agonist, such as one or more PAMPs. Examples of PAMPs are shown in Takeuchi et al. (Cell, 2010, 140:805-820; incorporated herein by reference). In some embodiments, the PRR is DNA from the synthetic bacteriophage which is recognized by immune, or non immune, cells via TLR-9 and/or RIG-I.

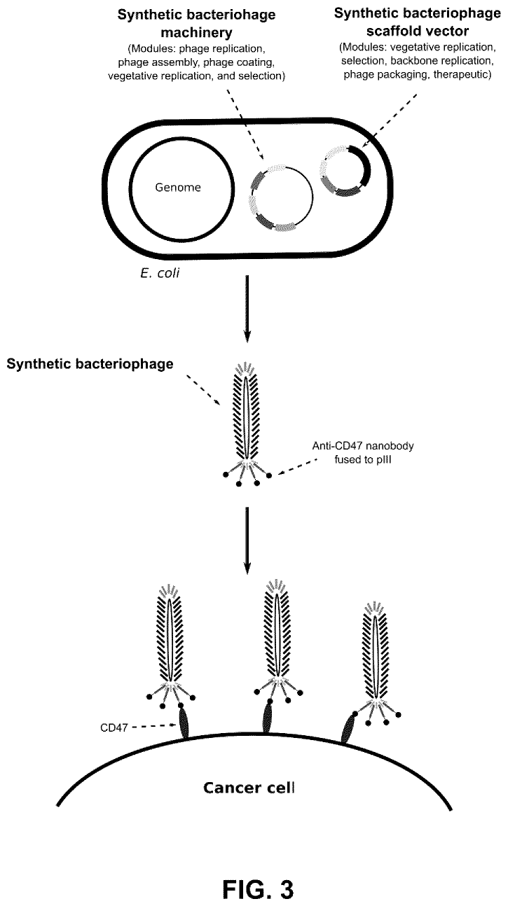

In an embodiment, the therapeutic bacteriophages displays one or more binding proteins that inhibits immune checkpoint (FIG. 3). Several cancer drugs target and inhibit immune checkpoints to activate the immune system and mount an immune response against self-antigens presented by cancerous cells. However, altered immunoregulation can provoke immune dysfunction and lead to autoimmune disorders when administered systemically. The immune dysfunction side effects, e.g., the development of an undesired autoimmune response, can be addressed by delivering an immune checkpoint inhibitor or inhibitor of another immune suppressor molecule locally at the tumor site. The immune checkpoint molecule to be inhibited can be any known or later discovered immune checkpoint molecule or other immune suppressor molecule. In some embodiments, the immune checkpoint molecule, or other immune suppressor molecule, to be inhibited are selected from, but are not limited to, CCR4, CTLA-4, CD80, CD86, PD-1, PD-L1, PD-L2, TIGIT, VISTA, LAG-3, TIM1, TIM3, CEACAM1, LAIR-1, HVEM, BTLA, CD47, SIRPα, CD160, CD200, CD200R, CD39, CD73, B7-H3, B7-H4, IDO, TDO, KIR, and A2aR. When the one or more of the binding proteins are derived from single chain antibodies, their sequence can be, but not limited to, one or more listed in Table 3 and 4 of PCT/US2017/013072; incorporated herein by reference.

In an embodiment, the one or more binding proteins displayed by the therapeutic bacteriophages binds to and inhibit one or more proteins, peptides, or molecule involved in carcinogenesis, the development of cancer, or of metastases. The one or more proteins, peptides, or molecule to be inhibited can be any known or later discovered proteins, peptides, or molecule involved in carcinogenesis, the development of cancer, or of metastases. In some embodiments, the one or more proteins, peptides, or molecule to be inactivated are selected from, but are not limited to, CSF1, CSFIR, CCR4, CCL2, CCL17, CCL22, HER2, GD2, IL-1p, IL-6, IL-10, IL-13, IL-17, IL-27, IL-35, CD20, CD27, CD30, CD33, CD70, TGF-β, M-CSF, EGFR, ERBB2, ERBB3, PGE2, VEGF, VEGFR-2, CXCR4/CXCL12, Tie2, galectin-1, galectin-3, Phosphatidyl serine, and TAM and Tim Phosphatidyl serine receptors.

In an embodiment, the one or more binding proteins displayed by the therapeutic bacteriophages act as agonists to activate co-stimulatory receptor that lead to the elimination of cancerous cells. The one or more co-stimulatory cellular receptors activated by the one or more antibody mimetic can be any known or later discovered co-stimulatory cellular receptors that lead to the elimination of cancerous cells. In some embodiments, the one or more cellular receptor activated by the one or more antibody mimetics are selected from, but not limited to CD40, CD28, ICOS, CD226, CD137, and CD134.

In an embodiment, the one or more binding proteins displayed by the therapeutic bacteriophages are antibody Fc domains triggering antibody-dependent cellular cytotoxicity (ADCC). “ADCC” refer to a cell mediated reaction, in which nonspecific cytotoxic cells that express Fc receptors (FcRs), such as NK cells, recognize bound antibody on a target cell and subsequently cause lysis of the target cell. NK cells are key mediators of ADCC and induce direct cellular cytotoxicity via perforin and granzyme, FasL, and TRAIL interactions as well as cytokine production. NK cell activation required to trigger ADCC can occur via Fc receptors for IgG (FcγRs) (FcγRI, FcγRIIA, and FcγRIIIA in human and FcγRI, FcγRIII, and FcγRIV in mice) that recognize the Fc domain of IgG antibody. Thus, a synthetic therapeutic bacteriophage displaying one or more engineered Fc domains that binds to NK's activating FcγRs can be used to recruit and activate NK at tumor sites to mediate ADCC and eliminate tumor cells. Therefore, in some embodiments, the synthetic therapeutic bacteriophage displays one or more Fc domains that bind to NK's activating FcγRs. The one or more Fc domains can be any known or later discovered Fc domain that activates NK to trigger ADCC. A list of antibodies with Fc domains triggering ADCC can be found in Table 36 from PCT/US2017/013072 (incorporated herein by reference).

In an embodiment, the one or more binding proteins displayed by the therapeutic bacteriophage binds to other binding proteins such as, but not limited to, IgG antibodies, nanobodies, affibodies, anticalins, antibody fragments, ScFV, biotin, streptavidin.

In an embodiment, the synthetic therapeutic bacteriophages display a combination of one or more binding proteins inhibiting immune checkpoints and/or one or more binding proteins that inhibit proteins, peptides, or molecule involved in carcinogenesis, the development of cancer, or of metastases, and/or one or more binding proteins acting as agonists to activate cellular receptors preventing carcinogenesis, the development of cancer, or of metastases, and or one or more Fc domains triggering ADCC and tumor cell elimination. In one embodiment, the binding proteins that can be displayed by the synthetic therapeutic bacteriophages include: fragments antigen binding (Fab and F(ab′)2), single-chain variable fragments (scFv), di-single-chain variable fragments (di-scFv), bi-specific T-cell engager (BiTE), TCR, soluble TCR, single-chain T cell receptors variable regions (scTv), single-domain antibodies (Nanobodies), lipocalins (Anticalins), monobodies (Adnectins), affibodies, affilins, affimers, affitins, alphabodies, Armadillo repeat protein-based scaffolds, aptamers, atrimers, avimers, DARPins, fynomers, knottins, Kunitz domain peptides, and adhesins.

In an embodiment, the live biotherapeutic secrete synthetic therapeutic bacteriophages displaying one or more tumor antigen peptides. There are numerous known tumor antigens to date, e.g. tumor specific antigens, tumor-associated antigens (TAAs) and neoantigens, many of which are associated with certain tumors and cancer cells. These tumor antigens are typically small peptide antigens, associated with a certain cancer cell type, which are known to stimulate an immune response. By introducing such tumor antigens, e.g., tumor-specific antigens, TAA(s), and/or neoantigen(s) to the local tumor environment, an immune response can be raised against the particular cancer or tumor cell of interest known to be associated with that neoantigen. In some embodiments, the one or more tumor antigen peptide displayed by the synthetic therapeutic phage can be any known or later discovered tumor antigen associated with cancer cells. The one or more tumor antigen peptides can be selected from, but not limited to, Tables 26, 27, 28, 29, 30, 31, and 32 from PCT/US2017/013072 (incorporated herein by reference).

In an embodiment, the one or more peptides displayed by the synthetic therapeutic bacteriophages can be the peptide sequences of a receptor ligands, or fragments of receptor ligands, that activate cellular receptors that lead to the elimination of cancerous cells. The one or more peptide ligand can be any known or later discovered peptide ligand that activate cellular receptors that lead to the elimination of cancerous cells. In some embodiments, the one or more peptide ligand sequences are derived from, but not limited to, CD40L, CD80, CD86, ICOS ligand, CD112, CD155, CD137 ligand, and CD134 ligand.

In an embodiment, the one or more peptides displayed by the synthetic therapeutic bacteriophages can be lytic peptides that eliminate tumor cells. synthetic therapeutic bacteriophage.

In an embodiment, the therapeutic bacteriophages displays one or more enzymes that activate prodrugs. synthetic therapeutic bacteriophage Examples of enzymes that activate prodrugs for the treatment of cancers include, but are not limited to, cytosine deaminase, purine nucleoside phosphorylase, deoxycytidine kinase, thymidylate kinase, and uridine monophosphate kinase.

In an embodiment, the therapeutic bacteriophages displays one or more enzymes that deplete metabolites essential for tumor and cancer cells proliferation. Example of metabolites important for the tumor and cancer cell proliferation includes, but are not limited to, L-asparagine, L-glutamine, L-methionine, and kynurenine. The one or more enzymes depleting metabolites essential for tumor and cancer cells proliferation displayed by the synthetic therapeutic bacteriophages can be any known or later discovered enzymes depleting metabolites essential for tumor and cancer cells proliferation. Examples of enzymes depleting metabolites essential for tumor and cancer cells proliferation include, but not limited to, L-asparaginase, L-glutaminase, methioninase, and kynureninase.

In an embodiment, the synthetic therapeutic bacteriophages display a combination of one or more enzymes that activate prodrugs and one or more enzymes that deplete metabolites essential for tumor and cancer cells proliferation.

As a mean to potentiate the antitumoral effect of the synthetic bacteriophage, the bacterial host secreting the therapeutic bacteriophage can naturally, or after genetic engineering, execute additional therapeutic activities to stimulate the immune response. Many immune cells found in the tumor microenvironment express pattern recognition receptors (PRRs), which receptors play a key role in the immune response through the activation of pro-inflammatory signaling pathways, stimulation of phagocytic responses (macrophages, neutrophils and dendritic cells) or binding to micro-organisms as secreted proteins. PRRs recognize two classes of molecules: pathogen-associated molecular patterns (PAMPs), which are associated with microbial pathogens, and damage-associated molecular patterns (DAMPs), which are associated with cell components that are released during cell damage, death stress, or tissue injury. PAMPS are essential molecular structures required for pathogens survival, e.g., bacterial cell wall molecules (e.g. lipoprotein), bacterial DNA. PRRs are expressed by cells of the innate immune system but can also be expressed by other cells (both immune and non-immune cells). PRR are either localized on the cell surface to detect extracellular pathogens or within the endosomes and cellular matrix where they detect intracellular invading viruses. Examples of PRRs include Toll-like receptors (TLR1, TLR 2, TLR3, TLR4, TLR5, TLR6, TLR7, TLR8, TLR9, TLR10), C-type lectin receptors (Group I mannose receptors and group II asialoglycoprotein), nucleotide oligomerization (NOD)-like receptors (NODI and NOD2), retinoicacid-inducible gene I (RIG-I)-like receptors (RLR) (RIG-I, MDA5, and DDX3), collectins, pentraxins, ficolins, lipid transferases, peptidoglycan recognition proteins (PGRs) and the leucine-rich repeat receptor (LRR). Upon detection of a pathogen, PRRs activate inflammatory and immune responses mounted against the infectious pathogen. Recent evidence indicates that immune mechanisms activated by PAMPs and DAMPs play a role in activating immune responses against tumor cells as well (Hobohm & Grange, Crit Rev Immunol. 2008; 28(2):95-107, and Krysko D V et al., Cell Death and Disease (2013) 4, e631; here in incorporated as references). The bacterial host of the present disclosure can trigger an immune response through the presence of PAMPs and DAMPs, which are agonists for PRRs found on immune cells and tumor cells in the tumor microenvironment. Thus, in some embodiments, the bacterial host of the present disclosure trigger an immune response at the tumor site (FIG. 4). In these embodiments, the microorganism naturally expresses a PRR agonist, such as one or more PAMPs or DAMPs.

The bacterial host secreting the synthetic therapeutic bacteriophages can be engineered to secrete or produce one or more immunostimulatory enzymes in order to prevent the growth of tumor cells.

In an embodiment, the bacterial host secretes or produces one or more enzymes that deplete metabolites essential for tumor and cancer cells proliferation.

In an embodiment, the bacterial host secrete or produce 15-hydroxyprostaglandin dehydrogenase (15-PGDH), which converts Prostaglandin E2 (PGE2) into 15-keto-PGs. Prostaglandin E2 (PGE2) is overproduced in many tumors, where it aids in cancer progression. PGE2 is a pleiotropic molecule involved in numerous biological processes, including angiogenesis, apoptosis, inflammation, and immune suppression. Delivery of 15-PGDH locally to the tumor has been shown to resulted in significantly slowed tumor growth.

The bacterial host secreting the synthetic therapeutic bacteriophages can be engineered to secrete one or more immunostimulatory proteins in order to prevent the growth of tumor cells.

In an embodiment, the bacterial host secrete the Granulocyte-macrophage colony-stimulating factor (GM-CSF). GM-CSF is part of the immune/inflammatory cascade. GM-CSF activation of a small number of macrophages rapidly leads to an increase in their numbers. It has been shown that GM-CSF can be used as an immunostimulatory adjuvant to elicit antitumor immunity.

In an embodiment, the bacterial host secrete one or more cytokines that stimulate and/or induce the differentiation of T effector cells, e.g., CD4+ and/or CD8+. Cytokines that stimulate and/or induce the differentiation of T effector cells includes, but are not limited to, IL-2, IL-15, IL-12, IL-7, IL-21, IL-18, TNF, and interferon gamma (IFN-gamma). The one or more cytokines that stimulate and/or induce the differentiation of T effector cells secreted by the bacterial host can be any known or later discovered cytokines that stimulate and/or induce the differentiation of T effector cells.

In an embodiment, the bacterial host secrete tryptophan. The catabolism of tryptophan is a central pathway maintaining the immunosuppressive microenvironment in many types of cancers.

In an embodiment, the bacterial host secrete L-arginine. In human, the absence of arginine in the tumor microenvironment inhibits the progression of T lymphocytes through the cell cycle via induction of a G0-G1 arrest, and thus acts as immunosuppressor preventing the elimination of cancer cells. Therefore, in some embodiment the bacterial host can be engineered to comprise one or more gene sequences encoding one or more enzymes of the arginine pathway. Genes involved in the arginine pathway include, but are not limited to, argA, argB, argC, argD, argE, argF, argG, argH, argL, arg.J, carA, and carB. These genes may be organized, naturally or synthetically, into one or more operons. All of the genes encoding these enzymes are subject to repression by arginine via its interaction with ArgR to form a complex that binds to the regulatory region of each gene and inhibits transcription. In some embodiments, the genetically engineered bacteria of the present technology comprise one or more nucleic acid mutations that reduce or eliminate arginine-mediated repression of one or more of the operons that encode the enzymes responsible for converting glutamate to arginine and/or an intermediate by-product in the arginine biosynthesis pathway.

In some embodiments, the bacterial host is engineered to import adenosine to decrease the level of adenosine in the tumor microenvironment. Adenosine is a potent immunosuppressive molecule found in tumor microenvironment, therefore decreasing its level increases the immune response against tumor cells. The adenosine import mechanism can be derived from any known or later discovered E. coli nucleoside permeases. In an embodiment, the engineered bacterial host import adenosine via the E. coli Nucleoside Permease nupG or nupC.

In some embodiments, the present technology relates to the use of the live biotherapeutic described in the present disclosure and/or the synthetic therapeutic bacteriophages for, but not limited to, the treatment of cancer and/or treatment of tumors. A tumor may be malignant or benign. Types of cancer that may be treated using the present technology include, but are not limited to: adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, largyngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer, lymphoma (e.g., AIDS-related lymphoma, Burkitt lymphoma, cutaneous T cell lymphoma, Hodgkin lymphoma, Non-Hodgkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenstrom macrogloblulinemia, and Wilms tumor. In some embodiments, the symptom(s) associated thereof include, but are not limited to, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration.

In some embodiments, the methods of the present technology include administering an effective amount of at least one live biotherapeutics and/or at least one synthetic therapeutic bacteriophage described herein to a subject in need thereof. The live biotherapeutic and/or the synthetic therapeutic bacteriophage may be administered locally, e.g., intratumorally or peritumorally into a tissue or supplying vessel, intramuscularly, intraperitoneally, orally, topically, or by instillation in the bladder. The live biotherapeutic and/or the synthetic therapeutic bacteriophage may be administered systemically, e.g., intravenously or intra-arterially, by infusion or injection.

In some embodiments, the present technology relates to compositions such as pharmaceutical compositions, comprising at least one biotherapeutics and/or at least one synthetic therapeutic bacteriophage described herein and optionally one or more suitable pharmaceutical excipient, diluent or carrier. In certain embodiments, administering the at least one live biotherapeutics and/or at least one synthetic therapeutic bacteriophage described herein to a subject in need thereof or administering the composition of the present technology reduces cell proliferation, tumor growth, and/or tumor volume in a subject. In some embodiments, the methods of the present disclosure may reduce cell proliferation, tumor growth, and/or tumor volume by at least about 10%, 20%, 25%, 30%, 40%, 50%, 60%, 70%, 75%, 80%, 85%, 90%, 95%, or more as compared to levels in an untreated or control subject. In some embodiments, reduction is measured by comparing cell proliferation, tumor growth, and/or tumor volume in a subject before and after administration of the pharmaceutical composition. In some embodiments, the method of treating or ameliorating a cancer in a subject allows one or more symptoms of the cancer to improve by at least about 10%, 20%, 30%, 40%, 50%, 60%, 70%, 80%, 90%, 95%, or more. Before, during, and after the administration of the pharmaceutical composition, cancerous cells and/or biomarkers in a subject may be measured in a biological sample, such as blood, serum, plasma, urine, peritoneal fluid, and/or a biopsy from a tissue or organ. In some embodiments, the methods may include administration of the compositions of the present technology to reduce tumor volume in a subject to an undetectable size, or to less than about 1/a, 2%, 5%, 10%, 20%, 25%, 30%, 40%, 50%, 60%, 70%, 75%, 80%, or 90% of the subject's tumor volume prior to treatment. In other embodiments, the methods may include administration of the compositions of the present technology to reduce the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment.

In some embodiments, the compositions of the present technology may be administered alone or in combination with one or more additional therapeutic agents. Non-limiting examples of therapeutic agents include conventional therapies (e.g., radiotherapy, chemotherapy), immunotherapies (e.g. vaccines, dendritic cell vaccines, or other vaccines of other antigen presenting cells, checkpoint inhibitors, cytokine therapies, tumor infiltrating lymphocyte therapies, native or engineered TCR or CAR-T therapies, natural killer cell therapies, Fc-mediated ADCC therapies, therapies using bispecific soluble scFvs linking cytotoxic T cells to tumor cells, and soluble TCRs with effector functions), stem cell therapies, and targeted therapies with antibodies or chemical compounds (e.g., BRAF or vascular endothelial growth factor inhibitors), synthetic bacteriophages. In some embodiments, the genetically engineered bacteria are administered sequentially, simultaneously, or subsequently to dosing with one or more chemotherapeutic agents selected from, but not limited to, methotrexate, Trabectedin®, Belotecan®, Cisplatin®, Carboplatin®, Bevacizumab®, Pazopanib®, 5-Fluorouracil, Capecitabine® Irinotecan®, Gemcitabine (Gemzar), and Oxaliplatin®.

In some embodiments, the at least one live biotherapeutics is administered sequentially, simultaneously, or subsequently to dosing with one or more of the following checkpoint inhibitors or other antibodies known in the art or described herein. Nonlimiting examples include CTLA-4 antibodies (including but not limited to Ipilimumab and Tremelimumab (CP675206)), anti-4-1BB (CD 137, TNFRSF9) antibodies (including but not limited to PF-05082566, and Urelumab), anti CD134 (OX40) antibodies, including but not limited to Anti-OX40 antibody (Providence Health and Services), anti-PD1 antibodies (including but not limited to Nivolumab, Pidilizumab, Pembrolizumab (MK-3475/SCH900475, lambrolizumab, REGN2810, PD1 (Agenus)), anti-PD-L1 antibodies (including but not limited to Durvalumab (MEDI4736), Avelumab (MSB0010718C), and Atezolizumab (MPDL3280A, RG7446, R05541267)), andit-KIR antibodies (including but not limited to Lirilumab), LAG3 antibodies (including but not limited to BMS-986016), anti-CCR4 antibodies (including but not limited to Mogamulizumab), anti-CD27 antibodies (including but not limited to Varlilumab), anti-CXCR4 antibodies (including but not limited to Ulocuplumab).

In some embodiments, the at least one live biotherapeutics and/or one synthetic therapeutic bacteriophage is administered sequentially, simultaneously, or subsequently to dosing with one or more antibodies selected from an antiphosphatidyl serine antibody (including but not limited to Bavituxumab), TLR9 antibody (including, but not limited to, MGN1703 PD1 antibody (including, but not limited to, SHR-1210 (Incyte/Jiangsu Hengrui)), anti-OX40 antibody (including, but not limited to, OX40 (Agenus)), anti-Tim3 antibody (including, but not limited to, Anti-Tim3 (Agenus/INcyte)), anti-Lag3 antibody (including, but not limited to, Anti-Lag3 (Agenus/INcyte)), anti-B7H3 antibody (including, but not limited to, Enoblituzumab (MGA-271), anti-CT-011 (hBAT, hBAI1) as described in WO2009101611 (incorporated herein by reference), anti-PDL-2 antibody (including, but not limited to, AMP-224 (described in WO2010027827 and WO201 1066342; incorporated herein by reference), anti-CD40 antibody (including, but not limited to, CP-870, 893), anti-CD40 antibody (including, but not limited to, CP-870, 893). Pharmaceutical compositions comprising the live biotherapeutics and/or the synthetic therapeutic bacteriophage of the present technology may be used to treat, manage, ameliorate, and/or prevent cancer. Pharmaceutical compositions of the present technology comprising one or more live biotherapeutics alone or in combination with prophylactic agents, therapeutic agents, and/or pharmaceutically acceptable carriers are provided. In certain embodiments, the pharmaceutical composition comprises one live biotherapeutic engineered to comprise the genetic modifications described herein, e.g., one or more genes encoding one or more anti-cancer molecules. In alternate embodiments, the pharmaceutical composition comprises two or more live biotherapeutics that are each engineered to comprise the genetic modifications described herein, e.g., one or more genes encoding one or more anti-cancer molecules. In yet another embodiments, the pharmaceutical composition comprises the synthetic therapeutic bacteriophage that are each engineered to display the recombinant protein described herein, e.g., one or more anti-cancer molecule.

The pharmaceutical compositions of the present technology may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, PA). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The live biotherapeutics and/or the synthetic therapeutic bacteriophage may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, intratumoral, peritumor, immediate release, pulsatile-release, delayed-release, or sustained release). Suitable dosage amounts for the live biotherapeutics may range from about 104 to 10¹¹ bacteria. The composition may be administered once or more daily, weekly, monthly, or annually. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal.

The live biotherapeutics and/or the synthetic therapeutic bacteriophage may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the present live biotherapeutics may be formulated in a solution of sodium bicarbonate, e.g., 1 molar solution of sodium bicarbonate (to buffer an acidic cellular environment, such as the stomach, for example). the live biotherapeutics may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The live biotherapeutics and/or the synthetic therapeutic bacteriophage may be administered intravenously, e.g., by infusion or injection. Alternatively, the live biotherapeutics and/or the synthetic therapeutic bacteriophage may be administered intratumorally and/or peritumorally. In other embodiments, the live biotherapeutics and/or the synthetic therapeutic bacteriophage may be administered intra-arterially, intramuscularly, or intraperitoneally. In some embodiments, the live biotherapeutics colonize about 20/a, 30%, 40%, 50%, 60%, 70%, 80%, 90% or more of the tumor. In some embodiments, the live biotherapeutics and/or the synthetic therapeutic bacteriophage are co-administered with a PEGylated form of rHuPH20 (PEGPH20) or other agent in order to destroy the tumor septae in order to enhance penetration of the tumor capsule, collagen, and/or stroma. In some embodiments, the live biotherapeutics can produce an anti-cancer molecule as well as one or more enzymes that degrade fibrous tissue.

In some embodiments, the treatment regimen will include one or more intratumoral administrations. In some embodiments, a treatment regimen will include an initial dose, which followed by at least one subsequent dose. One or more doses can be administered sequentially in two or more cycles. For example, a first dose may be administered at day 1, and a second dose may be administered after 1, 2, 3, 4, 5, 6, days or 1, 2, 3, or 4 weeks or after a longer interval. Additional doses may be administered after 1, 2, 3, 4, 5, 6, days or after 1, 2, 3, or 4 weeks or longer intervals. In some embodiments, the first and subsequent administrations have the same dosage. In other embodiments, different doses are administered. In some embodiments, more than one dose is administered per day, for example, two, three or more doses can be administered per day.

The live biotherapeutics and/or synthetic therapeutic bacteriophage disclosed herein may be administered topically and formulated in the form of an ointment, cream, transdermal patch, lotion, gel, shampoo, spray, aerosol, solution, emulsion, or other form well known to one of skill in the art. See, e.g., “Remington's Pharmaceutical Sciences,” Mack Publishing Co., Easton, PA. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the live biotherapeutics of the present technology may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The live biotherapeutics and/or synthetic therapeutic bacteriophage disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., /actose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginatepolylysine-alginate (APA), alginate-polymethylene-co-guanidine-alginate (A-PMCG-A), hydroymethylacrylate-methyl methacrylate (HEMA-MMA), multilayered HEMA-MMAMAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the live biotherapeutics and/or synthetic therapeutic bacteriophage are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

In some embodiments, enteric coating materials may be used, in one or more coating layers (e.g., outer, inner and/o intermediate coating layers). Enteric coated polymers remain unionised at low pH, and therefore remain insoluble. But as the pH increases in the gastrointestinal tract, the acidic functional groups are capable of ionisation, and the polymer swells or becomes soluble in the intestinal fluid.

The uses and methods defined herein comprise administering to a subject a therapeutically effective amount of live biotherapeutic or of the synthetic bacteriophage as defined herein to achieve the effects discussed here. As used herein, the expression “effective amount” or “therapeutically effective amount” refers to the amount of live biotherapeutic or of the synthetic bacteriophage as defined herein which is effective for producing some desired therapeutic effect as defined herein at a reasonable benefit/risk ratio applicable to any medical treatment. Therapeutically effective dosage of any specific peptide of the present disclosure will vary from subject to subject, and patient to patient, and will depend, among other things, upon the effect or result to be achieved, the condition of the patient and the route of delivery. The expressions “therapeutically acceptable”, “therapeutically suitable”, “pharmaceutically acceptable” and “pharmaceutically suitable” are used interchangeably herein and refer to a peptide, a compound, or a composition that is suitable for administration to a subject to achieve the effects described herein, such as the treatment defined herein, without unduly deleterious side effects in light of the severity of the disease and necessity of the treatment.

In another embodiment, the pharmaceutical composition comprising the live biotherapeutics of the present technology may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the live biotherapeutics of the present technology are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the live biotherapeutics of the present technology are well known in the art. For example, see U.S. 2015/0359894 and US 2015/0238545, the entire contents of each of which are expressly incorporated herein by reference. In yet another embodiment, the pharmaceutical composition of the present technology is injected into, sprayed onto, or sprinkled onto a food product, such as bread, yogurt, or cheese.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05/6, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the live biotherapeutics and/or synthetic therapeutic bacteriophage and composition thereof is formulated for intravenous administration, intratumor administration, or peritumor administration. The live biotherapeutics and/or synthetic therapeutic bacteriophage may be formulated as depot preparations. Such long acting formulations may be administered by implantation or by injection. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In another embodiment, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463; incorporated herein by reference). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The live biotherapeutics and/or synthetic therapeutic bacteriophage of the present technology may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

The examples below are given to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1: Engineering of the Live Biotherapeutic Secreting Synthetic Bacteriophages for Display of Therapeutic Proteins

All strains and plasmids used in this Example are described in Table 1. Cells were typically grown in Luria broth Miller (LB) or on Luria broth agar Miller medium supplemented, when needed, with antibiotics at the following concentrations: ampicillin (Ap) 100 μg/mL, chloramphenicol (Cm) 34 μg/mL, kanamycin (Km) 50 μg/mL, nalidixic acid (Nx) 4 μg/mL, spectinomycin (Sp) 100 μg/mL, streptomycin (Sm) 50 μg/mL, sulfamethoxazole (Su) 160 μg/mL, tetracycline (Tc) 15 μg/mL, and trimethoprim (Tm) 32 μg/mL. All cultures were routinely grown at 37° C. Cells with thermosensitive plasmids (pTAT00X, pTAT001) were grown at 30° C. No bacterial cultures over 18 hours of age were used in the experiments.

TABLE 1
List of strains and plkasmids used for the study

| Strain or | | |
|---|---|---|
| plasmid | Relevant phenotype or genotype | Source/Reference |
| E. coli | | |
| MG1655 | F- lambda- ilvG- rfb-50 rph-1, OR: H48: K- | CGSC#: 7636 |
| EC100Dpir+ | F- mcrA Δ(mrr-hsdRMS-mcrBC) ϕ80dlacZΔM15 ΔlacX74 | #ECP09500 |
| | recA1 endA1 araD139 Δ(ara, leu)7697 galU galK α- rpsL | (Lucigen) |
| | nupG pir+ (DHFR) | |
| ER2738 | F′proA⁺B⁺ lacI^{q} Δ(lacZ)M15 zzf::Tn10(Tet^{R})/fhuA2 glnV | E4104 (NEB) |
| | Δ(lac-proAB) thi-1 Δ(hsdS-mcrB)5 | |
| KN01 | Sm^{R}Sp^{R} Nissle 1917 | Neil et al. 2020 |
| MM294 | glnV44(AS) rfbC1 endA1 spoT1 thi-1 hsdR17 creC510 | DSM 5208 |
| Plasmid | | |
| M13K07 | M13Δori_{M13}::oriV_{p15A}-aph-III | N0315S (NEB) |
| M13mp18-Kan | M13mp18ΔlacZ::aph-III | Example I |
| pKN23 | oriV_{pSC101ts}, gRNA, cas9, aph-III | TATUM plasmid |
| | | Genbank: |
| | | MK756312.1 |
| pSB1C3 | oriV_{pMB1}, cat, Biobrick | IGEM |
| pTAT00X | oriV_{pSC101ts}, Tn7 insertion machinery, araC, bla | TATUM plasmids |
| pTAT001 | pTAT00X::M13K07ΔgpIII-ori_{M13}-oriV_{p15A}-aphIII | Example 1 |
| pTAT002 | oriV_{pMB1}, aad7, ori_{M13}, P5-BCD1-N-gpIII-HA-6His-gpIII-C | Example 1 |
| pTAT003 | oriV_{pMB1}, aad7, ori_{M13}, P5mut-BCD1-pelB-nbCD47_A4- | Example 1 |
| | HA-6His-gpIII-C | |
| pTAT004 | M13K07ΔgpIII | Example 1 |
| pTAT010-P5 | oriV_{R6K}, cat, P5-BCD1-sfGFP | Example 1 |
| pTAT010-P5mut | oriV_{R6K}, cat, P5mut-BCD1-sfGFP | Example 1 |
| pTAT012 | oriV_{pMB1}, aad7, ori_{M13} | Example 1 |
| pTAT013 | GAT01, ori_{M13}, aad7, GAT04, oriV_{pMB1}, GAT07, P5-BCD1- | Example 1 |
| | sfGFP | |
| pTAT014 | GAT01, bla, GAT04, oriV_{pSC101}, GAT07, P5-BCD1-sfGFP | Example 1 |
| pTAT017 | GAT01, bla, GAT04, oriV_{pSC101}, GAT07, P5-BCD1-gpIX | Example 1 |
| pTAT019 | GAT01, ori_{M13}, aad7, GAT04, oriV_{pMB1}, GAT07, P5mut- | Example 1 |
| | BCD1-pelB-nbCTLA-4-HA-6His-gpIII-C | |
| pTAT020 | GAT01, ori_{M13}, aad7, GAT04, oriV_{pMB1}, GAT07, P5mut- | Example 1 |
| | BCD1- pelB-nbPDL1- HA-6His-gpIII-C | |
| pTAT020-GTG | GAT01, ori_{M13}, aad7, GAT04, oriV_{pMB1}, GAT07, P5mut- | Example 1 |
| | BCD1-GTG-pelB-nbPDL1- HA-6His-gpIII-C | |
| pTAT022 | GAT01, ori_{M13}, aad7, GAT04, oriV_{pMB1}, GAT07, P5mut- | Example 1 |
| | BCD1-pelB-codA-HA-6His-tags-gpIII-C | |
| PTAT025 | M13K07ΔgpIIIΔgpIX | Example 1 |
| pTAT027 | M13K07ΔgpIIIΔgpVIII::OVA-gpVIII | Example 1 |
| pTAT028 | GAT01, bla, GAT04, oriV_{pSC101}, GAT07, P14-revtetR, | Example 1 |
| | P_{tetO}-BCD1-pelB-nbCD47_A4-gpIX | |
| pTAT030 | GAT01, ori_{M13}, aad7, GAT04, oriV_{pMB1}, GAT07, P5mut- | Example 1 |
| | BCD1-pelB-anticalinCTLA-4-HA-6His-gpIII-C | |
| PTAT032 | M13mp18-KanΔgpIII::GTG-pelB-nbPDL1-gpIII-C | Example 1 |
| pTAT032ΔgpIX | M13mp18-KanΔgpIII::GTG-pelB-nbPDL1-gpIII-C ΔgpIX | Example 1 |
| PTAT033 | M13mp18-KanΔgpIII::N-gpIII-nbPDL1-gpIII-C | Example 1 |
| pTAT034 | lacI, bla, oriV_{ColE1}, nbPDL1 | Example 1 |
| PTAT035 | GAT01, bla, GAT04, oriV_{pSC101}, GAT07, P5mut-BCD1- | Example I |
| | GTG-pelB-anticalinCTLA4-FLAG-gpIX | |
| pTRC-HisB | lacI, bla, oriV_{ColE1} | TATUM bioscience |

DNA manipulations. A detailed list of oligonucleotide sequences used in this Example is found in Table 2. Plasmids were prepared using EZ10-Spin Column Plasmid Miniprep kit (BIOBASIC #BS614) or QIAGEN Plasmid Maxi Kit (QIAGEN) according to the manufacturer's instructions. PCR amplifications were performed using TransStart FastPFU fly DNA polymerase (Civic Bioscience) for DNA parts amplification and screening. Digestion with restriction enzymes used products from NEB and were incubated for 1 hour at 37° C. following manufacturer's recommendations. Plasmids were assembled by Gibson assembly using the NEBuilder HiFi DNA Assembly Master Mix (NEB) following manufacturer's protocol.

TABLE 2
Oligonucleotide sequences

| | Length | | | |
|---|---|---|---|---|
| Name^{a} | (bp) | Template | Description | Construct |
| SEQ ID NO.: 1 | 2964 | pTAT00X | Amplify pTAT001 | pTAT001 |
| SEQ ID NO.: 2 | | | backbone | |
| SEQ ID NO.: 3 | 3136 | pTAT00X | Amplify pTAT001 | |
| SEQ ID NO.: 4 | | | backbone | |
| SEQ ID NO.: 5 | 3127 | pTAT00X | Amplify pTAT001 | |
| SEQ ID NO.: 6 | | | backbone | |
| SEQ ID NO.: 7 | 3637 | pTAT00X | Amplify pTAT001 | |
| SEQ ID NO.: 8 | | | backbone | |
| SEQ ID NO.: 9 | 2200 | M13K07 | Amplify 1st part of | |
| SEQ ID NO.: 10 | | | hyperphage | |
| SEQ ID NO.: 11 | 3034 | M13K07 | Amplify 2nd part of | |
| SEQ ID NO.: 12 | | | hyperphage | |
| SEQ ID NO.: 13 | 420 | M13K07 | Amplify 1st part of ori_{M13} | pTAT003 |
| SEQ ID NO.: 14 | | | | |
| SEQ ID NO.: 15 | 178 | M13K07 | Amplify 2nd part of ori_{M13} | |
| SEQ ID NO.: 16 | | | | |
| SEQ ID NO.: 17 | 1200 | N16Spec | Amplify aad7 | |
| SEQ ID NO.: 18 | | | | |
| SEQ ID NO.: 19 | 885 | pSB1C3 | Amplify oriV_{pMB1} | |
| SEQ ID NO.: 20 | | | | |
| SEQ ID NO.: 21 | 830 | gBlock_TKN1 | Amplify nbCD47_A4 | |
| SEQ ID NO.: 22 | | | | |
| SEQ ID NO.: 23 | 601 | M13K07 | Amplify gpIII C-terminal | |
| SEQ ID NO.: 24 | | | domains | |
| SEQ ID NO.: 25 | 3200 | pTAT003 | Amplify backbone | pTAT002 |
| SEQ ID NO.: 26 | | | | |
| SEQ ID NO.: 27 | 741 | M13K07 | Amplify the N-terminal half | |
| SEQ ID NO.: 28 | | | of gpIII | |
| SEQ ID NO.: 29 | 2200 | M13K07 | Amplify parts of M13K07 | pTAT004 |
| SEQ ID NO.: 10 | | | except for gpIII | |
| SEQ ID NO.: 11 | 3034 | M13K07 | | |
| SEQ ID NO.: 30 | | | | |
| SEQ ID NO.: 31 | 2512 | M13K07 | | |
| SEQ ID NO.: 32 | | | | |
| SEQ ID NO.: 33 | 176 | ori_{M13} | qPCR-phage pulldown | qPCR |
| SEQ ID NO.: 34 | | | | |
| SEQ ID NO.: 35 | 193 | gpIII | qPCR-phage pulldown | |
| SEQ ID NO.: 36 | | | | |
| SEQ ID NO.: 37 | 135 | aad7 | qPCR-phage pulldown | |
| SEQ ID NO.: 38 | | | | |
| SEQ ID NO.: 39 | 2710 | PSW23T-PL-sfGFP | GFP promotor test | pTAT010-P5 and |
| SEQ ID NO.: 40 | | | backbone | pTAT010-P5mut |
| SEQ ID NO.: 41 | 350 | gBlock_TKN1 or | P5-BCD1 or P5mut-BCD1 | |
| SEQ ID NO.: 42 | | pTAT003 | | |
| SEQ ID NO.: 43 | 2380 | pTAT002 | Amplify 1st part of the | pTAT012 |
| SEQ ID NO.: 16 | | | backbone | |
| SEQ ID NO.: 44 | 2380 | pTAT002 | Amplify 2nd part of the | |
| SEQ ID NO.: 17 | | | backbone | |
| SEQ ID NO.: 45 | 1706 | pTAT002 | ori_{M13} + aad7 + GAT04 | pTAT013 |
| SEQ ID NO.: 46 | | | | |
| SEQ ID NO.: 47 | 869 | pTAT002 | oriV_{pMB1} + GAT04 + | |
| SEQ ID NO.: 48 | | | GAT07 | |
| SEQ ID NO.: 49 | 1111 | pTAT010-P5 | P5-BCD1-sfGFP + | |
| SEQ ID NO.: 50 | | | GAT01/07 | |
| SEQ ID NO.: 51 | 1226 | pUC19 | Amplify bla + GAT04 | pTAT014 |
| SEQ ID NO.: 52 | | | | |
| SEQ ID NO.: 53 | 1010 | pKN23 | Amplify 1st half of | |
| SEQ ID NO.: 54 | | | oriV_{pSc101} + GAT04 | |
| SEQ ID NO.: 55 | 1310 | pKN23 | Amplify 2nd half of | |
| SEQ ID NO.: 56 | | | oriV_{pSC101} + GAT04 | |
| SEQ ID NO.: 49 | 1111 | pTAT010-P5 | P5-BCD1-sfGFP + | |
| SEQ ID NO.: 50 | | | GAT01/07 | |
| SEQ ID NO.: 57 | 3001 | pTAT014 | Amplify the backbone | pTAT017 |
| SEQ ID NO.: 58 | | | | |
| SEQ ID NO.: 59 | 164 | M13K07 | Amplify p9 + GAT01 | |
| SEQ ID NO.: 60 | | | | |
| SEQ ID NO.: 57 | 2539 | pTAT013 | Amplify the backbone | pTAT019, |
| SEQ ID NO.: 48 | | | | pTAT020, |
| SEQ ID NO.: 49 | 298 | pTAT010-P5mut | Amplify P5mut-BCD1 | pTAT022, |
| SEQ ID NO.: 58 | | | | pTAT030. |
| SEQ ID NO.: 61 | 609 | M13K07 | Amplify gpIII C-terminal + | Assemble with |
| SEQ ID NO.: 62 | | | GAT01 | gBlock encoding |
| | | | | displayed protein |
| SEQ ID NO.: 57 | 2539 | pTAT013 | Amplify the backbone | pTAT020-GTG |
| SEQ ID NO.: 48 | | | | |
| SEQ ID NO.: 49 | 298 | pTAT010-P5mut | Amplify P5mut-BCD1 | |
| SEQ ID NO.: 58 | | | | |
| SEQ ID NO.: 61 | 609 | M13K07 | Amplify gpIII C-terminal + | |
| SEQ ID NO.: 62 | | | GAT01 | |
| SEQ ID NO.: 63 | 528 | gBlock_TAT04 | Modify start codon to GTG | |
| SEQ ID NO.: 64 | | | | |
| SEQ ID NO.: 29 | 1806 | pTAT004 | Amplify pTAT025 part 1 | pTAT025 |
| SEQ ID NO.: 65 | | | | |
| SEQ ID NO.: 66 | 3460 | pTAT004 | Amplify pTAT025 part 2 | |
| SEQ ID NO.: 30 | | | | |
| SEQ ID NO.: 31 | 2512 | M13K07 | Amplify pTAT025 part 3 | |
| SEQ ID NO.: 32 | | | | |
| SEQ ID NO.: 67 | 1806 | pTAT004 | Amplify pTAT027 part 1 | pTAT027 |
| SEQ ID NO.: 29 | | | | |
| SEQ ID NO.: 68 | 3219 | pTAT004 | Amplify pTAT027 part 2 | |
| SEQ ID NO.: 30 | | | | |
| SEQ ID NO.: 31 | 2512 | pTAT004 | Amplify pTAT027 part 3 | |
| SEQ ID NO.: 32 | | | | |
| SEQ ID NO.: 69 | 3600 | pTAT017 | Amplify backbone + gpIX | pTAT028 |
| SEQ ID NO.: 56 | | | | |
| SEQ ID NO.: 49 | 1230 | gBlock_TAT09 | Amplify revtetR + P_{tetO}- | |
| SEQ ID NO.: 70 | | | BCD1 | |
| SEQ ID NO.: 71 | 533 | gBlock_TKN1 | pTAT028-CD47nb | |
| SEQ ID NO.: 72 | | | | |
| SEQ ID NO.: 73 | 1258 | M13K07 | Amplify aph-III (Kan) | M13mp18-Kan |
| SEQ ID NO.: 74 | | | | |
| SEQ ID NO.: 75 | 3550 | M13mp18 | Amplify M13mp18-Kan | |
| SEQ ID NO.: 36 | | | part 1 | |
| SEQ ID NO.: 35 | 3731 | M13mp18 | Amplify M13mp18-Kan | |
| SEQ ID NO.: 76 | | | part 2 | |
| SEQ ID NO.: 77 | 529 | gBlock_TAT04 | Amplify nbPDL1 | pTAT032 |
| SEQ ID NO.: 64 | | | | |
| SEQ ID NO.: 78 | 1838 | M13mp18-kan | Amplfy pTAT032 part 1 | |
| SEQ ID NO.: 79 | | | | |
| SEQ ID NO.: 80 | 2325 | M13mp18-kan | Amplfy pTAT032 part 2 | |
| SEQ ID NO.: 81 | | | | |
| SEQ ID NO.: 23 | 3181 | M13mp18-kan | Amplfy pTAT032 part 3 | |
| SEQ ID NO.: 82 | | | | |
| SEQ ID NO.: 77 | 529 | gBlock_TAT04 | Amplify nbPDL1 | pTAT033 |
| SEQ ID NO.: 64 | | | | |
| SEQ ID NO.: 78 | 2579 | M13mp18-kan | Amplfy pTAT033 part 1 | |
| SEQ ID NO.: 83 | | | | |
| SEQ ID NO.: 80 | 2325 | M13mp18-kan | Amplfy pTAT033 part 2 | |
| SEQ ID NO.: 81 | | | | |
| SEQ ID NO.: 23 | 3181 | M13mp18-kan | Amplfy pTAT033 part 3 | |
| SEQ ID NO.: 82 | | | | |
| SEQ ID NO.: 84 | 503 | gBlock-TAT04 | Amplify nbPDL1 | pTAT034 |
| SEQ ID NO.: 85 | | | | |
| SEQ ID NO.: 86 | 2104 | pTrc-HisB | Amplify backbone part 1 | |
| SEQ ID NO.: 87 | | | | |
| SEQ ID NO.: 88 | 2345 | pTrc-HisB | Amplify backbone part 2 | |
| SEQ ID NO.: 89 | | | | |
| SEQ ID NO.: 49 | 678 | gBlock_TAT10 | Amplify anticalin-CLTA-4 | pTAT035 |
| SEQ ID NO.: 58 | | | | |
| SEQ ID NO.: 69 | 3790 | pTAT017 | Amplify backbone | |
| SEQ ID NO.: 56 | | | | |
| SEQ ID NO.: 90 | 300 | pTAT010-P5mut | Amplify P5mut | |
| SEQ ID NO.: 91 | | | | |
| SEQ ID NO.: 92 | Varies | Any of pTAT02-03, 19- | Amplify any displayed | Sanger |
| SEQ ID NO.: 93 | | 22, 28, 30 | protein | sequencing |
| SEQ ID NO.: 15 | 3375 | pTAT032 | Amplify first half of | pTAT032ΔgpIX |
| SEQ ID NO.: 65 | | | pTAT032 | |
| SEQ ID NO.: 66 | 4398 | pTAT032 | Amplify second half of | |
| SEQ ID NO.: 30 | | | pTAT032 | |

DNA purification. Purification of DNA was performed between each step of plasmid assembly to avoid buffer incompatibility or to stop enzymatic reactions. PCR reactions were generally purified by Solid Phase Reversible Immobilization (SPRI) using Agencourt Ampure XP DNA binding beads (Beckman Coulter) according to the manufacturer's guidelines or recovered and purified from agarose gel using Zymoclean Gel DNA Recovery Kit (Zymo Research). When DNA samples were digested with restriction enzymes, DNA was purified using Monarch® PCR & DNA Cleanup Kit (NEB) following manufacturer's recommendation for cell suspension DNA purification protocol. After purification, DNA concentration and purity were routinely assessed using a Nanodrop spectrophotometer when necessary.

DNA transformation into E. coli by electroporation. Routine plasmid transformations were performed by electroporation. Electrocompetent E. coli strains were prepared from 20 mL of LB broth. Cultures reaching exponential growth phase of 0.6 optical density at 600 nanometers (OD_{600 nm}) were then washed three times in sterile 10% of glycerol solution. Cells were then resuspended in 200 μL of water and distributed in 50 μL aliquots. The DNA was then added to the electrocompetent cells and the mixture was transferred in a 1 mm electroporation cuvette. Cells were electroporated using a pulse of 1.8 kV, 25 μF and 200Ω for 5 ms. Cells were then resuspended in 1 mL of non-selective LB medium and recovered for 1 hour at 37° C., or 30° C. for thermosensitive plasmid, before plating on selective media.

DNA transformation into E. coli by heat-shock. Heat-shock transformation was mostly used to clone Gibson assembly products. Chemically competent cells were prepared according to the rubidium chloride protocol as described previously (Green et al., 2013). Chemically competent cells were flash-frozen and conserved at −80° C. before use. Gibson assembly products were directly transformed into EC100Dpir+ or MM294 chemically competent cells at a 1/10 volume ratio. Routinely, up to 10 μL of DNA was added to 100 μL competent cells before transformation by a 45 seconds heat shock at 42° C. Cells were then resuspended in 1 mL of non-selective LB medium and let to recover for 1 hour at 37° C., or 30° C. for thermosensitive plasmid, before plating on selective media.

Insertion of pTAT00) in Escherichia coli genome as a biocontainment measure. The modified EcN::TAT001 strain is obtained by Tn7 insertion of the antibiotic resistance cassettes based on previously described procedures (McKenzie et al., 2006). Integration is verified by PCR using corresponding primers as described in Table 2. Loss of ampicillin resistance is confirmed to verify plasmid elimination. More specifically, the pTAT00X vector is purified from E. coli DH5-Alpha+ and digested with SmaI+XhoI. The inserts are amplified by PCR using their corresponding primers (Table 2) and inserted by Gibson assembly between attL_{Tn7} and attR_{Tn7} sites of the digested pTAT00X plasmid. The Gibson assembly products are then transformed in electrocompetent E. coli EC100Dpir+ strain. The resulting plasmids are analyzed using restriction enzymes, and positive clones are transformed into E. coli EC100ΔdapA+pTA-MOB. Plasmids are mobilized from E. coli EC100ΔdapA+pTA-MOB to MG1655 by conjugation. To mediate cassette insertion into the terminator of glmS, MG1655 is first cultivated at 30° C. in LB with 1% arabinose until 0.6 OD_{600 nm}. Cells are next heat-shocked at 42° C. for 1 hour and incubated at 37° C. overnight to allow for plasmid clearance. An aliquot of the bacterial culture is then streaked onto a LB agar plate. ≥20 colonies are analyzed, and colonies that only grow in the absence of ampicillin, but comprise the insert's selection markers, are then investigated by PCR using the appropriate primers listed in Table 2.

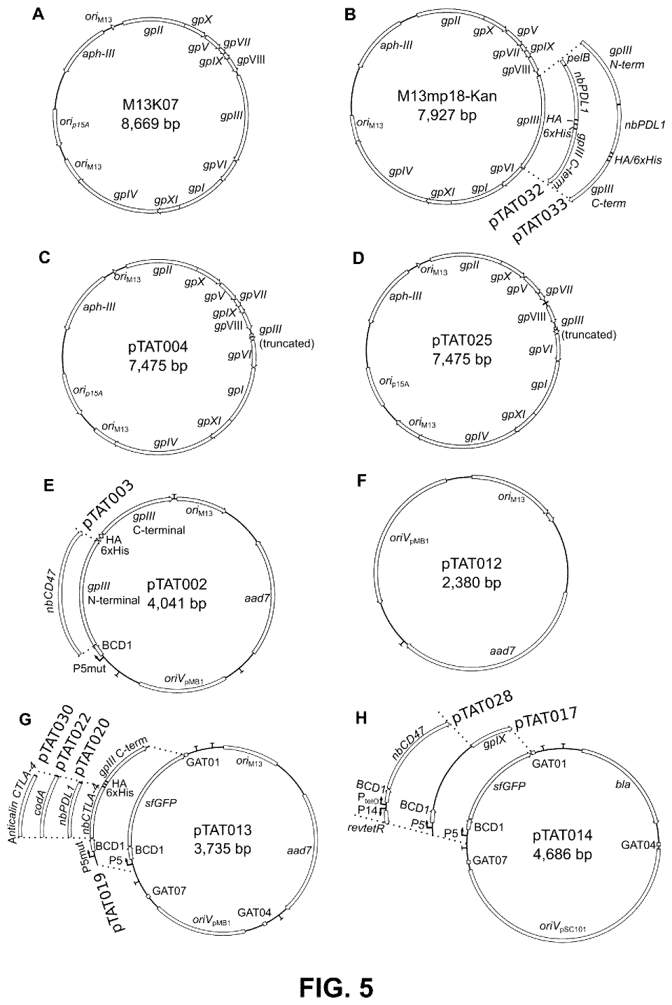

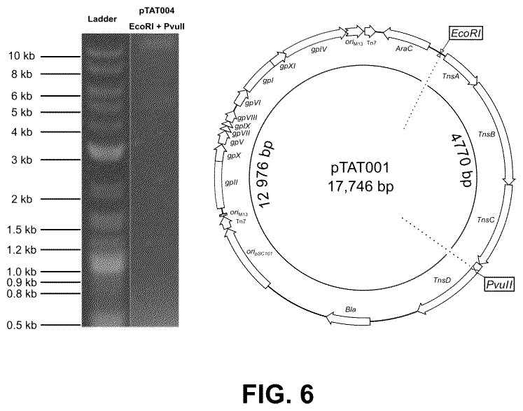

synthetic bacteriophage A live biotherapeutic secreting a synthetic bacteriophage for the display of therapeutic proteins was designed. A general description of the architecture of the live biotherapeutic is shown in FIG. 1 and a summary of the diverse constructs shown in FIGS. 5 and 6.

This example shows various iteration of the synthetic therapeutic bacteriophage secretion system and how those iterations can be exploited to display therapeutic proteins on various sites of the resulting bacteriophage particles. In a first form of the synthetic therapeutic bacteriophage secretion system, the system can be divided in a set of two vectors, the synthetic bacteriophage backbone vector (e.g. pTAT002 (FIG. 5E), pTAT003 (FIG. 5E), pTAT012 (FIG. 5F), pTAT013 (FIG. 5G), pTAT014 (FIG. 5H), pTAT019 (FIG. 5G), pTAT20 (FIG. 5G), pTAT022 (FIG. 5G), or pTAT030 (FIG. 5G)) and the synthetic bacteriophage machinery vector (e.g. M13K07 (FIG. 5A), pTAT004 (FIG. 5C), or pTAT025 (FIG. 5D)). In some cases, the synthetic therapeutic bacteriophage secretion system can be comprised in a single vector (e.g. M13mp18-Kan (FIG. 5B), pTAT032 (FIG. 5B), or pTAT033 (FIG. 5B)) or can be divided in three or more genetic constructs as in the system composed of pTAT025 (FIG. 5D), pTAT002 (FIG. 5E) and pTAT017 (FIG. 5H) or pTAT028 (FIG. 5H). To improve biocontainment of the synthetic therapeutic bacteriophage secretion system, some of its genes can be integrated in the chromosome of the host cell (as illustrated with pTAT001 see FIG. 6) as whole or in several parts, but still requires an extrachromomal DNA element comprising oriM13 (e.g. pTAT012) to act as a synthetic bacteriophage backbone vector.

All of the genes needed to assemble the bacteriophage are comprised in a single genetic element, much like the natural genome of M13. This conformation has the advantage to allow for the therapeutic fusion protein to benefit from the same levels of expression as the natural protein, which was optimized through milenia of evolution. The first step to obtain a single vector system was to modify M13mp18 to make it easily selectable. To do so, the primers listed in Table 2 were used to amplify M13mp18 and the aph-III gene from M13K07, which was designed to be inserted in the lacZ gene comprised in M13mp18. The fragments were next assembled by Gibson which resulted in the generation of M13mp18-Kan. Next, the therapeutic protein needed to be expressed from the same backbone. To achieve this, we amplified nbPDL1 (anti-PD-L1 nanobody) from a gBlock and all of the M13mp18-Kan backbone except for the N-terminal portion of gpIII, which was replaced by nbPDL1 in pTAT032 (FIG. 5). Another variant of the system (pTAT033) was designed to keep the gpIII gene in its entirety, but the nbPDL1 gene was inserted in the middle of pIII between the two domains that composed this coating protein (FIG. 5). Both systems were successfully assembled and produce functional synthetic therapeutic bacteriophages and are further described in Example 3. This illustrate that therapeutic protein fusion can be cloned directly in the synthetic bacteriophage machinery and produce synthetic therapeutic bacteriophages. Fusion with other capsid protein, such as gpVIII and gpLX are also be possible. This is further illustrated with pTAT027 in the next section.

To generate the synthetic bacteriophage machinery vector pTAT004, M13K07 was amplified in its entirety using appropriate primers presented in Table 2, except for the gpIII gene, which codes for pIII. The homology tails of the primers used for amplifying M13K07 were carefully designed to remove gpIII from the final construction. PCR products were next purified by SPRI and assembled by Gibson's method, generating pTAT004. The assembly was transformed into MM294 chemically competent cells and plasmid integrity was verified by digestion using NdeI. The pTAT004 synthetic bacteriophage machinery cannot produce fully functional bacteriophage particles on its own, as it does not possess a copy of the gpIII gene, and hence, does not produce pIII. In order to produce bacteriophage particles, gpIII must be provided in trans by the synthetic bacteriophage backbone vector. An another synthetic bacteriophage machinery was next derived from pTAT004. To demonstrate our ability to display proteins and peptides on pVIII, an epitope from the chicken ovalbumine gene was clone at the N-terminal of the gpVIII gene on pTAT004, generating pTAT027. This plasmid was assembled using primers to amplify the pTAT004 plasmid that introduced several mutations in the gpLX-gpVIII gene junctions. First, the start codon of gpVIII overlaps with the end of gpIX, so it was mutated and was re-introduced after the end of gpXI to allow further cloning. Then, the ovalbumine epitope was encoded in the primer tails and introduced right after the start codon of the gpVIII gene. This construct needs a external source of pIII for bacteriophages to be correctly assembled, but will display the OVA peptide on pVIII. The pTAT002, pTAT003, pTAT012, pTAT019, pTAT020, pTAT022, and pTAT030 synthetic bacteriophage backbone vectors were next designed. All synthetic bacteriophage backbone vectors comprise the ori_{pMB1} for high copy plasmid replication (maximising DNA material for encapsidation), ori_{M13} for ssDNA rolling circle replication and recognition of the synthetic bacteriophage backbone vector by the phage encapsidation machinery, a selective marker (here spectinomycin resistance), and a constitutively expressed pIII C-terminal fragment linked via one HA-His dual tag to either the N-terminal fragment of pIII (pTAT002, control with no therapeutic protein), or a checkpoint inhibitor binding protein (pTAT003, anti-CD47 nanobody; pTAT019, anti-CTLA-4 nanobody; pTAT020, anti-PD-L1 nanobody; pTAT22 and pTAT030, anti-CTLA-4 anticalin), or a therapeutic enzyme (pTAT022, cytosine deaminase (CD)) (see Table 3 for therapeutic protein sequences).

TABLE 3
Checkpoint inhibitor sequence list

| Name | SEQ ID NO: |
|---|---|
| Nanobody anti-CD47 | 94 |
| Nanobody anti-CTLA-4 | 95 |
| Nanobody anti-PD-L1 | 96 |
| Anticalin anti-CTLA4 | 97 |
| Cytosine Deaminase | 99 |

In this example, anti-CD47, anti-CTLA-4, and anti-PD-L1 binding proteins were selected as therapeutic agents because these are well characterized checkpoint inhibitors that binds to immune checkpoints expressed by cancerous cells (Vaddepally et al., Cancers (Basel) 2020 March; 12(3): 738; incorporated here by reference), while the cytosine deaminase is an enzyme that converts the 5-FC precursor into the 5-FU, a chemiotherapeutic agent commonly used to treat cancers (Nyati M. K. et al., Gene Therapy 2002; 9: 844-849; incorporated here by reference). The first synthetic bacteriophage backbone vector assembled was pTAT003. To build pTAT03, ori_{PMB1} was amplified by PCR from pSB1C3, ori_{M13} and the pIII N-terminal and C-terminal parts from M13K07, aad7 (spectinomycin resistance) from E. coli KN01 and gBlock comprising the anti-CD47 nanobody with a peptide linker and a constitutive promoter. The PCR products were next assembled by Gibson and transformed into chemically competent MM294 cells (FIG. 5). To assemble pTAT002, the backbone was amplified from pTAT003 and the missing N-terminal region of gpIII was amplified from M13K07. The two DNA parts were next assembled by Gibson's method. Integrity of the plasmid was next verified by digestion using ApaLI and NdeI. Plasmid pTAT012 was next generated using primers listed in Table 2 and assembled by Gibson assembly. The pTAT012 vector comprises only the ori_{M13}, oriV_{pMB1} and the aad7 resistance gene. It thus consists of a vector that complements M13K07, providing only a backbone for bacteriophage assembly and illustrate one biocomprisement strategy. Following sanger sequencing of pTAT002 and pTAT003, a mutation in the third position (G>T) of the P5 promoter was found in both pTAT003 and pTAT002. The resulting promoter, termed P5mut allow lower levels of expression of the upstream gene, as measured with the pTAT010-P5 and pTAT010-P5mut constructs using GFP (data not shown). In order to streamline construction assembly, the pTAT002 backbone was modified and a sfGFP gene was cloned to be expressed by the P5 promoter instead of gpIII. This backbone was assembled similarly to pTAT003, but the primer used allowed the insertion of an additional terminator after the gene expressed by P5, and the insertion of Gibson assembly tags (GAT) that allowed to delimit the different parts of the vector. The resulting vector, termed pTAT013, was then used as a template to amplify the backbone of the subsequent constructs for display of therapeutic proteins on pill. As such, for the construction of pTAT019, pTAT020, pTAT022, and pTAT030, the backbone of the plasmid was amplified from pTAT013, the P5mut promoter from pTAT010-P5mut, and the C-terminal part of gpIII from M13K07. Then, these DNA parts were assembled by gibson with different gBlocks encoding the therapeutic protein to display. As such, a gBlock encoding anti-CTLA-4 nanobody was used for pTAT019, an anti-PD-L1 nanobody was used for pTAT020, the cytosine deaminase codA was used for pTAT022, and an anti-CTLA-4 anticalin was used for pTAT030. All plasmids were next sent to Illumina or Sanger sequencing after assembly, no deleterious mutations were detected. With these results, the synthetic bacteriophage secretion system with a display of the therapeutic protein on pIII was ready for efficiency tests and improvement rounds. The synthetic therapeutic bacteriophage secretion system can be divided into three or more DNA molecules and remain functional as long a suffiscient proteins of each bacteriophage gene are produced. To illustrate the plasticity of the bacteriophage genome, we aimed to split the bacteriophage machinery into three different plasmids. As a first step, we needed to delete gpIII and an additional gene from M13K07. We selected gpLX, another capsid gene involved in bacteriophage budding from the host cell as a second site for protein fusion. Deleting gpIX from M13K07 is more complex than deleting gpIII since the coding sequence of gpIX overlaps with the coding sequence from gpVIII. Our design used to remove gpIX thus needed to include some gene refactoring to prevent interruption of the gpVIII gene. The overlapping sequence between gpVII and gpIX where the ATG codon is the start codon of gpIX and the TGA codon is the stop codon from gpVIII. The overlap between the two genes was corrected by mutating the A>G at position 3, changing the ATG codon to a weaker GTG start codon without affecting the sequence of gpVIII (both AGG and AGA encodes for arginine). Also, we changed the third codon of gpLX from TTA to TAA to introduce a stop codon and prevent the translation of gpIX. The resulting construct pTAT025 was next obtained by amplifying pTAT004 with primer introducing these modifications to the gpVIII/gpLX locus. Plasmid pTAT025 thus express all the genes of the M13 genome except for gpIII and gpIX, which needs to be provided in trans. It also needs a bacteriophage backbone vector encoding ori_{M13} to secrete bacteriophages. The same procedure was performed on pTAT032 to obtain pTAT032AgpIX, a pIX deficient bacteriophage secretion machinery displaying the anti-PDL1 nanobody on pIII.

Plasmid pTAT025 gpIII deficiency can be complemented by any of the plasmid described above that express gpIII or gpIII-therapeutic protein fusion (pTAT002, pTAT003, pTAT019, pTAT020, pTAT022, or pTAT030). However, pTAT025 also needs an exogenous supply of gpLX to produce bacteriophages. A set of plasmid was thus needed to support gpIX production. To this end, a new backbone was generated by amplifying ori_{pSC101} from pKN23, bla from pUC19 and P5-BCD1-sfGFP from pTAT010-P5 each of which are assembled using GAT in primer tails. The PCR fragments were next purified by SPRI and assembled by Gibson assembly generating pTAT014 before transformation in MM294. The construct was next evaluated phenotypically (GFP phenotype) and sequenced by Sanger sequencing. This backbone was next amplified in its entirety except for the sfGFP gene and was assembled with the gpLX gene amplified from M13K07. Both PCR products were next assembled in the same way as for pTAT014, generating a gpLX complementation plasmid (pTAT017). This plasmid was further modified to allow the display of the anti-CD47 nanobody (nbCD47) on pIX by amplifying all but P5-BCD1 and adding two DNA fragments originating from gBlock. The first one was a revTet expression system, and the second one was a pelB-nbCD47, cloned at the N-terminal of pIX. This produced plasmid pTAT028 after Gibson assembly and cloning in MM294. The plasmid was next confirmed by sanger sequencing. The pTAT017 vector was further modified to display the anticalin against CTLA-4, using primers to amplify the backbone of pTAT017, P5mut-BCD1 from pTAT010-P5mut and the gBlock_TAT10. Those primers also changed the start codon of the anticalin fusion protein from ATG to GTG. This construct was later named pTAT035 and allows display of the CTLA-4 anticalin on pIX.

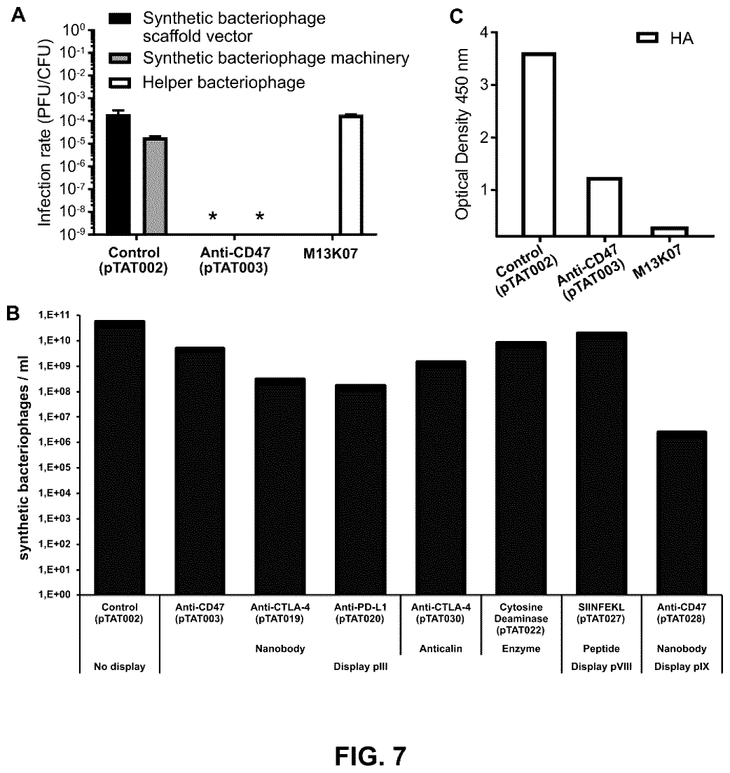

The first step towards biocontainment of the synthetic bacteriophage secretion system is to confine the synthetic bacteriophage machinery to the chromosome of the host cell. In a system where all components are extrachromosomic, bacteriophage particles can encapsidate either the synthetic bacteriophage backbone vector (90-99%) or by random error the synthetic bacteriophage machinery vector (1-10%) as seen in our tests (see FIG. 7A in example II). Insertion of the synthetic bacteriophage machinery in the chromosome of the host cell will result in the encapsidation of only the synthetic bacteriophage backbone vector. To mediate chromosomal integration of the synthetic bacteriophage machinery vector, a PCR amplification of pTAT004 (without the origin of replication and antibiotic resistance gene) was cloned between the att sites of a XhoI+NdeI digested pTAT00X. The two plasmids were fused together by Gibson assembly, purified by SPRI and cloned in electrocompetent EC100Dpir+ cells. Screening of plasmid clones was next performed by digestion using EcoRI and PvuII (FIG. 6). The completed new vector, called pTAT001, was next extracted from EC100Dpir+ and transformed into EC100Dpir++pTA-MOB. Using pTA-MOB conjugation machinery, pTAT001 was then mobilized by conjugation on agar medium from EC100Dpir+ to MG1655. The integration of the synthetic bacteriophage machinery was next induced with 1% arabinose for 2 hours at 30° C. and the plasmid backbone was lost by heatshock 1 hour at 42° C. before an overnight growth at 37° C. The resulting cells were then ready for transformation with pTAT002, pTAT003, pTAT019, pTAT020, pTAT022, or pTAT030 to complete the synthetic bacteriophage secretion system. Using pTAT01, bacteriophage particles are not be able to self-propagate if bacteria from the environment acquire this vector. This biocontainement level therefore constitutes an improved measure to avoid the spread of the engineered bacteriophage in the environment. The same strategy can be done to biocontain the synthetic bacteriophage machinery allowing the display of the therapeutic protein on pIX, or on other bacteriophage coating protein.

To further ameliorate the biocontainment of the synthetic bacteriophage secretion system, the therapeutic protein fused to pIII, or pIX, or to any bacteriophage coating protein, can be moved from pTAT002, pTAT003, pTAT019, pTAT020, pTAT022, pTAT028 or pTAT030 to the genome of the bacterial host cell. That way the therapeutic module is also integrated in the genome of the bacterial host cell. The synthetic bacteriophage backbone vector then only contains the ori_{M13}, a selection marker, and optionally a high copy number origin of replication. With the filler module, one can modify the length of the synthetic bacteriophage, a feature interesting for double specific phage particles applications (Specthrie et al., J. Mol. Biol., 1992, 3:720; incorporated herein by reference). For instance, when a binding protein fused to the tail of the bacteriophage binds to a cancer cell, and a binding protein fused to the head of the bacteriophage binds to a T-cell, the length of the bacteriophage influence the distance between the cancer cell and the T-cell. By modifying the size of the filler module, one can thus change the size of the synthetic bacteriophage and affect the distance between the cancer cell and the T-cel, and hence, influence the response of the T cell to the cancerous cell. Further modifications to the synthetic bacteriophage secretion system can ameliorate secretion and efficiency of the therapeutic activity. For example, using promoters that are inducible by environmental conditions only found in tumor microenvironment reduces possible side effects, or the genetic drifting of the live biotherapeutic during production scale up. This is illustrated by the pTAT028 construction, which is repressed by tetracycline. Splitting the synthetic bacteriophage machinery in multiple fragments that are inserted at distant loci in the genome of the bacterial host will also lower the chances of recombination. Using these constructions, the synthetic bacteriophage secretion system can display several proteins and peptides on different coating proteins (FIG. 2). Nonetheless, the different plasmids constructed above are transformed in E. coli MG1655 and combined to demonstrate the capacities of the synthetic bacteriophage secretion system.

### Example 2—Synthetic Bacteriophages are Secreted from the Live Biotherapeutic and Display Therapeutic Agents

All strains used in this Example are described in Table 1. Cells were typically grown in Luria broth Miller (LB) supplemented, when needed, with antibiotics at the following concentrations: kanamycin (Km)₅₀ μg/mL, spectinomycin (Sp) 100 μg/mL All cultures were routinely grown at 37° C. with agitation (200 rpm). No bacterial cultures over 18 hours of age were used in the experiments.

Polyethylene glycol-based precipitation of synthetic bacteriophage particles. Starting from frozen stock, inoculate 5 mL of sterile LB broth comprising the appropriate antibiotics at the concentrations specified in the paragraph above and incubate the culture at 37° C. overnight with agitation, or for no longer than 18 hours. Transfer 1.5 mL of the overnight bacterial culture and centrifuge for 2 minutes at 13000 g. Without disturbing the pellet, transfer 1.2 mL of the supernatant, comprising the bacteriophage particles, in a new sterile microtube. Add 300 μL of 2.5 M NaCl/20% PEG-8000 (w/v) to the culture supernatant (mix at a 4:1 supernatant:PEG solution volume ratio). After mixing thoroughly by inverting the tubes 15 times, the mixture is then incubated at 4° C. for 1 h. The virions are next pelleted by centrifugation 3 minutes at 13 000 g. The supernatant is then removed and the pellet resuspended with 120 μL of TBS 1× (Tris Buffer Saline: 50 mM Tris-HCl pH 7.5, 150 mM NaCl, sterile) corresponding to 1/10 of the initial culture volume. The bacteriophage preparation is kept on ice for another hour, vortexed, and used immediately afterwards.

Assessment of synthetic bacteriophage particles functionality by infection assay. To first test the integrity of the engineered bacteriophage particles, infection experiments were designed. A culture of E. coli ER2738 grew overnight in LB comprising the appropriate antibiotics. To test the infectivity of the bacteriophage particles, 1 μL of culture supernatent was added to 1 mL of E. coli ER2738. The mixture was next incubated at 37° C. for 1 h30 before plating for CFU analysis. Infected cells could be identified by the gain of either the spectinomycin resistance gene (synthetic bacteriophage backbone vector) or the kanamycin resistance gene (synthetic bacteriophage machinery vector or M13K07).

Synthetic bacteriophage titration by Enzyme Linked Immunosorbent Assay (ELISA). Detection and quantification of bacteriophage expression was performed using the commercially available Phage Titration ELISA kit (PRPHAGE, Progen) following manufacturer's instructions. Briefly, lyophilized M13 particles were resuspended at 1,5×10⁸ phage/mL as per manufacturer's recommendation and diluted 1/2 serially to generate a standard curve. Bacteriophage preparations were next diluted 1/1000 and 1/10000, and then added (as well as the standard curve) to mouse anti-M13 pre-coated ELISA wells. Bacteriophage particles captured were detected by a peroxidase conjugated monoclonal anti-M13. After addition of tetramethylbenzidine, the optical density of each well was measured at 450 nm using the Biotek plate reader instrument. The procedure was repeated using an antibody HA-Tag (6E2) Mouse mAb (HRP Conjugate) (1:1000 Cell Signaling Technology, Danvers, MA, USA) instead of the anti-M13-HRP provided with the kit to detect the modified pIII protein at the surface of engineered phages.

Phage infectivity assessment—Plasmids pTAT002 and pTAT003 were transformed in E. coli MG1655. The resulting strain was next transformed with pTAT004, creating MG1655+pTAT002+pTAT004 and MG1655+pTAT003+pTAT004 (Table 1). While the strain carrying pTAT002 should produce infectious phage particles (because pTAT002 carries a wildtype copy of the gpIII gene), the strain carrying pTAT003 should produce non-infectious phage particles, since wildtype gpIII is absent from pTAT004 and is fused to the anti-CD47 nanobody in pTAT003 (FIG. 3). To verify the infectivity of the bacteriophage particles, pTAT002, pTAT003 and M13K07 derived phages were purified using the PEG precipitation protocol. E. coli ER2738 cells were next infected with 1 μL of each phage preparation and incubated 1 h30 at 37° C. CFU were next quantified on LB agar plates selecting the host cells or the infected cells. Bacteriophage particles derived from pTAT002 and M13K07 were infectious, while phage particles derived from pTAT003 failed to infect cells, confirming that a complete copy of pIII is absent from pTAT003 (FIG. 7A). This result has two main implications. First, it shows that the synthetic bacteriophage secretion system produces functional phage particles when pIII is wild-type, therefore that all the genes implicated in synthetic bacteriophage phage machinery are working correctly. Second, it shows that the current configuration of the synthetic bacteriophage secretion system produces non-infectious phage particles when displaying a therapeutic protein on pIII. This is an important step towards the biocontainment of the system which shows that this system should not be able to infect and propagate by infecting natural hosts of M13.

Secretion of synthetic bacteriophage by the live biotherapeutic measured by ELISA—The first step to validate the integrity of synthetic bacteriophage secretion systems is to validate the secretion, by the bacterial host, of synthetic bacteriophages displaying various therapeutic proteins through various fusion. To achieve this, several iterations of synthetic bacteriophage secretion systems were assembled by transformation of different vector combinations in E. coli MG1655 producing different bacteriophages: control bacteriophages (MG1655+pTAT004+pTAT002), bacteriophages displaying an anti-CD47 nanobody on pIII (MG1655+pTAT004+pTAT003), bacteriophages displaying an anti-CTLA-4 nanobody on pIII (MG1655+pTAT004+pTAT019), bacteriophages displaying an anti-PD-L1 nanobody on pIII (MG1655+pTAT004+pTAT020), bacteriophages displaying the cytosine deaminase on pIII (MG1655+pTAT004+pTAT022), bacteriophages displaying an anti-CTLA-4 anticalin on pIII (MG1655+pTAT004+pTAT030), bacteriophages displaying an anti-CD47 nanobody on pIX (MG1655+pTAT025+pTAT002+pTAT028), and bacteriophages displaying an epitope from the chicken ovalbumine gene SIINFEKL on pVIII (pTAT027+pTAT002). Two types of ELISA assays were next performed on PEG precipitated bacteriophage particles from each of the synthetic bacteriophage secretion system iteration. The first ELISA assay was performed to detect the presence of pVIII, while the second ELISA assay was perform to detect the presence of the HA linker, which is present on pIII in bacteriophage particles derived from both pTAT002 and pTAT003, but not from M13K07. Phage preparations were diluted 1:1000 and all but one strain demonstrated a high signal in the anti-pVIII ELISA assay, which confirmed a high level of secretion of synthetic bacteriophages/mL (FIG. 7B). The strain displaying the anti-CD47 nanobody on pIX fusion produced lower bacteriophage count than other constructs. This lower efficiency might be linked to the expression system used for this construction, which differs from the others. The second ELISA confirmed the presence of the linker HA in both strains MG1655+pTAT004+pTAT002 (pIII HA) and MG1655+pTAT004+pTAT003 (anti-CD47 nanobody on pIII) (FIG. 7C). The anti-HA-HRP (Cell Signaling) antibody produced a signal only for the two modified systems expressing the HA tag, confirming the presence of the fusion pIII protein from the bacteriophage vector backbone in the bacteriophage particles. The strain MM294+M13K07 (pIII wild-type) did not show any signal in this assay as expected, since the HA tag linker is absent from pIII protein expressed by M13K07. This data support that the therapeutic protein are displayed.

### Example 3—the Synthetic Bacteriophage Displays Therapeutic Binding Proteins that can Recognize and Bind to Immune Checkpoints Expressed on Tumor Cells

All strains and plasmids used in this Example are described in Table 1. Cells were typically grown in Luria broth Miller (LB) or on Luria broth agar Miller medium supplemented, when needed, with antibiotics at the following concentrations: ampicillin (Ap) 100 μg/mL, chloramphenicol (Cm) 34 μg/mL, kanamycin (Km) 50 μg/mL, nalidixic acid (Nx) 4 μg/mL, spectinomycin (Sp) 100 μg/mL, streptomycin (Sm) 50 μg/mL, sulfamethoxazole (Su) 160 μg/mL, tetracycline (Tc) 15 μg/mL, and trimethoprim (Tm) 32 μg/mL. All cultures were routinely grown at 37° C. for no longer than 18 hours before use in the experiments. Bacteriophages were extracted from confluent bacterial culture (grown overnight) using the PEG precipitation protocol presented in example II. The bacteriophage preparation was used immediately after precipitation. A20 lymphocyte B lymphoma cells were ordered from ATCC (TIB-208). Upon arrival, cells were washed and resuspended in RPMI-1640 supplemented with 10% Fetal Bovine Serum (FBS) and 0.05 mM 2-mercaptoethanol. This culture medium was used for the preparation of cells for all experiments. A frozen stock was generated after 4 passages and was used to start subsequent cultures for experimentations. Cells were maintained at density between 2×10⁵ cell/mL and 2×10⁶ cell/mL throughout all the experiments.

Synthetic bacteriophages pull down assay. PEG precipitated bacteriophages were resuspended in Phosphate Buffered Saline (PBS)+Bovine Serum Albumine (BSA) 0.2% p/v (PBS-B) and incubated at 4° C. for 1 hour. 1 mL of cells culture at a density of approximatively 1×10⁶ cell/mL was centrifuged 3 min at 400 g and resuspended in 500 μL of PBS-B. Cells were centrifuged again at 400 g for 3 minutes before resuspension in 100 μL of bacteriophage solution. An aliquot of the cell bacteriophage mixture was saved for further analysis. Next, the cells were washed 6 times in PBS-B and at the first, third and sixth wash, an aliquot of 20 μL was kept on ice. After washing the cells 6 times, 10 μL of all aliquot were mixed with 90 μL of 5% p/v Chelex beads in a PCR tube. DNA was extracted by incubating the mix at 50° C. for 25 minutes and 100° C. for 10 minutes. The DNA preparations were amplified by qPCR using the TransStart PFU fly DNA polymerase kit (Civic Bioscience) supplemented with EvaGreen dye (Biotium). Bacteriophage DNA was amplified using primers oTAT043 and oTAT044 described in Table 2. The other 10 μL for all aliquots was diluted with 90 μL of PBS and used to assess cellular count.

Assessment of synthetic bacteriophage binding to therapeutic target by flow cytometry. PEG precipitated bacteriophages were resuspended in Phosphate Buffered Saline (PBS)+Bovine Serum Albumine (BSA) 0.2% p/v (PBS-B) and incubated at 4° C. for 1 hour. 1 mL of cells at a density of approximatively 1×10⁶ cell/mL were centrifuged at 400 g for 3 minutes and resuspended in 500 μL of PBS-B. Cells were centrifuged again at 400 g for 3 minutes before resuspension in 100 μL of the therapeutic bacteriophage solution displaying a nanobody, or in 100 μL PBS-B for the no bacteriophage control. Cells and bacteriophages were incubated 1 hour at 4° C. Then, the mixture was centrifuged at 400 g for 3 minutes. The cells were then resuspended in 50 μL PBS-B comprising 1 μg of miap301 FITC anti-CD47 rat IgG2a (Biolegend) when assessing the specificity of the anti-CD47 nanobody, or in 50 μL PBS-B comprising 0.25 μg of PE anti-CD274 (B7-H1, PD-L1) rat IgG2b (Biolegend) when assessing the specificity of the anti-PD-L1 nanobody. A control group without staining was also prepared the same way except without labeling the cells with antibodies. Cells were incubated 30 minutes at 4° C. in the dark, then centrifugated at 400 g for 3 minutes, and finally resuspended in 500 μL of PBS. Cells were next analysed on a BD Accuri C6 Plus, or BD FACSJazz™ Cell Sorter, flow cytometers.

Assessment of synthetic bacteriophage binding to CTLA-4 by EUSA. To measure the binding activity of a synthetic bacteriophage directed against the checkpoint CTLA-4, an ELISA assay was devised. A 96 well plate was first coated overnight at 4° C. with recombinant CTLA-4 protein (R&D systems) diluted at a 10 μg/mL in coating buffer (0.05 M Carbonate-Bicarbonate at pH 9.6). The plate was then washed 3 times with 200 μL of TBS-T. To prevent unspecific binding, the plate was subsequently incubated with 200 μL of blocking buffer (TBS-T, 3% skimmed milk, 1% BSA) 1 hour at RT. Blocking was stopped by removing the blocking buffer and washing the plate two times with 200 μL of TBS-T. Then, 100 μL of PEG precipitated synthetic bacteriophages diluted in the TBS 1× and displaying either an anti-CTLA-4 nanobody (pTAT004+pTAT019), an anti-CTLA-4 anticalin (pTAT004+pTAT030) or a wildtype pIII (control: pTAT004+pTAT002), were added to wells comprising the CTLA-4 protein, or not, and incubated for 1 h at RT. The plate was then washed 3 times with 200 μL of TBS-T and 100 μL of Anti-pVIII-HRP (anti-M13/fd/F1, B62-FE2) diluted in blocking buffer (1:500) were added. The plate was incubated 1 h at RT in the dark and then washed 5 times with TBS-T. To measure the presence of the synthetic bacteriophages, 100 μL of TMB substrate solution (ThermoFisher) was added to each well and the plate was incubated 15 min at RT. The reaction was stopped by adding 100 μL of stop solution (0.5 M H₂SO₄) to each well. The absorbance was then measured at 450 nm.

Assessment of anti-PD-L1 displaying bacteriophages binding activity on A20 cells by EUSA. A 96 wells plate was prepared by adding in each well 100 μL of PBS comprising 1×10⁵ A20 cells. The plate was then incubated 30 min to allow sedimentation of the cells. The plate was then tilted and the PBS was carefully removed. To fix the cells to the plate, 100 μL of 10% formalin was then added, and the plate was incubated for 10 min. The fixed cells were then washed gently with 100 μL of PBS and then blocked by adding 200 μL of PBS (1% BSA, 3% milk), followed a 30 min incubation periode. The blocking buffer was removed and then 100 μL of bacteriophage PEG preparation was added to the wells. The plate was incubated 1 h and then washed three times with 200 μL of PBS. To detect the bacteriophages, 100 μL of anti-HA-HRP (Cell Signaling) 1/500 diluted in PBS (3% milk, 1% BSA) was added and the plate was incubated for 1 h. The wells were washed three times with 200 μL of PBS and then 100 μL of Tetramethylbenzidine (TMB) substrate solution was added to each well. The plate was incubated 5 min and then 100 μL of stop solution were added. Then, 100 μL from each well was transferred to a new plate and optical density was measured at 450 nm.

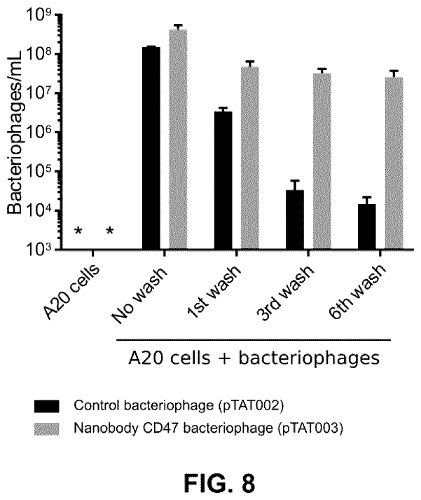

The live biotherapeutic secrete synthetic bacteriophage displaying the anti-CD47 nanobody, which binds to the surface of A20 cells. This example aims to confirm the capacity of the synthetic bacteriophage produced by the live biotherapeutic to bind to CD47 on the surface of A20 mouse lymphoma cells. Control bacteriophages (MG1655+pTAT004+pTAT002), or bacteriophages displaying an anti-CD47 nanobody on pIII (MG1655+pTAT004+pTAT003) were prepared in biological triplicate. 100 μL of bacteriophage preparations comprising 10⁹ particles were next mixed with 1,5×10⁶ A20 cells and incubated 1 hour at 4° C. Cells were then washed 6 times to get rid of phage particles that did not bind specifically to A20 cells. An aliquot of the cell mix was then analysed by qPCR to quantify the number of phage particles present at the mixing step, the first wash, the third wash and the sixth wash (FIG. 8). Results show that the bacteriophage particles produced with pTAT002 (which do not express the nanobody against CD47) are quickly washed from the cells while pTAT003 derived bacteriophages binds to the A20 cells and very few are lost by the washing procedure apart from the excess phages at the first wash step. These results show that the bacteriophage particles displaying the pIII-anti-CD47 nanobody fusion strongly bind to the target on cancerous cells, most likely through the binding to the CD47 receptor.

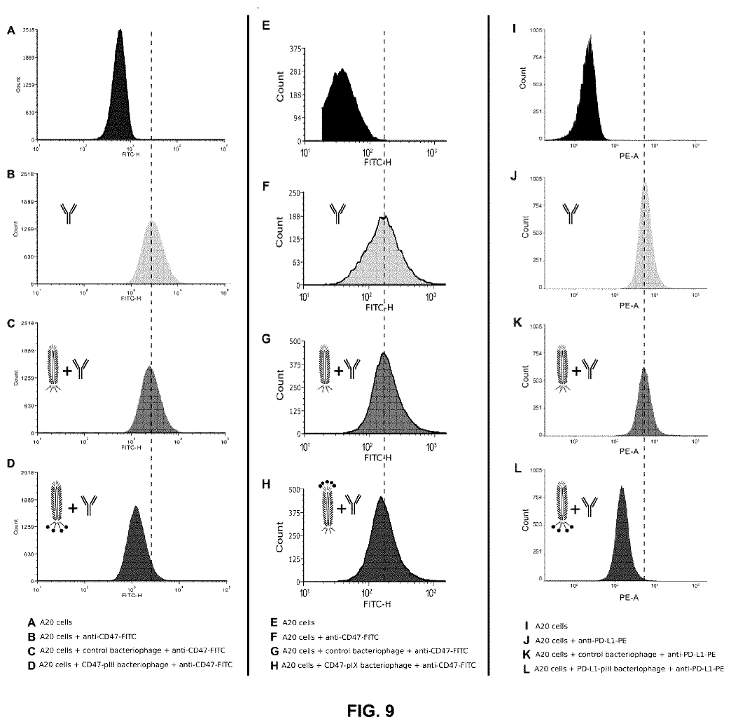

The live biotherapeutic secretes a synthetic bacteriophage displaying anti-CD47 on pIII or pIX, which binds specifically to CD47 receptors at the surface of the tumor cells. To confirm that the synthetic bacteriophage binds specifically to CD47 on the surface of A20 cells, a flow cytometry experiment was performed. In this experiment, A20 cells were first incubated with either PBS-B, control bacteriophages (MG1655+pTAT004+pTAT002), or bacteriophages displaying the anti-CD47 nanobody on pIII (MG1655+pTAT004+pTAT003) to allow the bacteriophage to bind the CD47 on the surface of the cells. Then, the cells were washed and incubated with an anti-CD47-FITC (miap301, Biolegend) antibody. Specific binding of the synthetic bacteriophage to CD47 would therefore result in decreased binding of the anti-CD47-FITC antibody, and hence, in a reduced the FITC signal. The experiment comprised four groups. The first group consisted in the A20 cells alone, which is a negative control to measure the background fluorescence signal (FIG. 9A). The second group consisted in A20 cells incubated only with the anti-CD47-FITC antibody, which is a positive control for the fluorescence signal (FIG. 9B). The third group comprised the A20 cells first incubated with synthetic bacteriophage derived from MG1655+pTAT004+pTAT002 (control) and then incubated with the anti-CD47-FITC antibody (FIG. 9C). The last group comprised the A20 cells incubated with synthetic bacteriophage particles derived from MG1655+pTAT004+pTAT003 (displaying the anti-CD47 nanobody on pIII), and then incubated with the anti-CD47-FITC antibody (FIG. 9D). Using the first two group as references for untagged and tagged population, the impact of the bacteriophage on the binding of the anti-CD47-FITC antibody was evaluated. Only the bacteriophages derived from pTAT003 (displaying the anti-CD47 nanobody) were able to hide the CD47 epitope recognized by the antibody, reducing the binding of the anti-CD47 antibody, therefore decreasing the FITC signal. The experiment was next repeated using synthetic bacteriophage particles derived from MG1655+pTAT025+pTAT002+pTAT028 (displaying the anti-CD47 nanobody on pIX) (FIG. 9E-H). The synthetic bacteriophage displaying the anti-CD47 nanobody on pIX showed a lower shift in fluorescence as compared with the synthetic bacteriophage displaying the anti-CD47 nanobody on pIII. The lower shift observed is linked to the lower secretion levels for this construction as discussed in example II. These results show that the synthetic bacteriophages derived from MG1655+pTAT004+pTAT003 and from MG1655+pTAT025+pTAT002+pTAT028 specifically bind to the CD47 receptor on the surface of the A20 cells. Therefore, the live biotherapeutic can produce functional bacteriophage particles that display an anti-CD47 nanobody, on pIII or pIX, capable of binding to the CD47 receptors on A20 lymphoma cells. The displayed protein can be harbored by both the bacteriophage head and tail proteins without discrimination. CD47 being an important immune checkpoint, preventing its binding to T-cells receptor should trigger an immune response against the tumor cells.

The live biotherapeutic secretes bacteriophages displaying anti-PD-L1 that binds specifically to PD-L1 receptors at the surface of the tumor cells. The synthetic bacteriophage can display different functional checkpoint inhibitors. To demonstrate that a synthetic bacteriophage displaying an anti-PD-L1 nanobody binds specifically to PD-L1 on the surface of A20 cells, a flow cytometry experiment was performed. In this experiment, A20 cells were first incubated with PBS-B, or phage particles derived from pTAT002 (control phage) or pTAT020 (phage displaying an anti-PD-L1 nanobody on pIII). Then, the cells were washed and incubated with an anti-PD-L1-PE (10F.9G2, Biolegend) antibody. Specific binding of the synthetic bacteriophage to PD-L1 would therefore result in decreased binding of the anti-PD-L1-PE antibody, and hence, in a reduced the PE signal. The experiment comprised four groups. The first group consisted of unstained A20 cells, which is a negative control to measure the background fluorescence signal (FIG. 9I). The second group consisted of A20 cells incubated only with the anti-PD-L1-PE antibody, which is a positive control for the fluorescence signal (FIG. 9J). The third group comprised the A20 cells first incubated with synthetic bacteriophage derived from MG1655+pTAT004+pTAT002 (control) and then incubated with the anti-PD-L1-PE antibody (FIG. 9K). Finally, the last group comprised the A20 cells incubated with synthetic bacteriophage particles derived from MG1655+pTAT004+pTAT020 (displaying the anti-PD-L1 nanobody), and then incubated with the anti-PD-L1-PE antibody (FIG. 9L). Using the first two groups as references for untagged and tagged populations, the impact of the bacteriophage on the binding of the anti-PD-L1-PE antibody was evaluated. Only the bacteriophages derived from pTAT020 (displaying the anti-PD-L1 nanobody) were able to hide the PD-L1 epitope recognized by the antibody, reducing the binding of the anti-PD-L1 antibody, therefore decreasing the PE signal. This shows that the synthetic bacteriophages derived from MG1655+pTAT004+pTAT020 specifically bind to the PD-L1 receptor on the surface of the A20 cells. Therefore, these results support that the live biotherapeutic can produce functional bacteriophage particles that display checkpoint inhibitors, such as an anti-PD-L1 nanobody capable of binding to the PD-L1 receptors on A20 lymphoma cells. PD-L1 being an important immune checkpoint, preventing its binding to T-cells receptor should trigger an immune response against the tumor cells.

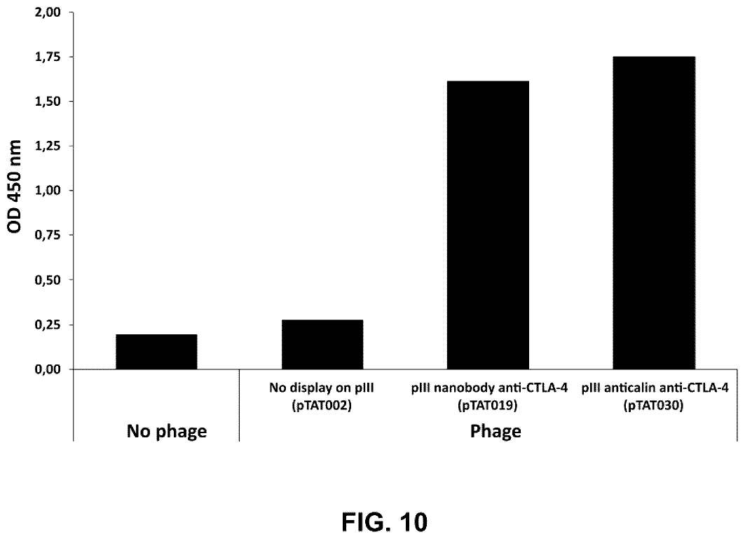

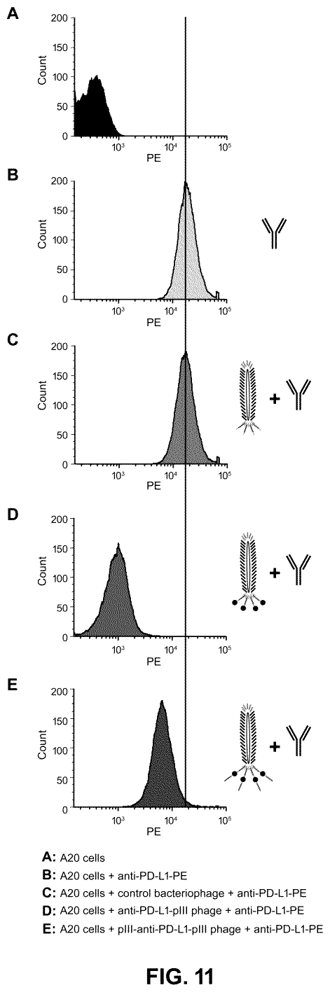

The live biotherapeutic secretes synthetic bacteriophages displaying anti-CTLA-4 binding proteins on pIII which binds specifically to the CTLA-4 immune checkpoint. Previous constructions demonstrated that bacteriophages can display functional nanobodies that can recognizes different targets at the surface of tumor cells. The synthetic bacteriophage system can also display proteins that recognize receptors specific to immune cells, such as CTLA-4. To demonstrate that the binding protein displayed on the bacteriophage can be of different types, bacteriophages were designed to display an anti-CTLA-4 nanobody (MG1655+pTAT004+pTAT019) or an anti-CTLA4 anticalin (MG1655+pTAT004+pTAT030). To demonstrate that synthetic bacteriophages displaying anti-CTLA-4 nanobody and anticalin proteins bind specifically to their target protein, an ELISA experiment was performed. In this experiment, synthetic bacteriophages derived from either pTAT002 (control with no display on pIII), pTAT019 (pIII display of an anti-CTLA-4 nanobody), or pTAT030 (pIII display of an anti-CTLA-4 anticalin) were incubated in 96 well plates with wells coated with mice CTLA-4 recombinant protein. Once the binding step was completed, the 96 well plates were washed with TBS-T and then incubated with anti-pVIII-HRP B62-FE3 (progen). This step reveals if synthetic bacteriophages are bound to their target by tagging the bacteriophage's pVIII protein with horseradish peroxidase. The presence of synthetic bacteriophage bound to their target is measured by adding TMB substrate, which produces a signal at 450 nm when TMB is oxidized by the activity of the horseradish peroxidase enzyme. A signal is only measured with the synthetic bacteriophages displaying an anti-CTLA-4 binding protein, which proves that synthetic bacteriophages can be used to target an immune checkpoint using different types of binding proteins (FIG. 10). The live biotherapeutic secretes bacteriophages with functional therapeutic protein inserted within a split functional coating protein. To demonstrate that therapeutic proteins can successfully be displayed when inserted in the middle of a phage coating proteins, the anti-PD-L1 nanobody was inserted between the D1/D2 domains and the transmembrane region of pIII (see FIG. 5 pTAT033). As a control, the anti-PD-L1 nanobody was also cloned at the N-terminal of pIII in a similar way as in pTAT020, but directly in the bacteriophage secretion machinery (see FIG. 5 pTAT032). A flow cytometry experiment was then performed to assess the binding activity of the corresponding synthetic bacteriophages. In this experiment, A20 cells were first incubated either with a control synthetic bacteriophage (pTAT002, no display) or the synthetic bacteriophage that displays the anti-PD-L1 nanobody on pIII (pTAT032 and pTAT033) to allow the bacteriophage to bind the PD-L1 on the surface of the cells. Then, the cells were washed and incubated with an anti-PD-L1-PE (10F.9G2, Biolegend) antibody. Specific binding of the synthetic bacteriophage to PD-L1 would therefore result in decreased binding of the anti-PD-L1-PE antibody, and hence, in a reduced the PE signal. The experiment comprised five groups. The first group consisted of the A20 cells alone, which is a negative control to measure the background fluorescence signal (FIG. 11A). The second group consisted of A20 cells incubated only with the anti-PD-L1-PE antibody, which is a positive control for the fluorescence signal (FIG. 11B). The third group comprised the A20 cells first incubated with synthetic bacteriophage derived from MG1655+pTAT004+pTAT002 (control) and then incubated with the anti-PD-L1-PE antibody (FIG. 11C). The forth group comprised the A20 cells incubated with synthetic bacteriophage particles derived from MG1655+pTAT32 (displaying the anti-PD-L1 nanobody inserted at the N-terminal of pIII), and then incubated with the anti-PD-L1-PE antibody (FIG. 11D). Finally, the last group comprised the A20 cells incubated with synthetic bacteriophage particles derived from MG1655+pTAT033 (displaying the anti-PD-L1 nanobody inserted in pIII), and then incubated with the anti-PD-L1-PE antibody (FIG. 11E). Using the first two groups as references for untagged and tagged populations, the impact of the bacteriophage on the binding of the anti-PD-L1-PE antibody was evaluated. Only the bacteriophages derived from pTAT032 and pTAT033 were able to hide the PD-L1 epitope recognized by the antibody, which reduced the binding of the anti-PD-L1 antibody, therefore similarly decreasing the PE signal. This shows that the synthetic bacteriophages derived from MG1655+pTAT033 specifically bind to the PD-L1 receptor on the surface of the A20 cells and that a functional therapeutic protein can be inserted in a coating protein and displayed properly. The pTAT033 construction also shows that proteins larger than the size of a nanobody could be displayed on bacteriophage pIII coating protein and retain their function. The size of the pTAT033 pIII fusion protein is equivalent to two nanobodies, which, if cloned on pill, could both bind different targets. Furthermore, the pTAT033 derived phage particles remained infectious, which supports that both the nanobody and the N-Terminal part of pIII kept the function, showing that two binding functional binding proteins can be cloned on the same coat protein.

### Example 4—Synthetic Bacteriophages that Display Peptides on PVIII

All strains and plasmids used in this Example are described in Table 1. Cells were typically grown in Luria broth Miller (LB) or on Luria broth agar Miller medium supplemented, when needed, with antibiotics at the following concentrations: ampicillin (Ap) 100 μg/mL, chloramphenicol (Cm) 34 μg/mL, kanamycin (Km) 50 μg/mL, nalidixic acid (Nx) 4 μg/mL, spectinomycin (Sp) 100 μg/mL, streptomycin (Sm) 50 μg/mL, sulfamethoxazole (Su) 160 μg/mL, tetracycline (Tc) 15 μg/mL, and trimethoprim (Tm) 32 μg/mL. All cultures were routinely grown at 37° C. for no longer than 18 hours before use in the experiments. Bacteriophages were extracted from confluent bacterial culture (grown overnight) using the PEG precipitation protocol presented in example II. The bacteriophage preparation was used immediately after precipitation. A detailed list of oligonucleotide sequences used in this Example is found in Table 2. Plasmids were prepared using EZ10-Spin Column Plasmid Miniprep kit (BIOBASIC #BS614) or QIAGEN Plasmid Maxi Kit (QIAGEN) according to the manufacturer's instructions. PCR amplifications were performed using TransStart FastPFU fly DNA polymerase (Civic Bioscience) for DNA parts amplification and screening. Digestion with restriction enzymes used products from NEB and were incubated for 1 hour at 37° C. following manufacturer's recommendations. Plasmids were assembled by Gibson assembly using the NEBuilder HiFi DNA Assembly Master Mix (NEB) following manufacturer's protocol. Sanger sequencing reactions were performed by the Plateforme de séquençage de l'Université Laval.

DNA purification. Purification of DNA was performed between each step of plasmid assembly to avoid buffer incompatibility or to stop enzymatic reactions. PCR reactions were generally purified by Solid Phase Reversible Immobilization (SPRI) using Agencourt AMPure XP DNA binding beads (Beckman Coulter) according to the manufacturer's guidelines or recovered and purified from agarose gel using Zymoclean Gel DNA Recovery Kit (Zymo Research). When DNA samples were digested with restriction enzymes, DNA was purified using Monarch® PCR & DNA Cleanup Kit (NEB) following manufacturer's recommendation for cell suspension DNA purification protocol. After purification, DNA concentration and purity were routinely assessed using a Nanodrop spectrophotometer when necessary.

Cell culture. A20 lymphocyte B lymphoma cells were ordered from ATCC (TIB-208). Upon arrival, cells were washed and resuspended in RPMI-1640 supplemented with 10% Fetal Bovine Serum (FBS) and 0.05 mM 2-mercaptoethanol. This culture medium was used for the preparation of cells for all experiments. A frozen stock was generated after 4 passages and was used to start subsequent cultures for experimentations. Cells were maintained at density between 2×10⁵ cell/mL and 2×10⁶ cell/mL throughout all the experiments.

Synthetic bacteriophage titration by Enzyme Linked Immunosorbent Assay (ELISA). Detection and quantification of bacteriophage expression was performed using the commercially available Phage Titration ELISA kit (PRPHAGE, Progen) following manufacturer's instructions. Briefly, lyophilized M13 particles were resuspended at 1,5×10¹ phage/mL as per manufacturer's recommendation and diluted 1/2 serially to generate a standard curve. Bacteriophage preparations were next diluted 1/10, 1/100, 1/1000, and 1/10000 and added (as well as the standard curve) to mouse anti-M13 pre-coated ELISA wells. Bacteriophage particles captured were detected by a peroxidase conjugated monoclonal anti-M13. After addition of tetramethylbenzidine the optical density of each well was measured at 450 nm using the Biotek plate reader instrument. The procedure was repeated using an antibody HA-Tag (6E2) Mouse mAb (HRP Conjugate) (1: 1000 Cell Signaling Technology, Danvers, MA, USA) instead of the anti-M13-HRP provided with the kit to detect the modified pIII protein at the surface of engineered phages.

Verification of fusion protein integrity by western blot. Bacteria were grown overnight at 37° C. with agitation in LB broth supplemented with kanamycin and spectinomycin. The bacteria were pelleted by centrifugation and the culture supernatants were transferred in a new tube and buffered with concentrated PBS. The phages displaying an hexahistidine tag were pulldown from the supernatants using Ni-NTA beads by incubating 2 hours at 4° C. with agitation. The beads were then washed 3 times with PBS and the phages were eluted by denaturation using sample buffer 4X (SB4X). The samples were denatured for 1 hour at 65° C. and loaded on a 15% acrylamide gel. The gel migration of the samples was performed for 1 hour at 150 volts. The proteins were then transferred on a 0.2 μm nitrocellulose membrane by applying 100 volts for 1 hour. The membrane was air dried to let evaporate any trace of methanol and blocked for 1 hour in TBS—0.1% Tween 20 —4% dried milk at 4° C. with agitation. The membrane was transferred in a western blot sealable bag and incubated over night with anti-HA-HRP (Cell Signaling) diluted in blocking buffer at 4° C. with agitation. After three TBS—0.1% Tween 20 washes, the membrane revelation was performed by applying the Immobilon ECL Ultra Western HRP substrate and the image acquired with the Vilber Fusion FX apparatus. The images were processed using the Image Lab software.

Assessment of synthetic bacteriophage binding to therapeutic target by flow cytometry. PEG precipitated bacteriophages were resuspended in Phosphate Buffered Saline (PBS)+Bovine Serum Albumine (BSA) 0.2% p/v (PBS-B) and incubated at 4° C. for 1 hour. 1 mL of cells at a density of approximatively 1×10⁶ cell/mL were centrifuged at 400 g for 3 minutes and resuspended in 500 μL of PBS-B. Cells were centrifuged again at 400 g for 3 minutes before resuspension in 100 μL of the therapeutic bacteriophage solution displaying a nanobody, or in 100 μL PBS-B for the no bacteriophage control. Cells and bacteriophages were incubated 1 hour at 4° C. Then, the mixture was centrifuged at 400 g for 3 minutes. The cells were then resuspended in 50 μL PBS-B comprising 1 μg of miap301 FITC anti-CD47 rat IgG2a (Biolegend) when assessing the specificity of the anti-CD47 nanobody, or in 50 μL PBS-B comprising 0.25 μg of PE anti-CD274 (B7-H1, PD-L1) rat IgG2b (Biolegend) when assessing the specificity of the anti-PD-L1 nanobody. A control group without staining was also prepared the same way except without labeling the cells with antibodies. Cells were incubated 30 minutes at 4° C. in the dark, then centrifugated at 400 g for 3 minutes, and finally resuspended in 500 μL of PBS. Cells were next analysed for using the FITC channel of a BD Accuri C6 Plus flow cytometer.

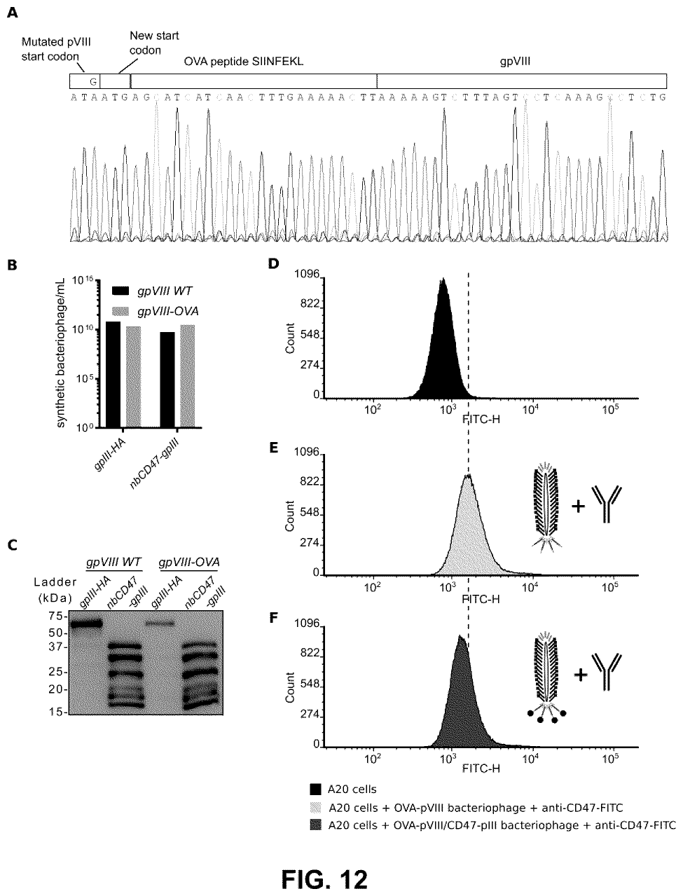

The synthetic bacteriophage secretion system produces bacteriophages displaying peptides on the major coat protein pVIII. To validate that the bacteriophage secretion machinery pTAT027 (see Example I) displays a peptide from the chicken ovalbumine gene on pVIII (pVIII-OVA), the corresponding region of the construction was sequenced by sanger (FIG. 12A). The OVA peptide is in frame with the pVIII protein, as such, if the pVIII protein can be detected with an antibody, the OVA peptide necessarily present at the surface of the bacteriophage particles. The pTAT027 machinery, which is pIII deficient, was thus complemented with either pTAT002 (providing an HA-tagged pIII) or pTAT003 (providing an anti-CD47-nanobody-HA-pIII) in E. coli MG1655. The strain were then grown overnight and bacteriophages were purified by PEG precipitation. Bacteriophage production was next detected by ELISA (kit PRPHAGE Progene) MG1655+pTAT004+pTAT002 and MG1655+pTAT004+pTAT003 as controls not displaying the OVA peptide (FIG. 12B). Synthetic bacteriophage secretion systems displaying the OVA peptide on pVIII produced similar amount of bacteriophages as their counterparts with wildtype pVIII, suggesting that the display of peptides on pVIII do not hinder bacteriophage production. To confirm that the bacteriophage display either the HA-tagged pIII protein fusion (pTAT002) or the nbCD47-HA-pIII protein fusion (pTAT003) regardless of the display of OVA peptides on pVIII, bacteriophages were purified with Ni-NTA and analysed by western blot, revealing proteins using an anti-HA-HRP (Cell Signaling) antibody (FIG. 12C). The bacteriophages displaying the OVA peptides on pVIII produced similar pattern as the control bacteriophages for their pIII display, suggesting that bacteriophage assembly could be completed in both cases and produced complete bacteriophage particles.

The synthetic bacteriophage secretion system produces bacteriophage displaying peptides on pVIII and a functional binding protein on pIII that hides immune checkpoint on the surface of cancer cells. To confirm that the synthetic bacteriophage displaying the OVA peptide on pVIII do not compromise the integrity of proteins displayed on pIII, the functionality of synthetic bacteriophage displaying both OVA on pVIII and the anti-CD47 nanobody on pIII was assessed. To verify the binding of bacteriophages to CD47 on the surface of A20 cells, a flow cytometry experiment was performed. In this experiment, the A20 cells were first incubated with either PBS-B, the phage particles derived from MG1655+pTAT027+pTAT002 (control pVIII-OVA alone) or the phage particles derived from MG1655+pTAT027+pTAT003 (peptide OVA displayed on pVIII and anti-CD47 nanobody displayed on pIII) to allow the bacteriophage to bind to CD47 on the surface of cells. Then, the cells were washed and incubated with an anti-CD47-FITC (miap301, Biolegend) antibody. Specific binding of the synthetic bacteriophage to CD47 would therefore result in decreased binding of the anti-CD47-FITC antibody, and hence, in a reduced the FITC signal. The experiment comprised three groups. The first group consisted in the A20 cells alone, which is a negative control to measure the background fluorescence signal (FIG. 12D). The second group comprised the A20 cells first incubated with synthetic bacteriophage derived from MG1655+pTAT027+pTAT002 (control, OVA on pVIII alone) and then incubated with the anti-CD47-FITC antibody (FIG. 12E). The last group comprised the A20 cells incubated with synthetic bacteriophage particles derived from MG1655+pTAT027+pTAT003 (displaying OVA on pVIII and the anti-CD47 nanobody on pIII), and then incubated with the anti-CD47-FITC antibody (FIG. 12F). Using the first two group as references for untagged and tagged population, the impact of the bacteriophages on the binding of the anti-CD47-FITC antibody was evaluated. Similarly to the experiments shown in FIG. 9, only the bacteriophages derived from pTAT003 (displaying the anti-CD47 nanobody) were able to hide the CD47 epitope recognized by the antibody, which reduces the binding of the anti-CD47 antibody, therefore decreasing the FITC signal. The live biotherapeutic can thus produce functional bacteriophage particles that display an anti-CD47 nanobody capable of binding to the CD47 receptors on A20 lymphoma cells while displaying a peptides on pVIII. CD47 being an important immune checkpoint, preventing its binding to T-cells receptor should trigger an immune response against the tumor cells.

### Example 5—Synthetic Bacteriophages that Display Checkpoint Inhibitors have a Direct Anti-Tumoral Effect

All strains and plasmids used in this Example are described in Table 1. Cells were typically grown in Luria broth Miller (LB) or on Luria broth agar Miller medium supplemented, when needed, with antibiotics at the following concentrations: ampicillin (Ap) 100 μg/mL, chloramphenicol (Cm) 34 μg/mL, kanamycin (Km) 50 μg/mL, nalidixic acid (Nx) 4 μg/mL, spectinomycin (Sp) 100 μg/mL, streptomycin (Sm) 50 μg/mL, sulfamethoxazole (Su) 160 μg/mL, tetracycline (Tc) 15 μg/mL, and trimethoprim (Tm) 32 μg/mL. All cultures were routinely grown at 37° C. for no longer than 18 hours before use in the experiments. Bacteriophages were extracted from confluent bacterial culture (grown overnight) using the PEG precipitation protocol presented in Example 2. A20 lymphocyte B lymphoma cells were ordered from ATCC (TIB-208). Cells were maintained at densities between 2×10^{W} cell/mL and 2×10⁶ cell/mL throughout all the experiments in RPMI-1640 supplemented with 10% Fetal Bovine Serum (FBS) and 0.05 mM 2-mercaptoethanol.

PD-L1 nanobody protein production and purification. The coding sequence of the anti-PD-L1 nanobody with hexahistine and HA tags fused in C-terminal was cloned in the pTrcHis vector by Gibson assembly. The resulting plasmid was transformed in BL21 (DE3) competent E. coli and the transformants were selected on LB plates with ampicillin. The plasmids were extracted from the transformants and the integrity of the nanobody coding sequence was confirmed by sanger sequencing. For protein expression and purification, the BL21 transformants were cultivated in LB with ampicillin. The protein expression was induced by adding 1 mM of IPTG followed by an 18 hours incubation at room temperature with agitation. The protein purification was performed by incubating the cell lysate with Ni-NTA agarose beads (Qiagen) for 18 hours at 4° C. The proteins were eluted by incubating the Ni-NTA agarose beads with 200 mM of imidazole. The proteins were then concentrated from the eluate using a Amicon UItra-15 10 kDa Centrifugal Filter Unit to a final volume of 500 μL. The concentrated proteins were resuspended in sterile PBS to a final volume of 15 mL. The cycle of concentration and resuspension was repeated 3 times. Following the final concentration step, the protein purity was verified by spectrophotometry and SDS-PAGE. The functionality of the purified and concentrated anti-PD-L1 nanobody was confirmed by ELISA on A20 cells expressing PD-L1.

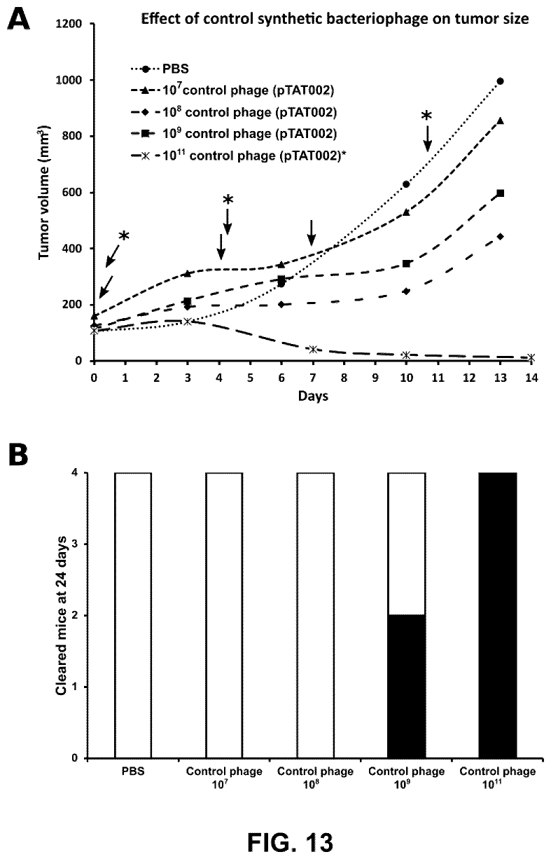

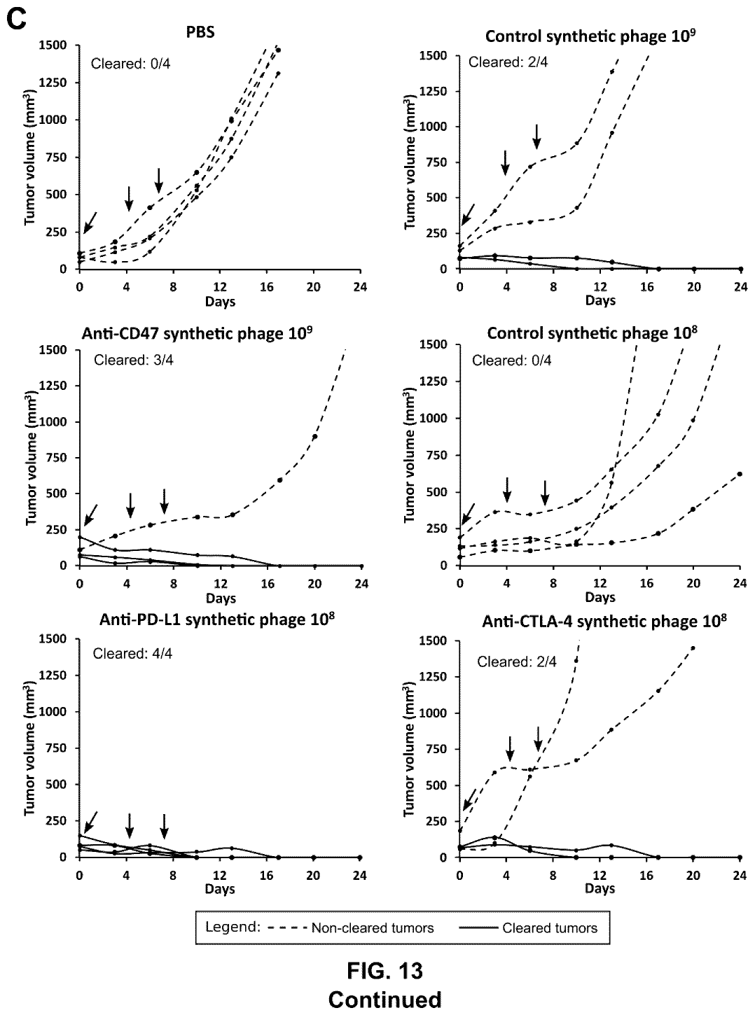

Assessment of anti-PD-L1 nanobody binding activity on A20 cells by EISA. A 96 wells plate was prepared by adding in each well 100 μL of PBS comprising 1×10⁵ A20 cells. The plate was then incubated 30 min to allow sedimentation of the cells. The plate was then tilted and the PBS was carefully removed. To fix the cells to the plate, 100 μL of 10% formalin was then added, and the plate was incubated for 10 min. The fixed cells were then washed gently with 100 μL of PBS and then blocked by adding 200 μL of PBS (1% BSA, 3% milk) followed a 30 min incubation periode. The blocking buffer was removed and then 100 μL of nanobody was added to the wells in concentration ranging from 1 nM to 10 μM. The plate was incubated 1 h and then washed three times with 200 μL of PBS. To detect the nanobody, 100 μL of anti-HA-HRP (Cell Signaling) 1/500 diluted in PBS (3% milk, 1% BSA) was added and the plate was incubated for 1 h. The wells were washed three times with 200 μL of PBS and then 100 μL of Tetramethylbenzidine (TMB) substrate solution was added to each well. The plate was incubated 5 min and then 100 μL of stop solution were added. Then, 100 μL from each well was transferred to a new plate and optical density was measured at 450 nm. High doses of control synthetic bacteriophage exhibit strong anti-tumoral effects. To assess whether the control synthetic bacteriophage alone, i.e with no therapeutic protein displayed, can have an anti-tumoral effect, mice were first injected subcutaneously with 5×10⁶ A20 cells in their right flanks and were observed every two days to monitor tumor growth. Mice were next divided into five treatment groups. The first group received 50 μL of PBS (vehicle control). The remaining groups received 50 μL of PBS comprising increasing doses of PEG purified control synthetic bacteriophage (pTAT002) 107, 10⁸, 10⁹, and 10¹⁰ bacteriophage particles. For PBS, 107, 10′, and 10⁹ bacteriophage treatments, doses were administered on day 0, day 4, and day 7. For the 10¹¹ bacteriophage particles treatment, doses were administered on days 0, 4, and 11 instead of day 7 due to the presence of necrosis on injection site. Tumor sizes were then monitored twice a week using a precise caliper until tumors exceed 1500 mm³ or until day 24 after the first injection. High doses of control synthetic bacteriophages exhibited strong anti-tumoral activities (FIG. 13A-B).

Synthetic bacteriophages displaying anti-checkpoint nanobodies exhibit anti-tumoral activity. To assess the effect of adding a checkpoint inhibitor on the anti-tumoral activity of synthetic bacteriophages, synthetic bacteriophages displaying CD47, PD-L1, and CTLA-4 checkpoint inhibitors nanobodies were developed using the process described in Example 1. Mice were injected subcutaneously with 5×10⁶ A20 cells in their right flanks and were observed every two days to monitor tumor growth. Mice were next divided into two treatment groups, all treatments were administered intratumorally on days 0, 4, and 7. The first group received an effective dose of 10⁹ synthetic bacteriophages displaying the anti-CD47 nanobody on pIII (FIG. 13C). The second group received an effective dose 10¹ of either synthetic bacteriophages displaying the anti-PD-L1 nanobody on pIII or synthetic bacteriophages displaying the anti-CTLA-4 nanobody on pIII (FIG. 13C). Tumor sizes were then monitored twice a week using a precise caliper until tumors are either eliminated or are too large to pursue the experiment. As compared with a control group treated with PBS, or control bacteriophage (10⁹ bacteriophage particles dose as control for CD47, and 10⁸ bacteriophage particles dose a control for PD-L1 and CTLA-4), only the bacteriophages displaying the anti-CD47 nanobody, the anti-PDL1 nanobody, or the anti-CTLA-4 nanobody produced anti-tumoral activities resulting in tumor clearance when compared to appropriate controls. This experiment shows that the presence of checkpoint inhibitors on synthetic bacteriophages potentiate their antitumoral effects.

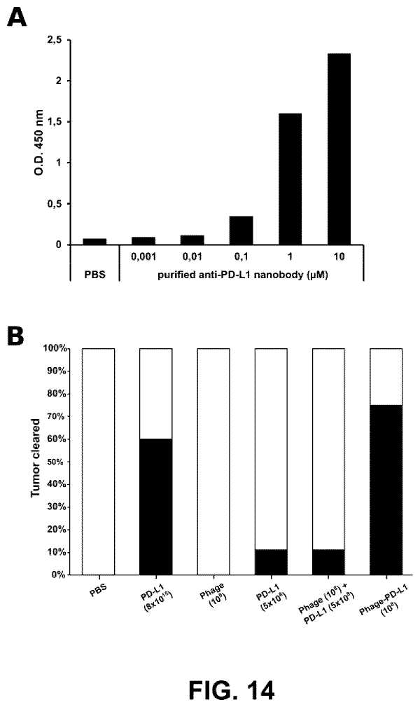

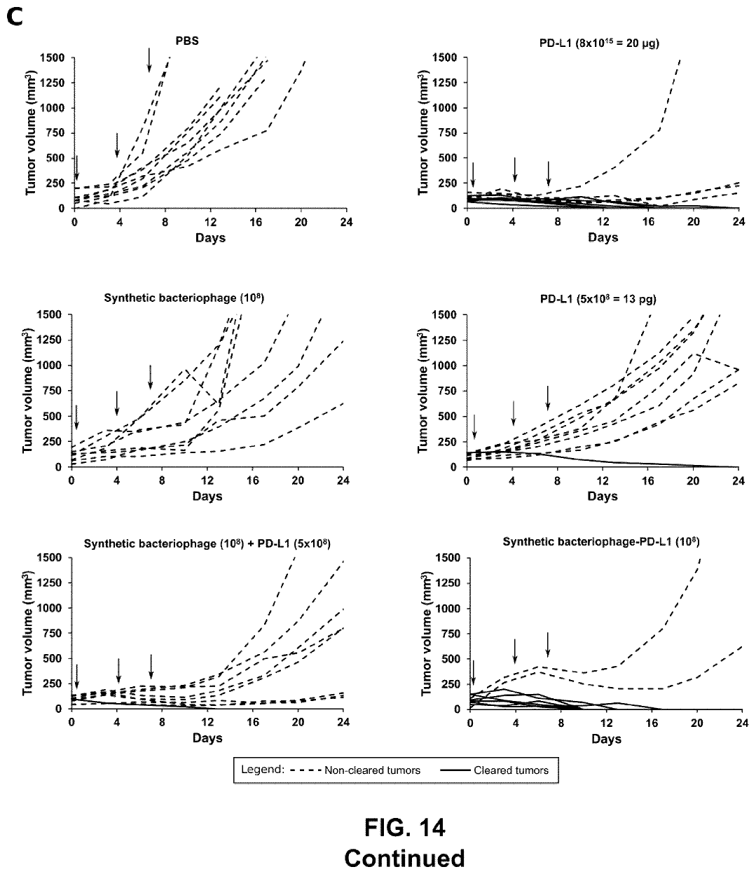

A synergic therapeutic effect is triggered when a checkpoint inhibitor is displayed by a synthetic bacteriophage. As demonstrated in the previous section, synthetic bacteriophages displaying checkpoint inhibitor exhibit improved anti-tumoral efficacy, lowering by a 100 fold factor (10¹⁰ for phage alone vs. 10⁸ for anti-PD-L1 synthetic bacteriophage) the dose needed to clear tumors. We next investigated whether a checkpoint inhibitor displayed by a bacteriophage exhibits the same enhanced therapeutic activity compared to the checkpoint inhibitor administered alone or as a combination therapy. An enhanced activity would suggests a synergistic effect between the checkpoint inhibitor and the bacteriophage. To test this hypothesis, we measured the antitumoral activity of the purified anti-PD-L1 nanobody alone or in conjunction with a bacteriophage. As a control, we first verified that the purified anti-PD-L1 nanobody was functional and confirmed its binding activity on A20 cells by ELISA (FIG. 14A). With the activity of the purified nanobody validated, we next investigated if a synergistic effect was observed when the anti-PD-L1 nanobody is displayed by a bacteriophage (FIG. 14B-C). Mice were injected subcutaneously with 5×10⁶ A20 cells in their right flanks and were observed daily to monitor tumor growth. Mice were next divided into six treatment groups, all treatments were administered intratumorally on days 0, 4, and 7. The first group received 50 μL of PBS alone (control vehicle), the second group received 50 μL of PBS comprising of 8×10¹⁵ anti-PD-L1 nanobody molecules (20 μg, which correspond to a typical treatment dose), the third group received 50 μL of PBS comprising 10⁸ particles of purified control bacteriophage (pTAT002) (mimicks a treatment with 10⁸ particles of anti-PD-L1 synthetic bacteriophage, but without the checkpoint inhibitor), the fourth group received 50 μL of PBS comprising 5×10⁸ of purified anti-PD-L1 nanobody (12.9 pg, mimicks a treatment with 10⁸ particles of anti-PD-L1 synthetic bacteriophage, where 5 anti-PD-L1 nanobody are displayed per bacteriophage, but without the bacteriophage), the fifth group received 50 μL of PBS comprising 5×10⁸ of purified anti-PD-L1 nanobody in conjunction with 10⁸ particles of control bacteriophage (pTAT002) (mimicks a treatment with 10⁸ particles of anti-PD-L1 synthetic bacteriophage with the anti-PD-L1 nanobody, but not displayed by the bacteriophage), and the last group received 50 μL of PBS comprising 10¹ particles of synthetic bacteriophage displaying the anti-PD-L1 nanobody on pIII (pTAT020) (treatment where the checkpoint inhibitor is displayed by the synthetic bacteriophage). As expected, the group of mice treated with PBS did not exhibit any signs of anti-tumoral activity and quickly reached experiment limits. The group of the group of mice that received the very dose of 8×10¹⁵ molecules of purified anti-PD-L1 nanobody alone showed a strong antitumoral effect but no tumor clearance. This experimental data point proved that the purified PD-L1 nanobody was functional. The mice treated with 5×10⁸ molecules of purified anti-PD-L1 nanobody, as well as the group treated with 10⁸ particles of control bacteriophages, showed moderate antitumoral effects and no tumor clearance. The group of mice treated 5×10⁸ molecules of purified anti-PD-L1 nanobody in conjunction with 10⁸ particles of control bacteriophages showed an improved antitumoral effect, which shows that adding bacteriophage to a checkpoint inhibitor treatment potentiate the effects, however no clearance was observed. The group of mice treated with the synthetic bacteriophage displaying the anti-PD-L1 nanobody exhibited the strong antituimoral activity with four tumors cleared in less than 10 days. A treatment dose of 10⁸ particles of synthetic bacteriophage displaying the anti-PD-L1 nanobody exhibits more antitumoral activity than 8×110¹⁵ molecules of anti-PD-L1 nanobody alone, which corresponds to a 8×10⁷ fold dose efficacy improvement. These results, in an unpredictable way, shows that the synthetic bacteriophage potentiate the effect of the checkpoint inhibitor molecule, whether the checkpoint inhibitor is directly displayed by the bacteriophage or not. Also, having the checkpoint inhibitor directly displayed by the bacteriophage further enhance the antitumoral effect in an unpredictable way compared to a treatment where the checkpoint inhibitor in administered in conjunction with the bacteriophage.

### Example 6—Live Biotherapeutic Secretes the Synthetic Bacteriophage Intra-Tumorally and has a Direct Anti-Tumoral Effect

All strains and plasmids used in this Example are described in Table 1. Cells were typically grown in Luria broth Miller (LB) or on Luria broth agar Miller medium supplemented, when needed, with antibiotics at the following concentrations: ampicillin (Ap) 100 μg/mL, chloramphenicol (Cm) 34 μg/mL, kanamycin (Km) 50 μg/mL, nalidixic acid (Nx) 4 μg/mL, spectinomycin (Sp) 100 μg/mL, streptomycin (Sm) 50 μg/mL, sulfamethoxazole (Su) 160 μg/mL, tetracycline (Tc) 15 μg/mL, and trimethoprim (Tm) 32 μg/mL. All cultures were routinely grown at 37° C. for no longer than 18 hours before use in the experiments. Bacteriophages were extracted from confluent bacterial culture (grown overnight) using the PEG precipitation protocol as detailed in example II. A20 lymphocyte B lymphoma cells were ordered from ATCC (TIB-208). Cells were maintained at density between 2×10³ cell/mL and 2×10⁶ cell/mL throughout all the experiments in RPMI-1640 supplemented with 10% Fetal Bovine Serum (FBS) and 0.05 mM 2-mercaptoethanol.

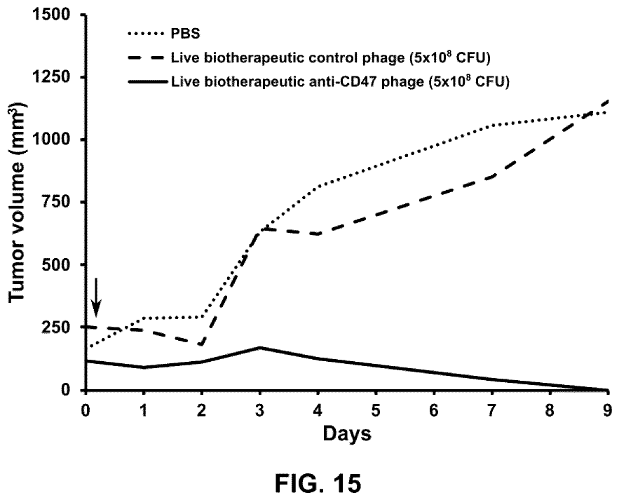

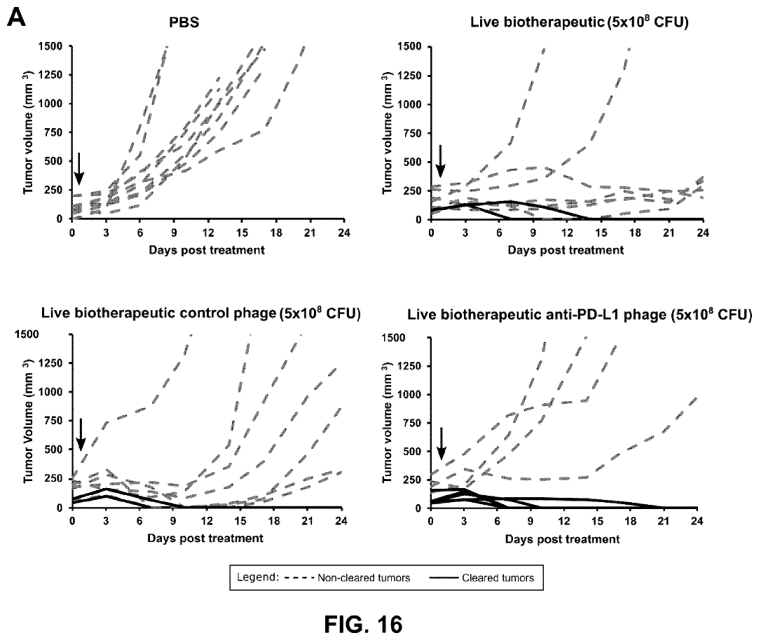

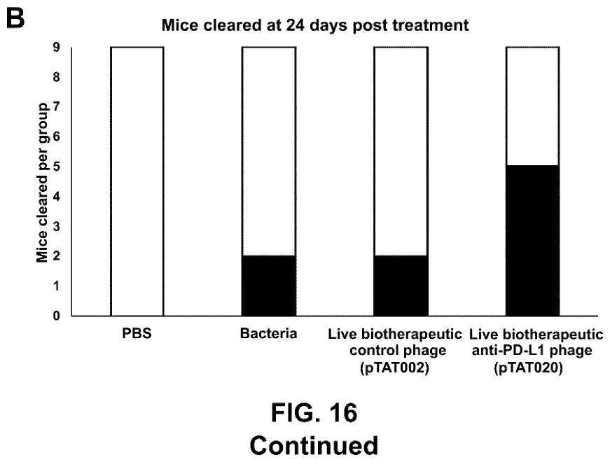

The live biotherapeutic secreting synthetic bacteriophages displaying checkpoint inhibitors can reduce the size of solid tumors. To measure the anti-tumoral effect of live biotherapeutics secreting synthetic bacteriophages displaying checkpoint inhibitors developed using the process described herein, mice were injected subcutaneously with 5×10⁶ A20 cells in their flanks and were observed daily to monitor tumor growth. The mice were next divided into three treatment groups, all treatments were administered a single dose intratumorally on day 0 of the experiment when tumors reached 75-200 mm³. The first group received only PBS in the tumor (vehicle control), the second group received 5×10⁸ CFU of live biotherapeutics secreting a control bacteriophage that did not display any checkpoint inhibitor, and the last group received 5×10⁸ CFU of live biotherapeutics secreting a synthetic bacteriophage displaying one or more immune checkpoint inhibitors. Tumor sizes were then monitored twice a week using a precise caliper until the tumor reached 1500 mm³, or until day 24 post treatment. The experiment was performed using different versions of the synthetic bacteriophage. The first version of the system uses a synthetic bacteriophage displaying an anti-CD47 nanobody on the pIII coating protein (as described in previous examples with pTAT003). Soon after the injection of a single dose of the live biotherapeutic secreting the anti-CD47 synthetic bacteriophage, the tumor volume started to shrink. This live biotherapeutic was able of specifically eliminating the tumors within 9 days post-treatment whereas tumors treated with either PBS or the live biotherapeutic secreting control bacteriophages were not eliminated (FIG. 15). The same experiment was performed using a live biotherapeutics secreting synthetic bacteriophages displaying an anti-PD-L1 nanobody on the pIII coating protein. This time, mice bearing A20 tumors were treated with either PBS, 5×10⁸ CFU of unmodified bacteria, 5×10⁸ CFU of bacteria secreting a control bacteriophage (pTAT002), or 5×10⁸ CFU of bacteria secreting a bacteriophage displaying the anti-PD-L1 nanobody (pTAT020) (FIG. 16A-B). The treatment with the live biotherapeutics secreting the synthetic bacteriophages displaying an anti-PD-L1 nanobody was able to clear 5 of 9 mice, proving its efficacy. These data demonstrate that synthetic therapeutic bacteriophages can be delivered locally using a live biotherapeutic approach.

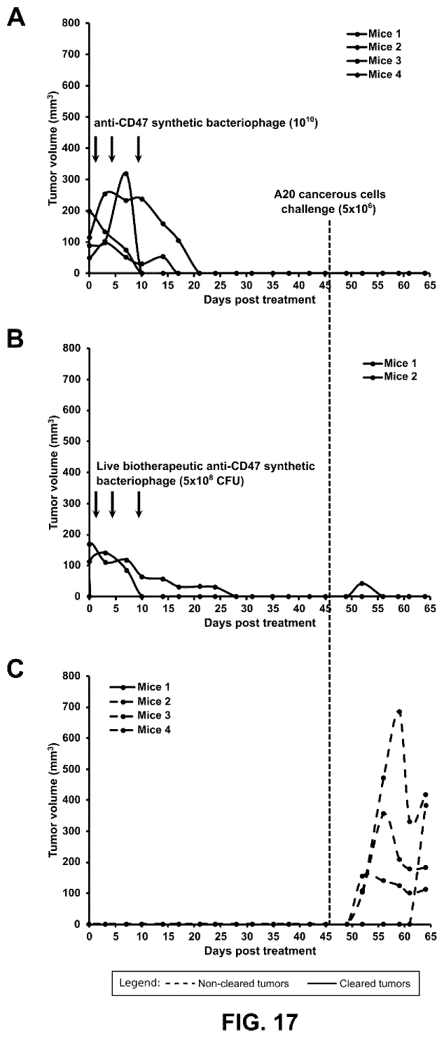

The synthetic therapeutic bacteriophage can elicit a complete adaptative immune response against cancer cells. To test if treatments with the synthetic bacteriophage displaying an anti-CD47 nanobody, or the live biotherapeutic secreting the synthetic therapeutic bacteriophage displaying an anti-CD47 nanobody, can trigger an adaptive immune response against A20 cancerous cells, mice cleared by these intra-tumoral treatments were rechallenged at day 46 post treatment with an injection of 5×10⁶ A20 cells in their left flank (FIGS. 17A and B). As control, naïve mice were also challenged by injecting 5×10⁶ A20 cells in their right flank (FIG. 17C). Tumor growth was monitored twice a week in both groups to detect the formation of any tumor. Both treatments, either intra-tumoral injection of the synthetic therapeutic bacteriophage, or intra-tumoral injection of the live biotherapeutic secreting the synthetic bacteriophages, elicited a complete adaptive response preventing the formation of new tumors (FIG. 17).

### Example 7—Live Biotherapeutic Secrete a Synthetic Bactriophage Displaying a Therapeutic Enzyme with Anti-Tumoral Activity

Bacterial cells were typically grown in Luria broth Miller (LB) or on Luria broth agar Miller (LBA) medium supplemented, when needed, with antibiotics at the following concentrations: ampicillin (Ap) 100 μg/mL, chloramphenicol (Cm) 34 μg/mL, kanamycin (Km) 50 μg/mL, nalidixic acid (Nx) 4 μg/mL, spectinomycin (Sp) 100 μg/mL, streptomycin (Sm) 50 μg/mL, sulfamethoxazole (Su) 160 μg/mL, tetracycline (Tc) 15 μg/mL, and trimethoprim (Tm) 32 μg/mL. All cultures were routinely grown at 37° C. for no longer than 18 hours before use in the experiments. Bacteriophages were extracted from confluent bacterial culture (grown overnight) using the PEG precipitation protocol described in Example 2. The bacteriophage preparation was used immediately after precipitation. A20 lymphocyte B lymphoma cells were ordered from ATCC (TIB-208). Cells were maintained at density between 2×10⁵ cell/mL and 2×10⁶ cell/mL throughout all the experiments in RPMI-1640 supplemented with 10% Fetal Bovine Serum (FBS) and 0.05 mM 2-mercaptoethanol. Cells were typically grown in Luria broth Miller (LB) supplemented, when needed, with antibiotics at the following concentrations: kanamycin (Km) 50 μg/mL, spectinomycin (Sp) 100 μg/mL All cultures were routinely grown at 37° C. with agitation (200 rpm). No bacterial cultures over 18 hours of age were used in the experiments.

Ni-nitrilotriacetic acid (NI-NTA) beads purification of synthetic bacteriophage particles. For the purification of synthetic bacteriophage using Ni-NTA beads, overnight cultures (20 mL) of live biotherapeutics secreting the synthetic bacteriophage were transferred in 50 mL tubes and then centrifuged at 6000 rpm for 10 min. Subsequently, 18 mL of supernatants were transferred into new 50 mL tubes and 2 mL of PBS 10× was added to buffer the pH. Then, 0.250 mL of Ni-NTA resin (50% slurry in PBS) were added to each tubes and all samples were incubated 2 h30 at 4° C. with agitation. The tubes were then centrifuged at 4000 rpm for 2 min and the supernatants were removed. The beads were resuspended in 1 mL of PBS, transferred in 1.5 mL tubes, centrifuged at 4000 rpm for 2 min. The supernatants were discarded and the beads were resuspended in 1 mL of PBS. The beads with the synthetic bacteriophages bound onto them are ready for subsequent assays.

5-fluorocytosine (S-FC) to 5-fluorouracil (5-FU) conversion assay. To measure the conversion of 5-FC into 5-FU by the synthetic bacteriophage, 250 μL of Ni-NTA beads bound with synthetic bacteriphages were added to 1.5 mL test tubes, as well as 300 μL of 5-fluorocytosine (5-FC) 6 mM (12% DMSO). The tubes were then mixed, quick spinned, and 50 μL of supernatant was transferred to spectrophotometer cuvettes pre-filled with 1 mL of HCl 0,1 N (this is used to measure 5-FC/5-FU ratio at T₀). The samples were then resuspended by doing up and downs and incubated 24 h at 37° C. In parallel, a blank comprising 1 mL of HCl 0,1 N, and 50 μL of PBS/DMSO (12%), was prepared and the OD of the blank, and the collected samples, were read at 255 nm and 290 nm to determine the concentrations of 5-FC and 5-FU at T₀. The next day, after 24 h of incubation, the samples were quick spinned and 50 μL of supernatant were transferred to spectrophotometer quartz cuvettes pre-filled with 1 mL of HCl 0,1 N. Then the OD of the samples was measured at 255 nm and 290 nm against the blank solution composed of 1 mL of HCl 0,1 N and 50 μL of PBS/DMSO (12%). The percentage of 5-FC and 5-FU were then calculated using the formulas %5-FC=[5-FC]_{24 h}/[5-FC]_{0 h}=(0.119×A290−0.025×A255)_{24 h}/(0.119×A290−0.025×A255)_{0 h} and %5-FU=[5-FU]_{24 h}/[5-FU]_{0 h}=(0.185×A255−0.049×A290)_{24 h}/(0.185×A255−0.049×A290)_{0 h}.

5-FU antiproliferative assay. To test the antiproliferative effect of the 5-FU obtained after conversion of 5-FC by the cytosine deaminase (codA), a 96 wells plate was seeded with 104 A20 cells per well and treated in triplicates with either the 5-FU conversion product at a final concentration of 200 M, 5-FC at 200 μM (control), or an equivalent volume of PBS 12% DMSO (control). The plate was then incubated at 37° C. with 5% CO2 for 42 h. After that incubation period, cell viability was measured using trypan blue. Briefly, 100 μL of cell suspension was collected and mixed with 100 μL of trypan blue 0.4%. Viable cells were then counted using an hemacytometer.

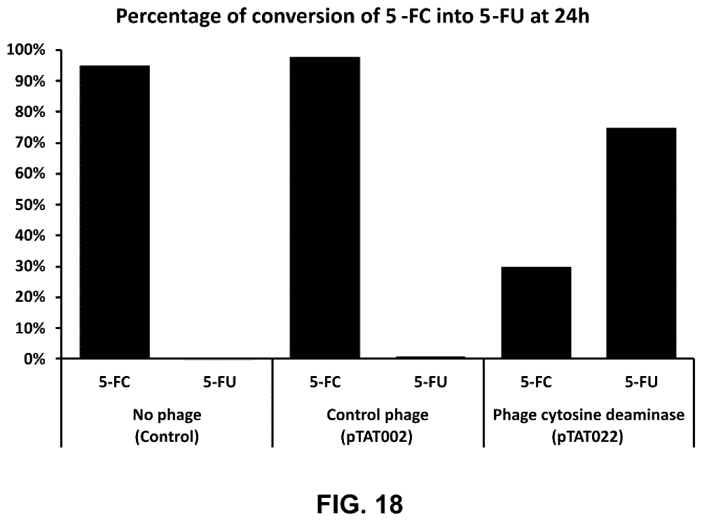

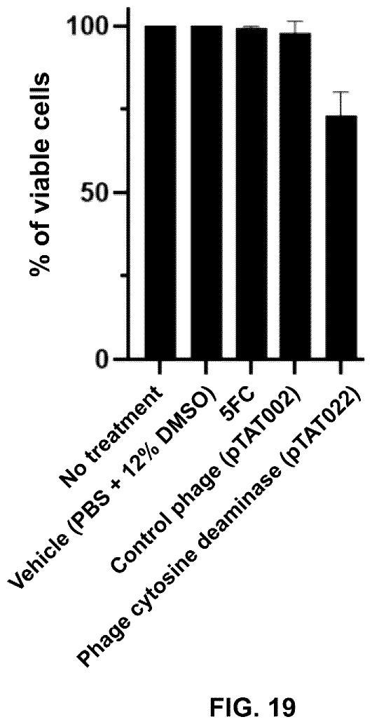

The live biotherapeutics secreting a synthetic therapeutic bacteriophage displaying the cytosine deaminase converts the 5-FC precursor into the chemotherapeutic agent 5-FU. Synthetic bacteriophages derived from pTAT002, acting as control, or from pTAT022, displaying of the cytosine deaminase on pIII (codA), were purified using Ni-NTA beads and incubated with 5-FC 6 mM (12% DMSO). After 24 h of incubation, the percentages of 5-FC and 5-FU were determined by measuring the OD at 255 nm and 290 nm. Only the synthetic bacteriophage displaying the cytosine deaminase was able to convert the 5-FC precursor into the chemotherapeutic agent 5-FU (FIG. 18), proving that the synthetic bacteriophage produced by the live biotherapeutic can by used to deliver therapeutic enzymes. The S-FU produced by the synthetic therapeutic bacteriophage displaying the cytosine deaminase is active and has an anti-tumoral activity. The antitumoral activity of the 5-FU produced by the synthetic bacteriophage displaying the cytosine deaminase was tested on cancer cells. A20 cancer cells were incubated for 42 h with 200 μM of 5-FU converted by the synthetic bacteriophage displaying the cytosine deaminase, or with an equivalent volume of reaction mix obtained with the control synthetic bacteriophage, or with equivalent volume of vehicle (PBS 12% DMSO), or with 200 μM of 5-FC. Cancer cell death was then monitored by trypan blue (FIG. 19). Cancer cell death was only observed with the synthetic bacteriophage displaying the cytosine deaminase, proving that the enzyme converted the 5-FC precursor into active 5-FU. The synthetic bacteriophage can thus be used to deliver enzymes with anti-cancer activities.

### Example 8—Secretion of Synthetic Therapeutic Bacteriophage by the Live Biotherapeutic can be Improved by Using Alternative Start Codon

Bacterial cells were typically grown in Luria broth Miller (LB) or on Luria broth agar Miller (LBA) medium supplemented, when needed, with antibiotics at the following concentrations: ampicillin (Ap) 100 μg/mL, chloramphenicol (Cm) 34 μg/mL, kanamycin (Km) 50 μg/mL, nalidixic acid (Nx) 4 μg/mL, spectinomycin (Sp) 100 μg/mL, streptomycin (Sm) 50 μg/mL, sulfamethoxazole (Su) 160 μg/mL, tetracycline (Tc) 15 μg/mL, and trimethoprim (Tm) 32 μg/mL. All cultures were routinely grown at 37° C. for no longer than 18 hours before use in the experiments. Bacteriophages were extracted from confluent bacterial culture (grown overnight) using the PEG precipitation protocol described in Example 2. The bacteriophage preparation was used immediately after precipitation.

Synthetic bacteriophage titration by Enzyme Linked Immunosorbent Assay (ELISA). Detection and quantification of bacteriophage expression was performed using the commercially available Phage Titration ELISA kit (PRPHAGE, Progen) following manufacturer's instructions. Briefly, lyophilized M13 particles were resuspended at 1.5×10⁸ phage/mL as per manufacturer's recommendation and diluted 1/2 serially to generate a standard curve. Bacteriophage preparations were next diluted 1/10, 1/100, 1/1000, and 1/10000 and added (as well as the standard curve) to mouse anti-M13 pre-coated ELISA wells. Bacteriophage particles captured were detected by a peroxidase conjugated monoclonal anti-M13. After addition of tetramethylbenzidine the optical density of each well was measured at 450 nm using the Biotek plate reader instrument. The procedure was repeated using an antibody HA-Tag (6E2) Mouse mAb (HRP Conjugate) (1: 1000 Cell Signaling Technology, Danvers, MA, USA) instead of the anti-M13-HRP provided with the kit to detect the modified pIII protein at the surface of engineered phages.

Verification of fusion protein integrity by western blot. Bacteria were grown overnight at 37° C. with agitation in LB broth supplemented with kanamycin and spectinomycin. The bacteria were pelleted by centrifugation and the culture supernatants were transferred in a new tube and buffered with concentrated PBS. The phages displaying an hexahistidine tag were pulled-down from the supernatants using Ni-NTA beads by incubating 2 hours at 4° C. with agitation. The beads were then washed 3 times with PBS and the phages were eluted by denaturation using sample buffer 4X (SB4X). The samples were denatured for 1 hour at 65° C. and loaded on a 15% acrylamide gel. The gel migration of the samples was performed for 1 hour at 150 volts. The proteins were then transferred on a 0.2 μm nitrocellulose membrane by applying 100 volts for 1 hour. The membrane was air dried to let evaporate any trace of methanol and block for 1 hour in TBS—0.1% Tween 20 —4% dried milk at 4° C. with agitation. The membrane was transferred in a western blot sealable bag and incubated over night with anti-HA-HRP (Cell Signaling) diluted in blocking buffer at 4° C. with agitation. After three TBS—0.1% Tween 20 washes, the membrane revelation was performed by applying the Immobilon ECL Ultra Western HRP substrate and the image acquired with the Vilber Fusion FX apparatus. The images were processed using the Image Lab software.

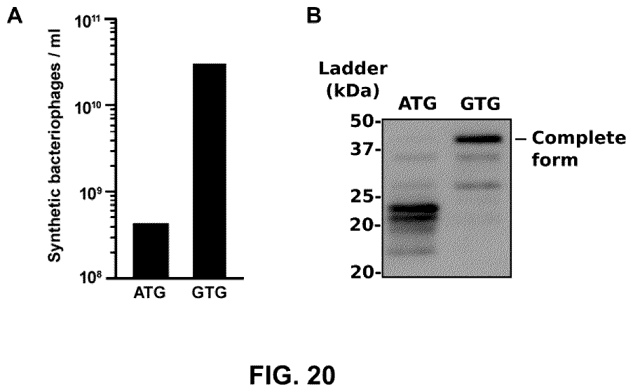

Synthetic bacteriophage secretion is improved by having a GTG start codon instead of an ATG start codon for the displayed protein. ATG is the normal start codon for a protein, leading to the highest traduction level. In some instances, expressing too much of a therapeutic protein fused to the bacteriophage coating protein can be toxic and have a detrimental effect on the bacterial host, which in the end results in poor bacteriophage secretion. GTG is an alternative start codon which leads to lower level of protein traduction (Hecht et al. Nucleic Acids Research, 2017, Vol. 45, No. 7 3615-3626; incorporated herein by reference). Furthermore, GTG as a start codon relies on start tRNA wobble to allow traduction initiation from the wrong codon. This stalls ribosomes and might allow for improved ribosome trafficking on the gene, thus producing more complete protein products. To test the effect of the start codon on the secretion of the therapeutic bacteriophage, overnight production of synthetic bacteriophages displaying the anti-PD-L1 nanobody fused to pIII with an ATG start codon (pTAT020), or with a GTG start codon (pTAT020-GTG) where PEG precipitated and then quantified by ELISA. Results show that the presence of the GTG codon increases by a factor 100 the secretion of the therapeutic bacteriophage (FIG. 20A). The integrity of the anti-PD-L1 nanobody displayed on the surface of bacteriophages on pIII was next investigated by western blot. The bacteriophage derived from pTAT020 and from pTAT020-GTG were both purified on Ni-NTA beads and released by heat denaturation in SB4X. Then, samples were analysed on SDS-PAGE and western blot using an anti-HA antibody to investigate protein integrity (FIG. 20B). Several bands revealed for the pTAT020 construct with the one corresponding to the complete protein product being present at lower concentration. On the other hand, the highest band (corresponding to the complete protein product) represented the majority of the protein product for pTAT020-GTG, supporting that using alternative start codon improve ribosome trafficking, might lower proteolysis and maximize secretion of the therapeutic bacteriophages.

### Example 9—Synthetic Bacteriophage Secretion System can Produce Bacteriophage Displaying Therapeutic Proteins or Two or More Minor Coat Proteins

All strains and plasmids used in this Example are described in Table 1. Cells were typically grown in Luria broth Miller (LB) or on Luria broth agar Miller medium supplemented, when needed, with antibiotics at the following concentrations: ampicillin (Ap) 100 μg/mL, chloramphenicol (Cm) 34 μg/mL, kanamycin (Km) 50 μg/mL, nalidixic acid (Nx) 4 μg/mL, spectinomycin (Sp) 100 μg/mL, streptomycin (Sm) 50 μg/mL, sulfamethoxazole (Su) 160 μg/mL, tetracycline (Tc) 15 μg/mL, and trimethoprim (Tm) 32 μg/mL. All cultures were routinely grown at 37° C. for no longer than 18 hours before use in the experiments. Bacteriophages were extracted from confluent bacterial culture (grown overnight) using the PEG precipitation protocol presented in example 2. A20 lymphocyte B lymphoma cells were ordered from ATCC (TIB-208). Cells were maintained at densities between 2×10⁵ cell/mL and 2×10⁶ cell/mL throughout all the experiments in RPMI-1640 supplemented with 10% Fetal Bovine Serum (FBS) and 0.05 mM 2-mercaptoethanol.

Synthetic bacteriophage titration by Enzyme Linked Immunosorbent Assay (ELISA). Detection and quantification of bacteriophage expression was performed using the commercially available Phage Titration ELISA kit (PRPHAGE, Progen) following manufacturer's instructions. Briefly, lyophilized M13 particles were resuspended at 1,5×10⁸ phage/mL as per manufacturer's recommendation and diluted 1/2 serially to generate a standard curve. Bacteriophage preparations were next diluted 1/10 and 1/100, and then added (as well as the standard curve) to mouse anti-M13 pre-coated ELISA wells. Bacteriophage particles captured were detected by a peroxidase conjugated monoclonal anti-M13. After addition of tetramethylbenzidine, the optical density of each well was measured at 450 nm using the Biotek plate reader instrument. The procedure was repeated using an antibody HA-Tag (6E2) Mouse mAb (HRP Conjugate) (1:1000 Cell Signaling Technology, Danvers, MA, USA) instead of the anti-M13-HRP provided with the kit to detect the modified pIII protein at the surface of engineered phages.

Assessment of synthetic bacteriophage binding to PDL1 by ELISA. To measure the binding activity of a synthetic bacteriophage directed against the checkpoint PDL1, an ELISA assay was devised. A 96 well plate was first coated with 1×10⁵ A20 cells resuspended in ice cold PBS for 30 minutes at room temperature. The supernatant was next gently removed by aspiration and cells were fixed to the plate using 10% neutral buffered formalin for 10 minutes at room temperature. The plate was then washed 1 time with 200 μL of PBS. To prevent unspecific binding, the plate was subsequently incubated with 200 μL of blocking buffer (PBS, 3% skimmed milk, 1% BSA) 1 hour at RT. Blocking was stopped by removing the blocking buffer and washing the plate two times with 200 μL of TBS-T. Then, 100 μL of PEG precipitated synthetic bacteriophages diluted in the PBS 1× and displaying an anti-CTLA-4 anticalin on pIX and a nanobody against PDL1 on pIII (pTAT032+pTAT035) were added to wells comprising the A20 cells, and incubated for 1 h at RT. A control without displayed nanobodies against PDL1 was also performed (pTAT004+pTAT002). The plate was then washed 3 times with 200 μL of PBS and 100 μL of Anti-FLAG-HRP (M2, Sigma-aldrich) diluted in blocking buffer (1:500) were added. The anti-FLAG-HRP (M2, Sigma-aldrich) was preferred here to measure only the complete bacteriophage by quantifying the presence of pIX-FLAG-anticalin CTLA-4, hereby confirming that both the nanobody and the anticalin are present on the same bacteriophage particles. The plate was incubated 1 h at RT in the dark and then washed 5 times with TBS-T. To measure the presence of the synthetic bacteriophages, 100 μL of TMB substrate solution (ThermoFisher) was added to each well and the plate was incubated 15 min at RT. The reaction was stopped by adding 100 μL of stop solution (0.5 M H₂SO₄) to each well. The absorbance was then measured at 450 nm.

Assessment of synthetic bacteriophage binding to CTLA-4 by ELISA. To measure the binding activity of a synthetic bacteriophage directed against the checkpoint CTLA-4, an ELISA assay was devised. A 96 well plate was first coated overnight at 4° C. with recombinant CTLA-4 protein (R&D systems) diluted at a 10 μg/mL in coating buffer (0.05 M Carbonate-Bicarbonate at pH 9.6). The plate was then washed 3 times with 200 μL of TBS-T. To prevent unspecific binding, the plate was subsequently incubated with 200 μL of blocking buffer (TBS-T, 3% skimmed milk, 1% BSA) 1 hour at RT. Blocking was stopped by removing the blocking buffer and washing the plate two times with 200 μL of TBS-T. Then, 100 μL of PEG precipitated synthetic bacteriophages diluted in the TBS 1× and displaying an anti-CTLA-4 anticalin on pIX and a nanobody against PDL1 on pIII (pTAT032+pTAT035) were added to wells comprising the CTLA-4 protein, and incubated for 1 h at RT. A control without displayed anticalin against CTLA-4 was also performed (pTAT004+pTAT002). The plate was then washed 3 times with 200 μL of TBS-T and 100 μL of Anti-HA-HRP (Cell Signaling) diluted in blocking buffer (1:500) were added. The anti-HA-HRP (Cell Signaling) was preferred here to measure only the complete bacteriophage by quantifying the presence of pIII-HA-nbPDL1, hereby confirming that both the nanobody and the anticalin are present on the same bacteriophage particles. The plate was incubated 1 h at RT in the dark and then washed 5 times with TBS-T. To measure the presence of the synthetic bacteriophages, 100 μL of TMB substrate solution (ThermoFisher) was added to each well and the plate was incubated 15 min at RT. The reaction was stopped by adding 100 μL of stop solution (0.5 M H₂SO₄) to each well. The absorbance was then measured at 450 nm.

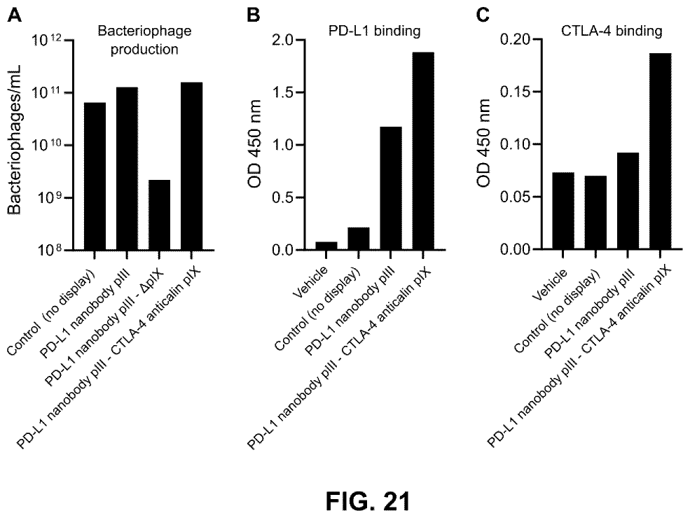

The live biotherapeutic secretes bi-specific synthetic bacteriophages, which displays an anti-PDL1 nanobody on pIII and an anti-CTA-4 on pIX that bind simultaneously to the immune checkpoint PDL1 and CTL-4. The previous examples showed that the synthetic bacteriophage secretion system can produce bacteriophage particles that bind to several molecular targets through different binding proteins. The next step was thus to show that those displays could be combined on the same bacteriophage, which could then bind to two or more immune checkpoints. An examplified version of a double display system requires a bacteriophage secretion machinery lacking both wildtype pIII (located on the tail of the bacteriophage) and pIX (located at the head of the bacteriophage) subunits to maximise display efficiency. As such, pTAT032AgpIX, a plasmid lacking gpIX and displaying a nanobody against PDL1 on pIII was used as the bacteriophage secretion machinery. The interruption of phage secretion in pTAT032AgpIX by the absence of gpIX gene was first assess by anti-pVIII ELISA assay. Plasmid pTAT032AgpIX produced low titers of bacteriophages as compared to it parental construct pTAT032, supporting the gpIX was successfully impaired (FIG. 21A). By combining pTAT032ΔgpIX (bacteriophage machinery deficient for pIX and displaying the anti-PDL1 nanobody on pIII) with pTAT035 (anti-CTLA-4 anticalin displayed on pIX), we next sought to test if the bacteriophage particle can display multiple functional recombinant proteins. Those plasmids were combined in MG1655 and bacteriophage particles were produced and purified by PEG precipitation. Next, a set of three ELISA were performed using bacteriophage displaying no protein as a control. The first ELISA used an anti-pVIII B62-FE3 (progen) antibody immobilized in the wells of the plate and an anti-PVIII-HRP B62-FE3 (progen) antibody for revelation to dose phage production (FIG. 21A). A second ELISA was performed with A20 cells attached to the wells of the plates. A20 cells express PD-L1, on which bacteriophage particles derived from the double display system bind. Then bacteriophages are revealed using an anti-pVIII-HRP B62-FE3 (progen) antibody, which bind to the pVIII to reveal bacteriophage particles attached to the A20 cells (FIG. 21B). The last ELISA is performed with a recombinant CTLA-4 purified protein attached to the wells of the plate. The bacteriophages expressing a CTLA-4 binding protein binds to the purified protein and are then revealed with an anti-HA-HRP (Cell Signaling) antibody, which binds to the pIII-HA-PD-L1 nanobody, thus revealing only complete bacteriophage particles bound to the CTLA-4 protein and also displaying the PD-L1 nanobody (FIG. 30 21C). Although both the control and the bacteriophage particles derived from the combination of pTAT032ΔgpIX (bacteriophage machinery deficient for pIX and displaying the anti-PDL1 nanobody on pIII) with pTAT035 (anti-CTLA-4 anticalin displayed on pIX) produced bacteriophage particles, only the latter could produce bacteriophages binding to both CTLA-4 and PDL1. Together, these results show that bacteriophages can display several functional therapeutic proteins at the same time on the same bacteriophage particle.

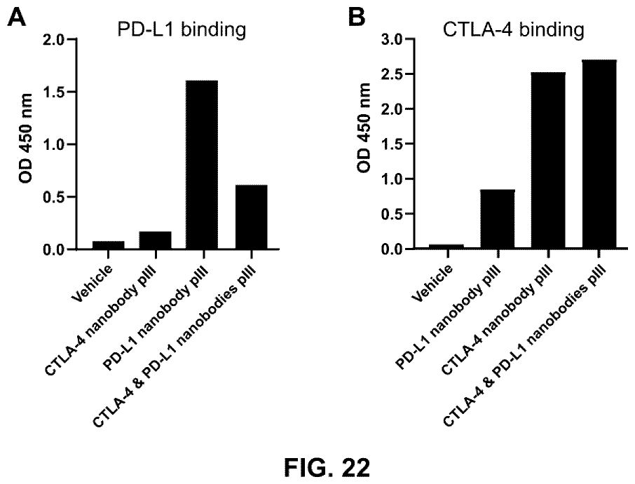

The live biotherapeutic secretes bi-specific synthetic bacteriophages, which display anti-PD-L1 and anti-CTLA-4 nanobodies on pII, that bind simultaneously to the immune checkpoint PD-L1 and CTLA-4. The synthetic therapeutic bacteriophage particles can display a mix of different therapeutic proteins on the same coating protein. To illustrate this, plasmid pTAT032 (displaying the nanobody against PD-L1 on pIII) and the plasmid pTAT019 (displaying the nanobody against CTLA-4 on pIII) were combined in a same bacterium. The resulting cells thus secretes synthetic therapeutic bacteriophage which can display both PD-L1 and CTLA-4 nanobodies on pill. To test this hypothesis, a first ELISA was performed on A20 cells, since A20 cells express PD-L1 on which bacteriophage particles derived from the double display system should bind. Bacteriophages were revealed using an anti-pVIII-HRP B62-FE3 (progen) antibody, which binds to the pVIII coating protein to reveal bacteriophage particles attached to the A20 cells (FIG. 22A). A second ELISA was performed on recombinant CTLA-4 protein. Bacteriophages expressing an anti-CTLA-4 nanobody bound to the purified protein and were then revealed with an anti-pVIII-HRP (Cell Signaling) antibody, which reveals complete bacteriophage particles bound to the CTLA-4 protein (FIG. 22B). As a control, bacteriophages displaying only the nanobody against PD-L1 (pTAT032), or only the nanobody against CTLA-4 (pTAT019), were also evaluated. As expected, these controls only produced strong binding signals when tested against their corresponding target. In contrast, the strain displaying both the anti-PD-L1 and anti-CTLA-4 nanobodies on pIII (pTAT032+pTAT019-GTG) was able to bind PD-L1 and CTLA-4 targets in both ELISA tests, supporting that double specific synthetic bacteriophages can be produced using a mix of pIII coating proteins displaying different nanobodies.

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

1. A synthetic therapeutic bacteriophage displaying at least one therapeutic agent, wherein the at least one therapeutic agent is fused to a coating protein of the synthetic bacteriophage.

2. The synthetic therapeutic bacteriophage of claim 1, wherein the synthetic therapeutic bacteriophage is a filamentous bacteriophage.

3. The synthetic therapeutic bacteriophage of claim 1, wherein the synthetic therapeutic bacteriophage comprises a bacteriophage secretion system comprising a bacteriophage machinery.

4. The synthetic therapeutic bacteriophage of claim 3, wherein the bacteriophage machinery comprises a bacteriophage assembly module, a bacteriophage replication module, a bacteriophage coating module, and a therapeutic module.

5. The synthetic therapeutic bacteriophage of claim 4, wherein the therapeutic module comprises the at least one therapeutic agent to be displayed by the synthetic therapeutic bacteriophage.

6. The synthetic therapeutic bacteriophage of claim 5, wherein the therapeutic module comprises one or more bacteriophage coating genes selected from gpIII, gpVI, gpVII, gpVIII, and gpIX, or a portion thereof, respectively coding for coating protein pIII, pVI, pVII, pVIII, and pIX or coding for a portion thereof.

7.-9. (canceled)

10. The synthetic therapeutic bacteriophage of claim 3, wherein the bacteriophage machinery further comprises a regulatory module having regulatory elements controlling activity of the bacteriophage machinery.

11. (canceled)

12. The synthetic therapeutic bacteriophage of claim 1, wherein the therapeutic agent is a binding protein, an antibody, an antibody mimetic, a nanobody, an anticalin, a peptide, or an enzyme which produce an antitumoral activity.

13. The synthetic therapeutic bacteriophage of claim 12, wherein the binding protein binds to one or more proteins, peptides, or molecule involved in carcinogenesis, development of cancer, or of metastases.

14. The synthetic therapeutic bacteriophage of claim 13, wherein the one or more proteins, peptides, or molecule inhibits one or more molecules selected from: CSF1, CSF1R, CCR4, CCL2, CCL17, CCL22, HER2, GD2, IL-10, IL-6, IL-10, IL-13, IL-17, IL-27, IL-35, CD20, CD27, CD30, CD33, CD70, TGF-β, M-CSF, EGFR, ERBB2, ERBB3, PGE2, VEGF, VEGFR-2, CXCR4/CXCL12, Tie2, galectin-1, galectin-3, Phosphatidyl serine, and TAM and Tim Phosphatidyl serine receptors.

15. The synthetic therapeutic bacteriophage of claim 12, wherein the binding protein acts as agonists to activate co-stimulatory receptor that lead to the elimination of cancerous cells.

16. The synthetic therapeutic bacteriophage of claim 15, wherein the one or more co-stimulatory cellular receptors are selected from CD40, CD27, CD28, CD70, ICOS, CD357, CD226, CD137, and CD134.

17. The synthetic therapeutic bacteriophage of claim 12, wherein the binding protein inhibits an immune checkpoint molecule.

18. The synthetic therapeutic bacteriophage of claim 17, wherein the immune checkpoint molecule is selected from CCR4, CTLA-4, CD80, CD86, PD-1, PD-L1, PD-L2, TIGIT, VISTA, LAG-3, TIM1, TIM3, CEACAM1, LAIR-1, HVEM, BTLA, CD47, SIRPα, CD160, CD200, CD200R, CD39, CD73, B7-H3, B7-H4, IDO, TDO, KIR, and A2aR.

19. The synthetic therapeutic bacteriophase of claim 18, wherein the immune checkpoint molecule is CD47.

20. The synthetic bacteriophage of claim 18, wherein the immune checkpoint molecule is PD-L1.

21. The synthetic bacteriophage of claim 18, wherein the immune checkpoint molecule is CTLA-4.

22.-26. (canceled)

27. The synthetic therapeutic bacteriophage of claim 12, wherein the peptide is a Tumor Associated Antigen (TAA).

28. (canceled)

29. The synthetic therapeutic bacteriophage of claim 1, wherein the therapeutic agent is the cytosine deaminase.

30. A live biotherapeutic for producing at least one therapeutic agent, the live biotherapeutic comprising a recombinant bacterial organism comprising a bacteriophage secretion system capable of secreting the synthetic therapeutic bacteriophage of claim 1.

31.-47. (canceled)

48. A method for delivering at least one therapeutic agent to a tumor site in a subject, the method comprising administering an effective amount of the synthetic therapeutic bacteriophage of claim 1 or an effective amount of the live biotherapeutic of claim 30 to the subject in need thereof.

49.-54. (canceled)
